(19) United States
(12) Patent Application Publication
(10) Pub. No.: US 2013/0267584 A1
(43) Pub. Date: Oct. 10, 2013

(54) APTAMERS TO TISSUE FACTOR PATHWAY INHIBITOR AND THEIR USE AS BLEEDING DISORDER THERAPEUTICS

(71) Applicants: BAXTER INTERNATIONAL INC., Deerfield, IL (US); BAXTER HEALTHCARE S.A., Glattpark (Opfikon) (CH)

(72) Inventors: Michael Dockal, Vienna (AT); Friedrich Scheiflinger, Vienna (AT)

(73) Assignees: Baxter Healthcare S.A., Glattpark (Opfikon) (CH); Baxter International Inc., Deerfield, IL (US)

(21) Appl. No.: 13/797,414

(22) Filed: Mar. 12, 2013

Related U.S. Application Data
(60) Provisional application No. 61/614,438, filed on Mar. 22, 2012.
(60) Provisional application No. 61/667,823, filed on Jul. 3, 2012.

(51) Int. Cl. C12N 15/115

(52) U.S. Cl. CPC C12N 15/115
USPC 514/44 R; 436/501

(57) ABSTRACT

The present invention provides compositions and methods for modulating TFPI protein plasma concentration and TFPI protein function. Such modulation can be used to treat blood disorders such as bleeding disorders and clotting disorders.

### BACKGROUND OF THE INVENTION

Coagulation is the formation of a stable fibrin/cellular hemostatic plug that is sufficient to stop bleeding. The coagulation process involves complex biochemical and cellular interactions that can be divided into three stages. Stage 1 is the formation of activated Factor X by either the contact (intrinsic) or the tissue factor/VIIa (extrinsic) pathway. Stage 2 is the formation of thrombin from prothrombin by Factor Xa. Stage 3 is the formation of fibrin from fibrinogen stabilized by Factor XIIIa.

Hemophilia is defined as a congenital or acquired disorder of coagulation that usually, but not always, involves a quantitative and/or functional deficiency of a single coagulation protein. Deficiency of coagulation Factors VIII (hemophilia A) and IX (hemophilia B) are the two most common inherited bleeding disorders. The total overall number of hemophilia A and B patients worldwide is approximately 400,000; however, only about ¼ (100,000) of these individuals are treated. Hemophilia A and B can be further divided in regard to the extent of factor deficiency. Mild hemophilia is 5-40% of normal factor levels and represents approximately 25% of the total hemophilia population. Moderate hemophilia is 1-5% of normal factor levels and represents approximately 25% of the total hemophilia population. Severe hemophilia is <1% of normal factor levels and represents approximately 50% of the total hemophilia population and the highest users of currently available therapies.

Since the discovery of cryoprecipitation by Pool (Pool et al., “High-potency antihaemophilic factor concentrate prepared from cryoglobulin precipitate”, Nature, vol. 203, p. 312 (1964)), treatment of these life threatening deficiencies has focused on factor replacement, with a continued effort directed toward improvement in the quality of the Factor VIII and IX concentrates. The most significant improvement has been the availability of recombinant forms of Factors VIII and IX. These highly purified recombinant molecules have a safety and efficacy profile that has made them the primary form of replacement factors used for the treatment of hemophilia. The majority of mild and moderate patients are treated “on demand”, that is when a bleed occurs. Approximately 50-60% of severe patients are treated “on demand”, while the remainder of this population uses prophylactic therapy, which involves administering intravenous factor 2-3 times weekly.

Unfortunately, recombinant factors still retain some of the limitations of concentrates and more highly purified plasma derived factors. These limitations include the relatively short half-life of the molecules, which require frequent injection to maintain effective plasma concentration; high cost; and the development of antibody responses, especially to Factor VIII, in a subpopulation of patients called inhibitor patients.

In a majority of patients who develop inhibitory antibodies, the antibody is only transient. In those patients with a sustained antibody response (˜15%), some respond to complex and expensive tolerization protocols. Those who do not respond to tolerization (˜5-10%) require the use of non-Factor VIII/Factor IX products to control bleeding. Prothrombin Complex Concentrations (PCC), Factor Eight Inhibitor Bypass Agent (FEIBA) and recombinant Factor VIIa (NovoSeven®, FVIIa) are effective Factor VIII/Factor IX bypass treatments for inhibitor patients.

Recombinant Factor VIIa (rFVIIa) treatment is the most used of these bypass agents. Factor VIIa complexes with endogenous tissue factor to activate the extrinsic pathway. It also can directly activate Factor X. The response to rFVIIa treatment is variable. The variable response, along with the poor pharmacokinetic (PK) profile of rFVIIa, can require multiple injections to control bleeding and significantly limits its utility for prophylactic treatment.

A major effort is currently underway towards development of modified Factor VIII, IX and VIIa molecules with improved potency, stability and circulating half-life. It should be noted that in all instances, the products represent incremental improvements to stability, pharmacokinetics and/or formulation of existing replacement factors.

The tissue factor/VIIa (extrinsic) pathway provides for rapid formation of low levels of thrombin that can serve as the initial hemostatic response to initiate and accelerate the Factor VIII, V and IX dependent intrinsic pathway. Tissue factor, Factor VIIa and Factor Xa have a central role in this pathway and it is closely regulated by an endothelial cell associated Kunitz Type proteinase inhibitor, tissue factor pathway inhibitor (TFPI).

Tissue factor pathway inhibitor is a 40 kDa serine protease inhibitor that is synthesized in and found bound to endothelial cell surfaces (“surface TFPI”), in plasma at a concentration of 2-4 nM (“plasma TFPI”) and is stored (200 pM/10⁸ platelets) and released from activated platelets. Approximately 10% of plasma TFPI is unassociated, while 90% is associated with oxidized low density lipoprotein (“LDL”) particles and is inactive. There are two primary forms of TFPI: TFPIα and TFPIβ.

TFPIα contains 3 Kunitz decoy domains, K1, K2 and K3. K1 and K2 mimic protease substrates and inhibit by tight but reversible binding to the target proteases. In the case of TFPIα, K1 binds to and inhibits tissue factor/VIIa, while K2 binds to and inhibits Factor Xa. The role for K3 is unknown at this time, but it may have a role in cell-surface binding and enhancing the inhibition of Factor Xa by K2. TFPIα has a basic C-terminal tail peptide that is the membrane binding site region for the molecule. It is estimated that 80% of the surface TFPI is TFPIα. TFPIα is primarily bound to the endothelial surface associated with the membrane proteoglycans. Heparin has been shown to release TFPIα from cultured endothelium, isolated veins and following intravenous (IV) heparin (unfractionated and LMWH) injection. The exact nature of the release mechanism is unclear (competition or induced release), but TFPI levels can be increased 3-8 fold following IV heparin administration. Some TFPIα can also be found bound to glycosylated phosphatidylinositol (GPI) via an unidentified co-receptor.

TFPIβ is an alternatively spliced version of TFPI that is post-translationally modified with a glycosylated phosphatidylinositol (GPI) anchor. It is estimated that it represents about 20% of the surface TFPI in cultured endothelial cells. Although it has in vitro inhibitory activity, the functional in vivo role is less clear.

Surface TFPI may have a more important role in regulation of coagulation based on its localization to the site of vascular injury and thrombus formation. Surface TFPI represents the largest proportion of active TFPI. Data from several laboratories suggest that TFPI can also have complementary/synergistic effects via interactions with antithrombin III (ATIII) and protein C. TFPI binds to Factor VIIa and Factor Xa via its K1 and K2 domains and to proteoglycans via its K3 and C-terminal domains. The fact that TFPI has a key role in the inhibition of both tissue factor/VIIa and Xa suggests that TFPI inhibition could provide a single treatment or an adjuvant treatment that is given in addition to or combined with recombinant purified factors. An approach to promote a prothrombotic state could be via the upregulation of the tissue factor mediated extrinsic pathway of coagulation. It has been suggested that inhibition of TFPI might improve coagulation in the hemophilia patient.

Studies have demonstrated that TFPI deficiency in mice can increase thrombus formation, and that TFPI antibodies improve bleeding times in Factor VIII deficient rabbits and shorten clotting in plasma from hemophilia patients. In the rabbit, transient hemophilia A was induced by treating rabbits with a Factor VIII antibody. This was followed by treatment with either Factor VIII replacement or an antibody specific to rabbit TFPI. The anti-TFPI treatment produced a reduction in bleeding and a correction of coagulation that was similar to that observed with Factor VIII replacement. Liu et al. (Liu et al., “Improved coagulation in bleeding disorders by Non-Anticoagulant Sulfated Polysaccharides (NASP)”, Thromb. Haemost., vol. 95, pp. 68-76 (2006)) reported the effects of a non-anticoagulant polysaccharide isolated from brown algae that inhibits TFPI. A subsequent paper by Prasad et al. (Prasad et al., “Efficacy and safety of a new-class of hemostatic drug candidate, AV513, in dogs with hemophilia A”, Blood, vol. 111, pp. 672-679 (2008)) also assessed this polysaccharide in hemophilia A dogs. In both studies, it was found that TFPI inhibition had a positive effect on restoration of a normal coagulation profile and, in the dog model, an improvement in hemostatic profile, including an improved thromboelastogram (TEG) and a reduction in nail bleeding time. These data suggest that inhibition of TFPI could provide an approach to treating hemophilia.

Accordingly, it would be beneficial to identify novel therapies for antagonizing TFPI in the treatment of bleeding disorders, or that are used in conjunction with medical procedures, or that are used in combination with another drug or another therapy to induce a pro-coagulant state. The present invention provides materials and methods to meet these and other needs.

### BRIEF SUMMARY OF THE INVENTION

In one aspect, the present invention provides a method of increasing plasma TFPI protein levels in a subject, the method comprising administering BAX499 to the subject. In a further aspect, the subject has a clotting disorder.

In a further aspect and in accordance with the above, the administered BAX499 modulates binding of TFPI to LRP-1.

In a still further aspect and in accordance with any of the above, BAX499 reduces TFPI clearance, thereby increasing TFPI half-life.

In a still further aspect and in accordance with any of the above, the present invention provides a method of increasing procoagulant activity in a subject, the method comprising administering BAX499 to the subject at a dose effective for increasing procoagulant activity. In a further embodiment, BAX499 is administered in a dose in a range of from about 3 mg to about 72 mg. In a still further embodiment, the dose is in a range of from about 5 nM to about 1000 nM.

In a still further aspect and in accordance with any of the above, the present invention provides a method of determining a pharmaceutically effective dose of BAX499, the method comprising assaying blood clotting in samples obtained from subjects administered with differing doses of BAX499. In further embodiments, the subjects are hemophilia patients. In still further embodiments, the subjects have been infused with FVIII prior to, simultaneously with, or subsequent to administration with BAX499. In yet further embodiments, the assaying comprises a member selected from: assaying clotting time, assaying clot stability, and assaying clot size.

In a still further aspect and in accordance with any of the above, the present invention provides a method of administering a pharmaceutically effective dose of BAX499. In further embodiments, the dose is in a range of from about 3 mg to about 72 mg. In a still further embodiment, the dose is in a range of from about 5 nM to about 1000 nM. In a yet further embodiment, the dose of BAX499 does not increase TFPI mRNA levels in said subject.

In a still further aspect and in accordance with any of the above, the present invention provides a method of increasing the procoagulant effect of FVIII in a subject, the method comprising co-administering BAX499 and FVIII to the subject. In further embodiments, the co-administered FVIII is recombinant FVIII. In still further embodiments, the increase in the procoagulant effect is about 1.5 to about 7-fold higher than the increase seen with FVIII alone.

In a still further aspect and in accordance with any of the above, the present invention provides a method of increasing anticoagulant activity in a subject, the method comprising administering BAX499 to the subject at a dose effective for increasing anticoagulant activity.

In a still further aspect and in accordance with any of the above, the present invention provides a method of determining whether a molecule inhibits or activates TFPI protein function, a method of determining whether a molecule increases or decreases intracellular or membrane bound TFPI plasma protein levels, or a method of determining whether a molecule increases or decreases TFPI clearance. In these embodiments, the methods of determining comprise conducting a competitive assay with the molecule and BAX499. In a further embodiment, the competitive assay is a competitive binding assay. In a still further embodiment, the molecule is a member selected from an aptamer, a peptide, an antibody, and a small molecule. In a further embodiment, the molecule inhibits TFPI protein function. In a further embodiment, the molecule activates TFPI protein function. In a further embodiment, the molecule increases intracellular or membrane bound TFPI plasma protein levels. In a further embodiment, the molecule decreases intracellular or membrane bound TFPI plasma protein levels. In a further embodiment, the molecule increases TFPI clearance. In a further embodiment, the molecule decreases TFPI clearance.

### BRIEF DESCRIPTION OF THE DRAWINGS

FIG. 1 illustrates immuno-precipitation of plasma TFPI by anti-TFPI antibodies and biotinylated anti TFPI aptamer.

FIG. 2 illustrates the biomolecular interaction analysis (BiaCore) of fl-TFPI binding to biotinylated aptamer.

FIG. 3 illustrates release of cellular TFPI upon incubation with BAX 499.

FIG. 4 illustrates release of cellular TFPI upon incubation with BAX 499.

FIG. 5 illustrates release of cellular TFPI upon incubation with BAX 499.

FIG. 6 illustrates a schematic representation of the HUVE cell-based FX activation assay.

FIG. 7 illustrates the impact of BAX 499 on HUVE cell-based FX.

FIG. 8 illustrates the impact of BAX 499 on total cell surface TFPI activity in a HUVE cell-based FX assay.

FIG. 9 illustrates the impact of BAX 499 on TFPI gene expression as quantified by real time PCR.

FIG. 10 illustrates cell surface TFPI by Fluorescence-activated cell sorting (FACS) analysis of non-permeabilized cells and cell supernatant TFPI by ELISA.

FIG. 11 illustrates total cellular TFPI by FACS analysis.

FIG. 12 illustrates biomolecular interaction analysis (BiaCore) of fl-TFPI binding to biotinylated LRP.

FIG. 13 illustrates the pharmacokinetics of human fl-TFPI in mice.

FIG. 14 illustrates a time course of TFPI digestion by human neutrophil elastase in the absence and presence of BAX 499 (1 μM); and the FXa inhibitory activity of TFPI.

FIG. 15 illustrates an FXa inhibition assay and extrinsic tenase assay.

FIG. 16 illustrates an FXa inhibition assay and extrinsic tenase assay.

FIG. 17 illustrates an FXa inhibition assay and extrinsic tenase assay.

FIG. 18 illustrates thrombin generation of FVIII inhibited plasma in presence of BAX 499 (1000 nM) at increasing fl-TFPI (up to 5 nM).

FIG. 19 illustrates inhibition of elevated plasma concentrations of human fl-TFPI by BAX 499 (10-1000 nM) in a thrombin generation assay in FVIII-inhibited plasma.

FIG. 20 illustrates BAX 499 requirement for neutralization of elevated fl-TFPI.

FIG. 21 illustrates clot time following addition of fl-TFPI to FVIII inhibited whole blood in the absence of BAX499.

FIG. 22 illustrates clot time following addition of fl-TFPI to FVIII inhibited whole blood in the absence of BAX499.

FIG. 23 illustrates the full-length (A) and total (B) plasma TFPI concentrations (nM) for Group 1 monkeys in non-human primate model.

FIG. 24 illustrates the full-length (A) and total (B) plasma TFPI concentrations (nM) for Group 2 monkeys in non-human primate model.

FIG. 25 illustrates the full-length (A) and total (B) plasma TFPI concentrations (nM) for Group 3 monkeys in non-human primate model.

FIG. 26 illustrates the full-length (A) and total (B) plasma TFPI concentrations (nM) for Group 4 monkeys in non-human primate model.

FIG. 27 illustrates the full-length (A) and total (B) plasma TFPI concentrations (nM) for monkey 1006-M in non-human primate model.

FIG. 28 illustrates the full-length (A) and total (B) plasma TFPI concentrations (nM) for monkey 1005-M in non-human primate model.

FIG. 29 illustrates the full-length (A) and total (B) plasma TFPI concentrations (nM) for monkey 1505-F in non-human primate model.

FIG. 30 illustrates the full-length (A) and total (B) plasma TFPI concentrations (nM) for monkey 1506-F in non-human primate model.

FIG. 31 illustrates the full-length (A) and total (B) plasma TFPI concentrations (nM) for monkey 2005-M in non-human primate model.

FIG. 32 illustrates the full-length (A) and total (B) plasma TFPI concentrations (nM) for monkey 2006-M in non-human primate model.

FIG. 33 illustrates the full-length (A) and total (B) plasma TFPI concentrations (nM) for monkey 2505-F in non-human primate model.

FIG. 34 illustrates the full-length (A) and total (B) plasma TFPI concentrations (nM) for monkey 2506-F in non-human primate model.

FIG. 35 illustrates the full-length (A) and total (B) plasma TFPI concentrations (nM) for monkey 3005-M in non-human primate model.

FIG. 36 illustrates the full-length (A) and total (B) plasma TFPI concentrations (nM) for monkey 3106-M in non-human primate model.

FIG. 37 illustrates the full-length (A) and total (B) plasma TFPI concentrations (nM) for monkey 3505-F in non-human primate model.

FIG. 38 illustrates the full-length (A) and total (B) plasma TFPI concentrations (nM) for monkey 3506-F in non-human primate model.

FIG. 39 illustrates the full-length (A) and total (B) plasma TFPI concentrations (nM) for monkey 4005-M in non-human primate model.

FIG. 40 illustrates the full-length (A) and total (B) plasma TFPI concentrations (nM) for monkey 4006-M in non-human primate model.

FIG. 41 illustrates the full-length (A) and total (B) plasma TFPI concentrations (nM) for monkey 4505-M in non-human primate model.

FIG. 42 illustrates the full-length (A) and total (B) plasma TFPI concentrations (nM) for monkey 4506-F in non-human primate model.

FIG. 43 illustrates a graphical representation of BAX 499 levels over time in days from placebo and BAX 499 treated subject blood samples.

FIG. 44 illustrates a graphical representation of TFPI level over time in days from placebo and BAX 499 treated subject blood samples.

FIG. 45 illustrates the tight correlation between TFPI levels and BAX 499 concentration.

FIG. 46 illustrates BAX 499 regulation of TFPI inhibition of FXa. A. Time courses of active FXa remaining after addition of 3 nM TFPI to 1 nM FXa in the presence of BAX499. ♦, no aptamer; ▴, 2.5 nM aptamer; , 5 nM aptamer; ▪, 10 nM aptamer; Δ, 25 nM aptamer; , 100 nM aptamer; *, 250 nM aptamer. B. Dissociation constant (Kd) calculated for each concentration of aptamer.

FIG. 47 illustrates BAX 499 regulation of TFPI inhibition of TF/FVIIa. Fluorogenic activity of 1 nM TF/VIIa in the presence of 12 nM TFPI and varying BAX499 concentrations.

FIG. 48 illustrates BAX 499 regulation of TFPI inhibition of the extrinsic FXase. Initial rates of FXa generation in the presence of 20 pM TF/FVIIa, 250 nM FX, 20 μM phospholipid and 3 nM TFPI at varying aptamer concentrations (8.33, 25, 50, 200, 500, 1000 nM). The dotted horizontal line gives the rate for the experiment in the absence of TFPI (±aptamer).

FIG. 49 illustrates BAX 499 modulation of thrombin generation in a synthetic coagulation proteome model of sever hemophilia A and B. 5 pM TF-initiated thrombin generation in a purified system at varying BAX499 concentrations. A. Control. B. Hemophilia A. C. Hemophilia B. ⋄ no aptamer (0% FVIII (panel B) or 0% FIX (panel C)); ♦, no aptamer (100% FVIII and FIX); ▴, 1 nM aptamer; , 5 nM aptamer; ▪, 10 nM aptamer; +, no TFPI, no aptamer.

FIG. 50 illustrates BAX 499 modulation of thrombin generation in synthetic coagulation proteome models of mild, moderate, and severe hemophilia A. Thrombin generation was initiated with 5 pM TF in the absence (panel A) and presence (panel B) of 2.5 nM BAX499. ♦, 100% FVIII (No aptamer); X, 100% FVIII (2.5 nM aptamer); ▪, 40% FVIII; , 5% FVIII; ▴, 2% FVIII; ⋄0% FVIII.

FIG. 51 illustrates BAX 499 modulation of thrombin generation in contact pathway inhibited plasma. A. Normal plasma B. Induced hemophilia B plasma. Solid black line, no BAX499; broken black line, 1 nM BAX499; dotted black line 10 nM BAX499; solid gray line, 100 nM BAX499; broken gray line, 500 nM BAX499; dotted gray line 1000 nM BAX499.

FIG. 52 illustrates BAX 499 effects on thrombin generation in contact pathway inhibited whole blood. A. Subject 1. B. Subject 2. Arrows indicate visual clotting times. ♦, Control; ▴, Control +100 nM aptamer; ⋄, Induced hemophilia B; Δ, Induced hemophilia B +100 nM aptamer.

FIG. 53 illustrates thromboelastographic analysis of BAX 499 effects on clot stability. A-C. Subjects 3-5. Each subject's contact pathway inhibited blood supplemented with 1 nM t-PA was analyzed in the presence (gray lines) or absence (black lines) of 100 nM BAX499. Both control (outer lines) and induced hemophilia B (inner lines) are shown.

FIG. 54 illustrates the effects of BAX499 and factor VIII on spatial clotting in hemophilia A. (a) Typical light-scattering time-lapse images of clot growth in plasma initiated by immobilized TF at surface density of 2 pmole/m²: hemophilia A before and after factor VIII administration, hemophilia A with addition of BAX499 100 nM. TF-coated activator is seen as a vertical black strip on the left side of each image. White bar shows the scale of 0.5 mm. (b) Clot size versus time plots for the experiments shown in (a). The panel also illustrates parameters used for experiment analysis throughout the study: lag time (time of clot growth initiation); α, initial velocity (mean slope over the first 10 min); β, stationary velocity (mean slope over the following 30 min); clot size after 60 min of the experiment.

FIG. 55 illustrates the efficiency of BAX499 in hemophilia A plasmas prepared with different methodologies. Ratios of clotting parameter with or without 300 nM BAX499 for freshly prepared plasma collected into CTI and the same frozen\thawed plasma. The error bars were calculated based on S.E.

FIG. 56 illustrates the characterization of hemophilia A patients. Activity of factor VIII and APTT for all patient blood samples throughout the experiments.

FIG. 57 illustrates the effect of BAX499 on clotting in hemophilia A plasma for patient with relative BAX499 effect dependent on fVIII:C activity. The panels show clotting parameters for hemophilia A plasma supplemented with BAX499: (a) lag time, (b) initial velocity, (c) stationary velocity, (d) clot size. The data are means±S.E., the number of measurements is n=2.8.

FIG. 58 illustrates the effect of BAX499 on clotting in hemophilia A plasma for patient with relative BAX499 effect independent on fVIII:C activity. The panels show clotting parameters for hemophilia A plasma supplemented with BAX499: (a) lag time, (b) initial velocity, (c) stationary velocity, (d) clot size. The data are means±S.E., the number of measurements is n=2.5.

FIG. 59 illustrates a drug-drug interaction of the combined effects of BAX499 and factor VIII on the size of clots formed in hemophilia A. The panels illustrate clot sizes at 60 min after the beginning of the experiment dependence on measured activity of factor VIII and BAX499 concentration. Patients from 1 (a) to 9 (i) are shown.

FIG. 60 illustrates the dependence of clotting parameters on the BAX499 concentration in hemophilia A plasma for different time points of factor VIII:C pharmacokinetics. Averaged clot formation parameters dependence on BAX499 concentrations: (a) lag time, (b) initial velocity, (c) stationary velocity, (d) clot size. The data are means±S.E., the number of patients is n=9.

FIG. 61 illustrates the maximal effect of BAX499 in hemophilia A plasma for three different ranges of factor VIII:C. Comparison of BAX499 effect in different ranges of factor VIII activity: (a) lag time, (b) initial velocity, (c) stationary velocity, (d) clot size. The data are means S.E., the number of statistical averaging is n=5.11.

FIG. 62 illustrates the mechanisms of drug-drug interaction: relative contribution of extrinsic and intrinsic tenases to clotting. (A) Clot size as a function of time for four combinations of factor VIII and TFPI deficiencies. (B) Concentration of factor Xa produced by either intrinsic or extrinsic tenase as a function of space and time for the same experiments. The data were obtained in computer simulations using a mathematical model of clotting initiated by immobilized TF at 10 pmole/m2.

FIG. 63 illustrates the two types of patients of Example 11.

FIG. 64 illustrates the effect of lag time from a new solution of BAX 499 that was stored dry for 2 years compared to commercially available plasma.

FIG. 65 illustrates the effect of clot size from a new solution of BAX 499 that was stored dry for 2 years compared to commercially available plasma.

FIG. 66 illustrates the effect of BAX 499 (300 nM) on spatial clot formation in commercially available factor VIII-deficient plasma.

FIG. 67 illustrates (A) the ROTEM principle and (B) analysis of the Overall Fibrinolysis Potential (OFP). The area under curve (AUC) of the ROTEM® tracings of lysis induced blood and normal blood is calculated as an integral function of the curve from the clot time (CT) (amplitude of 5 mm) to 6000 s reflecting the Overall Hemostasis Potential (OHP) and the Overall Coagulation Potential (OCP), respectively. The OFP was calculated as a percentage of the difference between the OHP and OCP: OFP=(OCP−OHP)/OCP×100.

FIG. 68 illustrates procoagulant activity of BAX 499 in ΔFVIII human blood. (A) ROTEM® tracings and (B) table of the parameters of the titration of BAX 499 (5, 50, 500, 1000 nM) in ΔFVIII blood and normal whole blood (NB) control at very low TF trigger (44 fM).

FIG. 69 illustrates the effect of BAX 499 in ΔFVIII blood w/o and with addition of tPA for induction of fibrinolysis. AUC was calculated from ROTEM® tracings of ΔFVIII and ΔFVIII blood with 1 μM BAX 499 without tPA (OCP) (A) or after addition of 90 ng/ml tPA as an inducer of fibrinolysis (OHP) (B). Normal blood is shown as control. The values of OCP, OHP and the calculated OFP are shown in the table (C). The difference of the values of the reactions with or without BAX 499 is expressed as ‘fold increase’.

FIG. 70 illustrates EPT and peak thrombin values plotted as a function of FVIII concentration in CAT assay.

FIG. 71 illustrates EPT and peak thrombin values plotted as a function of FVIII concentration in CAT assay.

FIG. 72 illustrates representative thrombin generation curves with FVIII alone in CAT assay.

FIG. 73 illustrates FVIII standard curve using peak thrombin values from CAT assay.

FIG. 74 illustrates FVIII equivalent activities as a function of FVIII concentration based on peak thrombin values from the CAT assay.

FIG. 75 illustrates FVIII standard curve based on clot time values from the ROTEM assay.

FIG. 76 illustrates FVIII equivalent activities (EA) of 2000 nM BAX 499 in combination with different concentration of FVIII in the ROTEM assay.

FIG. 77 illustrates representative ROTEM traces with FVIII alone and in combination with 2000 nM BAX 499.

FIG. 78 illustrates a description of ROTEM principle.

FIG. 79 illustrates general ROTEM tracing.

### DETAILED DESCRIPTION OF THE INVENTION

### Overview

The present invention provides compositions and methods for modulating TFPI protein function and/or TFPI protein plasma concentration. Such modulation can in some aspects be used to treat blood disorders such as bleeding disorders and clotting disorders.

In one aspect, the present invention provides methods and compositions for inhibiting TFPI protein function, particularly inhibiting TFPI's effects as an anticoagulant. In further embodiments, aptamers such as BAX499 (SEQ ID NO: 1) are administered to a subject to inhibit TFPI. In specific embodiments, BAX499 is administered to subjects in an amount effective to inhibit TFPI's anticoagulant activity. In further embodiments, the subjects receiving BAX499 suffer from a bleeding disorder such as Hemophilia A. In still further embodiments, BAX499 is administered in a dose ranging from about 1 mg to about 100 mg. In yet further embodiments, the BAX499 is administered in a dose ranging from about 1 nM to about 2000 nM.

In a further aspect, the present invention provides methods for increasing the plasma concentration of TFPI protein in a subject by administering a TFPI modulator to the subject. In some embodiments, such methods include administering an aptamer to the subject. In an exemplary embodiment, the aptamer is BAX499. In further embodiments, the increase in TFPI protein plasma levels is not accompanied by an increase in TFPI mRNA levels.

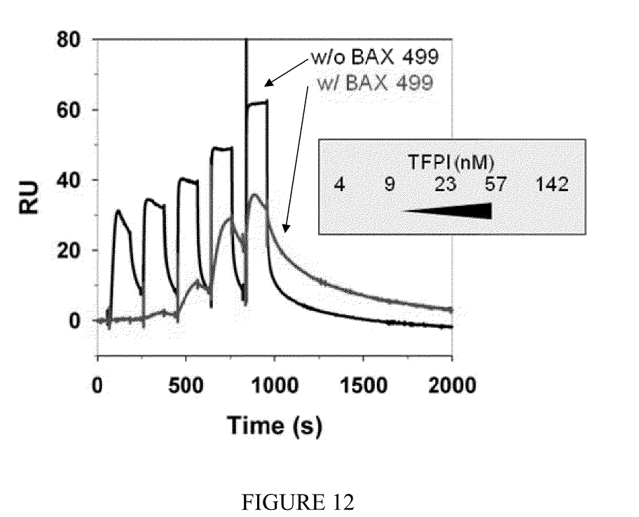

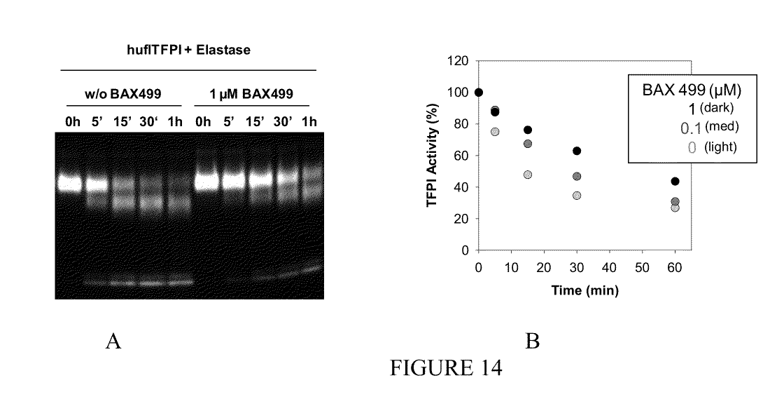

In further embodiments, BAX499 increases TFPI protein plasma levels by interfering with TFPI clearance. In still further embodiments, BAX499 interferes with TFPI clearance by disrupting TFPI binding to the receptor LRP-1 (for example, see FIG. 12). In yet further embodiments, BAX499 increases TFPI protein plasma levels by delaying proteolytic degradation of TFPI (see FIG. 14).

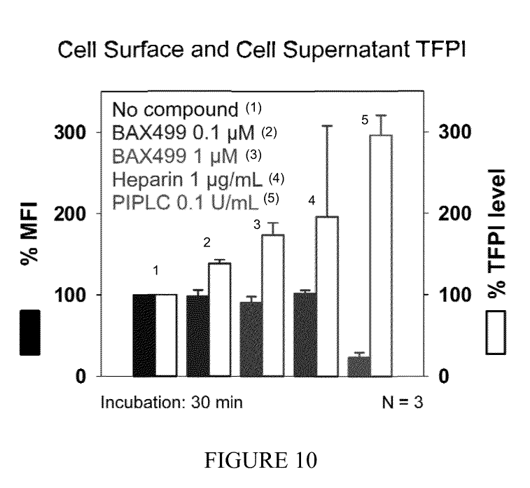

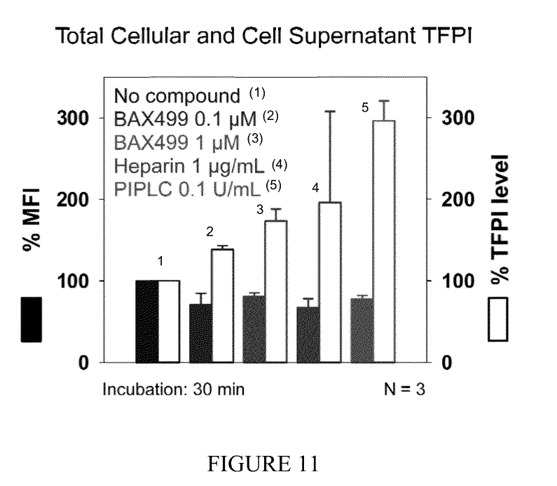

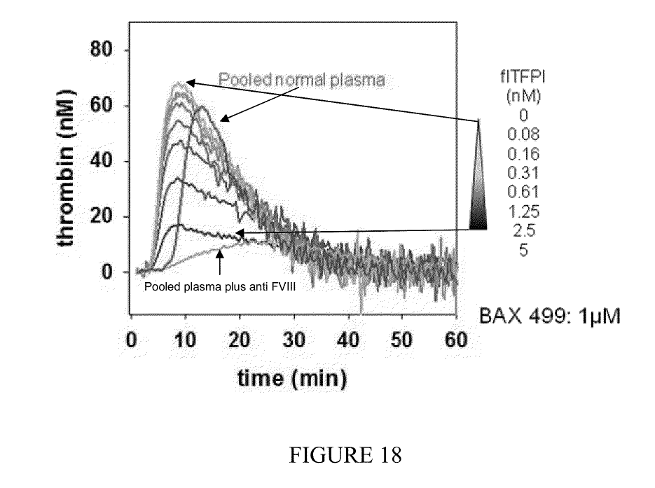

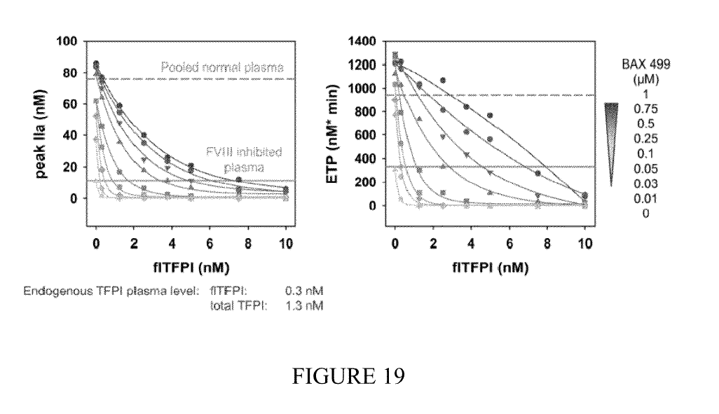

In a still further aspect, the present invention provides methods and compositions for modulating a homeostasis between concentrations of BAX499 and TFPI protein plasma levels in a subject. BAX499 affects both TFPI protein function and TFPI plasma levels. Administering BAX499 inhibits TFPI protein function, as demonstrated in assays that include without limitation measurements of clotting time (such as activated thromboplastin time (aPTT) assay, dilute prothrombin time (PT) assay, and assay of fibrinogen levels), measurements of hemostasis (such as Rotational Elastometry (ROTEM) and Thromboelastography (TE)), measurement of clot stability, and measurement of clot size. Although lower concentrations of BAX499 effectively inhibit TFPI protein function, as increasing amounts of BAX499 are administered to a subject and/or build up in the plasma, there is a concomitant increase in TFPI protein plasma levels in the subject. This increase in TFPI protein plasma levels seems to be, without being limited by theory, due to release of TFPI protein from intracellular stores rather than release of membrane-bound TFPI (see FIGS. 10-11 and Example 4). BAX499 inhibition of TFPI function was more effective in the patient population studied when TFPI protein plasma levels were close to physiological levels (e.g., around 1.3 nm—see FIGS. 18-19 and Example 8) than at higher TFPI protein plasma levels (e.g., 5 nm or higher, see FIGS. 18-20).

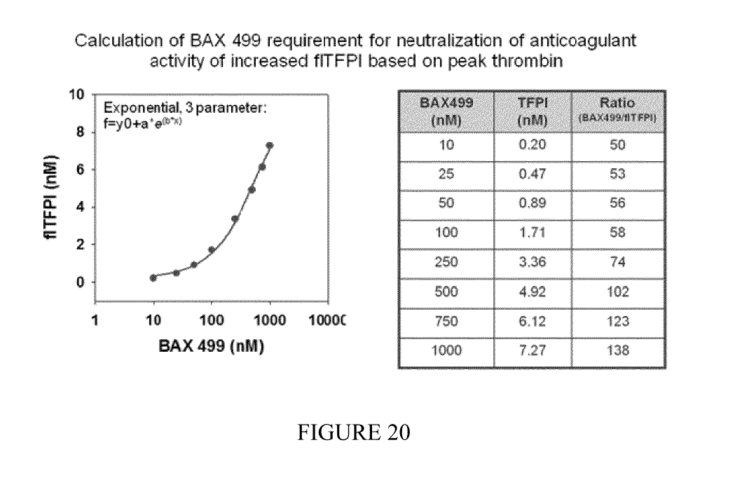

As shown in FIG. 20, the concentration of BAX499 needed to inhibit TFPI's anticoagulant effect increases exponentially: an approximately 50-fold excess of BAX 499 neutralizes 0.2 nM TFPI added to FVIII-inhibited plasma whereas a 140-fold excess is required for neutralization of 7.3 nM additional full-length TFPI (“fl-TFPI”). Thus, a pair of BAX 499 and TFPI concentrations which would be under the fitted line (FIG. 20) would support procoagulant activity of BAX 499, whereas a pair above the fitted line would result in a net anticoagulant effect. As will be appreciated, fine tuning the concentrations of BAX499 and TFPI protein in this way allows control of the use of BAX499 as a procoagulant to treat bleeding disorders or as an anticoagulant to treat clotting disorders.

In a still further aspect, the present invention provides a method of determining a pharmaceutically effective dose of a TFPI modulator for promoting coagulation in a subject. In some embodiments, the method includes measuring clotting time, clot size, and clot stability from samples obtained from subjects provided varying doses of the TFPI modulator. Such dose response analyses provide a way to identify the optimal concentration of the TFPI modulator. In exemplary embodiments, the TFPI modulator is an aptamer. In further embodiments, the modulator is BAX499.

In a yet further aspect, the present invention provides methods and compositions for identifying a molecule (i.e., a “test” molecule) that inhibits or activates TFPI protein function, increases or decreases intracellular or membrane bound TFPI protein plasma levels, or increases or decreases TFPI clearance. In some embodiments, the determining measures TFPI protein function. In some embodiments, the determining measures intracellular or membrane bound TFPI plasma concentration. In some embodiments, the determining measures TFPI clearance. In certain embodiments, the methods of the invention include conducting a competitive assay between the molecule and BAX499. In still further embodiments, the competitive assay is a competitive binding assay. In yet further embodiments, the test molecule is a member selected from an aptamer, a peptide, an antibody, and a small molecule. In certain embodiments, the molecule inhibits TFPI protein function. In certain embodiments, the molecule activates TFPI protein function. In certain embodiments, the molecule increases intracellular or membrane bound TFPI plasma protein levels. In certain embodiments, the molecule decreases intracellular or membrane bound TFPI plasma protein levels. In certain embodiments, the molecule increases TFPI clearance. In certain embodiments, the molecule decreases TFPI clearance.

In a further aspect, administering BAX499 affects and, in some embodiments, complements, the procoagulant effects of co-administered FVIII. The FVIII may be administered prior to, subsequent to, or simultaneously with, BAX499. In further embodiments, the administration of BAX499 together with FVIII reduces the amount of FVIII needed to increase coagulation—in other words, BAX499 together with FVIII has the equivalent effect on coagulation as a higher concentration of FVIII (see Table 11 in the Examples section herein). In further embodiments, BAX499 increases the procoagulant effect of FVIII by from about 1.5 fold to about 10 fold higher as compared to when FVIII is administered alone.

### DEFINITIONS

As used herein, “aptamer” refers to an isolated or purified nucleic acid that binds with high specificity and affinity to a target, such as a protein, through interactions other than Watson-Crick base pairing.

As used herein, “TFPI” and “flTFPI” refer to tissue factor pathway inhibitor and full length tissue factor pathway inhibitor respectively.

As used herein, “TFPI protein function” refers to any in vivo or in vitro function, including TFPI's inhibition of both tissue factor/VIIa and Xa and TFPI's effect as an anticoagulant.

As used herein, “TFPI clearance” refers to the physiological process of removing TFPI from an organism, such as by diffusion, exfoliation, removal via the bloodstream, and excretion in urine, or via sweat or other fluid.

As discussed herein, a “modulator” of TFPI protein function refers to any molecule that inhibits or stimulates the activity of TFPI, increases or decreases TFPI plasma protein levels, or increases or decreases TFPI clearance.

As used herein, the term “factor VIII” or “FVIII” refers to any form of factor VIII molecule with the typical characteristics of blood coagulation factor VIII, whether endogenous to a patient, derived from blood plasma, or produced through the use of recombinant DNA techniques, and including all modified forms of factor VIII. Factor VIII (FVIII) exists naturally and in therapeutic preparations as a heterogeneous distribution of polypeptides arising from a single gene product (see, e.g., Andersson et al., Proc. Natl. Acad. Sci. USA, 83:2979-2983 (1986)). Commercially available examples of therapeutic preparations containing Factor VIII include those sold under the trade names of HEMOFIL M, ADVATE, and RECOMBINATE (available from Baxter Healthcare Corporation, Deerfield, III, U.S.A.).

As used herein, “rFVIII” refers to recombinant FVIII.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

As used herein, the term “bleeding disorder” refers to any disorder that leads to poor blood clotting and continuous bleeding. Also referred to as a coagulopathy, such disorders include for example hemophilia.

As used herein, the term “clotting disorder” refers to any disorder in which there is a tendency toward excessive clotting, including without limitation thrombosis.

As used herein, the terms “hemophilia” or “haemophilia” refer to a group of disease states broadly characterized by reduced blood clotting or coagulation. Hemophilia may refer to Type A, Type B, or Type C hemophilia, or to the composite of all three diseases types. Type A hemophilia (hemophilia A) is caused by a reduction or loss of factor VIII (FVIII) activity and is the most prominent of the hemophilia subtypes. Type B hemophilia (hemophilia B) results from the loss or reduction of factor IX (FIX) clotting function. Type C hemophilia (hemophilia C) is a consequence of the loss or reduction in factor XI (FXI) clotting activity. Hemophilia A and B are X-linked diseases, while hemophilia C is autosomal. Common treatments for hemophilia include both prophylactic and on-demand administration of clotting factors, such as FVIII, FIX, including Bebulin®-VH, and FXI, as well as FEIBA-VH, desmopressin, and plasma infusions.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. The term “purified” in some embodiments denotes that a nucleic acid or protein gives rise to essentially one band in an electrophoretic gel. In other embodiments, it means that the nucleic acid or protein is at least 50% pure, more preferably at least 60%, 65%, 70%, 75%, 80%, 85%, 90%, 95%, 96%, 97%, 98%, 99% or more pure. “Purify” or “purification” in other embodiments means removing at least one contaminant from the composition to be purified. In this sense, purification does not require that the purified compound be homogenous, e.g., 100% pure.

As used herein, “administering” (and all grammatical equivalents) includes intravenous administration, intramuscular administration, subcutaneous administration, oral administration, administration as a suppository, topical contact, intraperitoneal, intralesional, or intranasal administration, or the implantation of a slow-release device, e.g., a mini-osmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “therapeutically effective amount or dose” or “therapeutically sufficient amount or dose” or “effective or sufficient amount or dose” or “pharmaceutically effective amount or dose” refer to a dose that produces therapeutic effects for which it is administered. For example, a therapeutically effective amount of a drug useful for treating hemophilia can be the amount that is capable of preventing or relieving one or more symptoms associated with hemophilia. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

By “subject” is meant any member of the subphylum chordata, including, without limitation, humans and other primates, including non human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are of interest.

The term “patient,” is used in its conventional sense to refer to a living organism suffering from or prone to a condition that can be prevented or treated by administration of a composition of the invention, and includes both humans and non-human species. The terms “patient” and “subject” are used interchangeably throughout the application, and, as discussed above, these terms include both human and veterinary subjects.

As used herein, the term “about” denotes an approximate range of plus or minus 10% from a specified value. For instance, the language “about 20%” encompasses a range of 18-22%.

As used herein, the term “half-life” refers to the period of time it takes for the amount of a substance undergoing decay (or clearance from a sample or from a patient) to decrease by half.

As used herein, the term “LRP-1” refers to the low density lipoprotein receptor-related protein, which is also referred to as alpha 2-macroglobulin. This protein mediates the cellular degradation of TFPI.

As used herein, the term “primary nucleotide sequence” refers to the 5′ to 3′ linear sequence of nucleotide bases of the nucleic acid sequence that forms an aptamer, without regard to 3′ or 5′ modifications.

As used herein, the terms “sequence identity” or “% identity”, in the context of two or more nucleic acid or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. For sequence comparison, typically one sequence acts as a reference sequence to which test sequences are compared. Optimal alignment of sequences for comparison can be conducted, e.g., by the local homology algorithm of Smith & Waterman, Adv. Appl. Math. 2: 482 (1981); by the homology alignment algorithm of Needleman & Wunsch, J. Mol. Biol. 48: 443 (1970); by the search for similarity method of Pearson & Lipman, Proc. Nat'l. Acad. Sci. USA 85: 2444 (1988); by computerized implementations of these algorithms (GAP, BESTFIT, FASTA, and TFASTA in the Wisconsin Genetics Software Package, Genetics Computer Group, 575 Science Dr., Madison, Wis.); or by visual inspection (see generally, Ausubel, F. M. et al., Current Protocols in Molecular Biology, pub. by Greene Publishing Assoc. and Wiley-Interscience (1987)). One example of an algorithm that is suitable for determining percent sequence identity is the algorithm used in the basic local alignment search tool (hereinafter “BLAST”), see, e.g. Altschul et al., J. Mol. Biol. 215: 403-410 (1990) and Altschul et al., Nucleic Acids Res., 15: 3389-3402 (1997), which is publicly available through the National Center for Biotechnology Information (hereinafter “NCBI”).

Compositions of the Invention

The present invention provides compositions comprising modulators of TFPI protein plasma levels and TFPI protein function. Modulators of TFPI may include any molecule effective to alter TFPI protein plasma levels or TFPI protein function (including TFPI's anticoagulant effects).

In a specific embodiment, compositions of the invention comprise an aptamer that is able to affect TFPI protein plasma levels and/or TFPI protein function. As used herein, “aptamer” refers to an isolated or purified nucleic acid that binds with high specificity and affinity to a target through interactions other than Watson-Crick base pairing. An aptamer has a three dimensional structure that provides chemical contacts to specifically bind to a target. Unlike traditional nucleic acid binding, aptamer binding is not dependent upon a conserved linear base sequence, but rather a particular secondary or tertiary structure. That is, the nucleic acid sequences of aptamers are non-coding sequences. Any coding potential that an aptamer may possess is entirely fortuitous and plays no role whatsoever in the binding of an aptamer to a target. A typical minimized aptamer is 5-15 kDa in size (15-45 nucleotides), binds to a target with nanomolar to sub-nanomolar affinity, and discriminates against closely related targets (e.g., aptamers will typically not bind to other proteins from the same gene or functional family). Aptamers of use in the invention are described for example in U.S. Ser. No. 12/858,369, filed Aug. 17, 2010 and U.S. Ser. No. 13/026,165, filed Feb. 11, 2011, each of which is hereby incorporated by reference in its entirety for all purposes and in particular for all teachings related to TFPI aptamers and uses thereof.

In further embodiments, the invention includes nucleic acid aptamers, preferably of 20-55 nucleotides in length, that bind to TFPI and which, in some embodiments, functionally modulate, e.g., stimulate, block or otherwise inhibit or stimulate, the activity of TFPI.

The TFPI aptamers of use in the present invention bind at least in part to TFPI or a variant or one or more portions (or regions) thereof. For example, the TFPI aptamers may bind to or otherwise interact with a linear portion or a conformational portion of TFPI. A TFPI aptamer binds to or otherwise interacts with a linear portion of TFPI when the aptamer binds to or otherwise interacts with a contiguous stretch of amino acid residues that are linked by peptide bonds. A TFPI aptamer binds to or otherwise interacts with a conformational portion of TFPI when the aptamer binds to or otherwise interacts with non-contiguous amino acid residues that are brought together by folding or other aspects of the secondary and/or tertiary structure of the polypeptide chain. The TFPI may be from any species, but is preferably human.

The TFPI aptamers preferably comprise a dissociation constant for human TFPI, or a variant thereof, of less than 100 μM, less than 1 μM, less than 500 nM, less than 100 nM, preferably 50 nM or less, preferably 25 nM or less, preferably 10 nM or less, preferably 5 nM or less, more preferably 3 nM or less, even more preferably 1 nM or less, and most preferably 500 pM or less. In some embodiments, the dissociation constant is determined by dot blot titration.

The TFPI aptamers may be ribonucleic acid, deoxyribonucleic acid, modified nucleic acids (for example, 2′-modified) or mixed ribonucleic acid, deoxyribonucleic acid and modified nucleic acids, or any combination thereof. The aptamers may be single stranded ribonucleic acid, deoxyribonucleic acid, modified nucleic acids (for example, 2′-modified), ribonucleic acid and modified nucleic acid, deoxyribonucleic acid and modified nucleic acid, or mixed ribonucleic acid, deoxyribonucleic acid and modified nucleic acids, or any combination thereof.

In some embodiments, the TFPI aptamers comprise at least one chemical modification. In some embodiments, the chemical modification is selected from the group consisting of: a chemical substitution at a sugar position, a chemical substitution at an internucleotide linkage and a chemical substitution at a base position. In other embodiments, the chemical modification is selected from the group consisting of: incorporation of a modified nucleotide; a 3′ cap; a 5′ cap; conjugation to a high molecular weight, non-immunogenic compound; conjugation to a lipophilic compound; incorporation of a CpG motif; and incorporation of a phosphorothioate or phosphorodithioate into the phosphate backbone. In a preferred embodiment, the non-immunogenic, high molecular weight compound is polyalkylene glycol, and more preferably is polyethylene glycol (PEG). In some embodiments, the polyethylene glycol is methoxypolyethylene glycol (mPEG). In another preferred embodiment, the 3′ cap is an inverted deoxythymidine cap.

The modifications described herein may affect aptamer stability, e.g., incorporation of a capping moiety may stabilize the aptamer against endonuclease degradation. Additionally, the modifications described herein may affect the binding affinity of an aptamer to its target, e.g., site specific incorporation of a modified nucleotide or conjugation to a PEG may affect binding affinity. The effect of such modifications on binding affinity can be determined using a variety of art-recognized techniques, such as, e.g., functional assays, such as an ELISA, or binding assays in which labeled trace aptamer is incubated with varying target concentrations and complexes are captured on nitrocellulose and quantitated, to compare the binding affinities pre- and post-incorporation of a modification.

In some embodiments, the TFPI aptamers bind at least in part to TFPI or a variant or one or more portions thereof and act as an antagonist to inhibit the function of TFPI.

In further embodiments, the TFPI aptamers completely or partially inhibit, reduce, block or otherwise modulate TFPI-mediated inhibition of blood coagulation. The TFPI aptamers are considered to completely modulate, block, inhibit, reduce, antagonize, neutralize or otherwise interfere with TFPI biological activity, such as TFPI-mediated inhibition of blood coagulation, when the level of TFPI-mediated inhibition in the presence of the TFPI aptamer is decreased by at least 95%, e.g., by 96%, 97%, 98%, 99% or 100% as compared to the level TFPI-mediated inhibition in the absence of the TFPI aptamer. The TFPI aptamers are considered to partially modulate, block, inhibit, reduce, antagonize, neutralize or otherwise interfere with TFPI biological activity, such as TFPI-mediated inhibition, when the level of TFPI-mediated inhibition in the presence of the TFPI aptamer is decreased by less than 95%, e.g., 10%, 15%, 20%, 25%, 30%, 35%, 40%, 45%, 50%, 55%, 60%, 65%, 70%, 75%, 80%, 85% or 90% as compared to the level of TFPI activity in the absence of the TFPI aptamer.

In certain aspects, the TFPI aptamer used in accordance with the present invention is an aptamer or a salt thereof comprising the following nucleic acid sequence: PEG40K-NH-mG-mG-mA-mA-mU-mA-mU-mA-dC-mU-mU-mG-mG-dC-mU-dC-mG-mU-mU-mA-mG-mG-mU-mG-dC-mG-mU-mA-mU-mA-mU-mA-3T (SEQ ID NO: 1) (also referred to herein as “BAX499”), wherein “NH” is from a 5′-hexylamine linker phosphoramidite, “3T” is an inverted deoxythymidine, “dN” is a deoxynucleotide, “mN” is a 2′-O Methyl containing nucleotide and “PEG” is a polyethylene glycol. In some embodiments, the TFPI aptamer is an aptamer or a salt thereof that consists of the nucleic acid sequence of SEQ ID NO: 1. In some embodiments, the PEG40K moiety of SEQ ID NO: 1 is a branched PEG moiety having a total molecular weight of 40 kDa. In other embodiments, the PEG40K moiety of SEQ ID NO: 1 is a linear PEG moiety having a molecular weight of 40 kDa. In further embodiments, the PEG40K moiety of SEQ ID NO: 1 is a methoxypolyethylene glycol (mPEG) moiety having a molecular weight of 40 kDa. In still further embodiments, the PEG40K moiety of SEQ ID NO: 1 is a branched mPEG moiety that contains two mPEG20K moieties, each having a molecular weight of 20 kDa. In a preferred embodiment, the PEG40K moiety of SEQ ID NO: 1 is a branched PEG40K moiety and is connected to the aptamer through a linker. In one embodiment, the PEG40K moiety is connected to the aptamer using a 5′-amine linker phosphoramidite. In a further embodiment, the PEG40K moiety is an mPEG moiety having a total molecular weight of 40 kDa and is connected to the aptamer using a 5′-hexylamine linker phosphoramidite.

In a further aspect, TFPI aptamers are incorporated into pharmaceutical compositions for use in accordance with any of the methods described herein. The pharmaceutical compositions will generally include a therapeutically effective amount of the active component(s) of the therapy, e.g., a TFPI aptamer of the invention that is dissolved or dispersed in a pharmaceutically acceptable carrier or medium. Examples of pharmaceutically acceptable carriers include, but are not limited to, physiological saline solution, phosphate buffered saline solution, and glucose solution. However it is contemplated that other pharmaceutically acceptable carriers may also be used. Examples of other pharmaceutically acceptable media or carriers include any and all solvents, dispersion media, coatings, antibacterial agents, antifungal agents, isotonic and absorption delaying agents and the like. Pharmaceutically acceptable carriers that may be used in the compositions include, but are not limited to, ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The use of such media and agents for pharmaceutically active substances is well known in the art.

The pharmaceutical compositions may also contain pharmaceutically acceptable excipients, such as preserving, stabilizing, wetting or emulsifying agents, solution promoters, salts, or buffers for modifying or maintaining pH, osmolarity, viscosity, clarity, color, sterility, stability, rate of dissolution or absorption of the formulation. For solid compositions, excipients include pharmaceutical grades of mannitol, lactose, starch, magnesium stearate, sodium saccharin, talcum, cellulose, glucose, sucrose, magnesium carbonate and the like.

The pharmaceutical compositions are prepared according to conventional mixing, granulating or coating methods, and typically contain 0.1% to 99.9%, for example, 0.1% to 75%, 0.1% to 50%, 0.1% to 25%, 0.1% to 10%, 0.1 to 5%, preferably 1% to 50%, of the active component.

The formulation of pharmaceutical compositions is known to one of skill in the art. Typically, such compositions may be formulated as injectables, either as liquid solutions or suspensions; solid forms suitable for solution in, or suspension in, liquid prior to injection; as tablets or other solids for oral administration; as time release capsules for slow release formulations; or in any other form currently used, including eye drops, creams, lotions, salves, inhalants and the like. The compositions may also be formulated as suppositories, using for example, polyalkylene glycols as the carrier. In some embodiments, suppositories are prepared from fatty emulsions or suspensions. The use of sterile formulations, such as saline-based washes, by surgeons, physicians or health care workers to treat a particular area in the operating field may also be particularly useful.

The compositions may be formulated as oral dosage forms, such as tablets, capsules, pills, powders, granules, elixirs, tinctures, suspensions, syrups and emulsions. For instance, for oral administration in the form of a tablet or capsule (e.g., a gelatin capsule), the active drug component can be combined with an oral, non-toxic, pharmaceutically acceptable carrier, such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars, such as glucose or beta-lactose, corn sweeteners, natural and synthetic gums, such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethylene glycol and the like. Disintegrators include, without limitation, starch, methyl cellulose, agar, bentonite, xanthan gum starches, agar, alginic acid or its sodium salt, or effervescent mixtures and the like. Diluents, include, e.g., lactose, dextrose, sucrose, mannitol, sorbitol, cellulose and/or glycine.

Pharmaceutical compositions can also be formulated in liposome delivery systems, such as small unilamellar vesicles, large unilamellar vesicles and multilamellar vesicles. Liposomes can be formed from a variety of phospholipids containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564, which is hereby incorporated by reference in its entirety for all purposes and in particular for all teachings related to liposome delivery systems. For example, the aptamers described herein can be provided as a complex with a lipophilic compound or non-immunogenic, high molecular weight compound constructed using methods known in the art. Additionally, liposomes may bear aptamers on their surface for targeting and carrying cytotoxic agents internally to mediate cell killing. An example of nucleic acid-associated complexes is provided in U.S. Pat. No. 6,011,020, which is hereby incorporated by reference in its entirety for all purposes and in particular for all teachings related to nucleic acid-associated complexes.

The compositions of the invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compositions of the invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

The compositions of the invention may also be used in conjunction with medical devices.

The quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

Pharmaceutical formulations can conveniently be presented in unit dosage form and can be prepared by any of the methods well known in the art of pharmacy. All methods include the step of bringing into association a compound or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation. Such formulations are well known to those skilled in the art, and general methods for preparing them are found in any standard pharmacy school textbook, for example, Remington: The Science and Practice of Pharmacy, A. R. Gennaro, ed. (1995), the entire disclosure of which is incorporated herein by reference for all purposes and in particular for all teachings related to the preparation of pharmaceutical formulations.

Methods of Treatment

In one aspect, the present invention provides methods of treatment comprising modulating TFPI protein concentration and/or TFPI protein function.

In one aspect, the present invention provides methods of treatment that modulate TFPI protein concentration. In one aspect, the present invention provides methods for increasing the plasma concentration of TFPI protein in a subject by administering a TFPI modulator to the subject. In a further aspect, the methods include administering an aptamer to the subject. In an exemplary embodiment, the aptamer is BAX499 (SEQ ID NO: 1). In certain embodiments, the administration of a TFPI modulator such as BAX499 increases TFPI protein plasma levels without increasing TFPI mRNA levels. In yet further embodiments, BAX499 increases TFPI protein plasma levels by delaying proteolytic degradation of TFPI (see FIG. 14).

In further embodiments, the present invention provides methods of treatment that modulate TFPI protein clearance. In one aspect, the present invention provides methods for reducing TFPI clearance in a subject by administering a TFPI modulator to the subject. In a further aspect, the methods include administering an aptamer to the subject. In an exemplary embodiment, the aptamer is BAX499 (SEQ ID NO: 1). In still further embodiments, BAX499 interferes with TFPI clearance by disrupting TFPI binding to the receptor LRP-1 (for example, see FIG. 12).

Although for the sake of clarity much of the discussion herein focuses on treatment with the aptamer BAX499 and its use as a modulator of TFPI, it will be appreciated that other modulators of TFPI function and/or protein plasma levels can be utilized in accordance with the present invention. Additional examples of modulators of use in accordance with the methods described herein include without limitation peptides, antibodies, and small molecules. Small molecules of use in the invention can include without limitation fucoidan or sulfated or sulfonated polysaccharides or peptides, such as those described in U.S. Ser. No. 61/592,554, filed Jan. 30, 2012 and U.S. Ser. No. 61/592,549, filed on Jan. 30, 2012, each of which is hereby incorporated by reference in its entirety for all purposes, and in particular for all teachings related to small molecules and compositions for use in the treatment of bleeding disorders.

The efficacy and/or progress of treatment with a modulator of TFPI in accordance with any of the above can be evaluated using assays of either TFPI protein concentrations/levels or of TFPI clearance. For example, TFPI protein concentration can be determined by measuring the amount of TFPI in a sample. This is typically determined from a sample of biological fluid, such as blood, peritoneal fluid, or cerebrospinal fluid. TFPI can be cultured from the biological fluid in a manner suitable for growth or identification of surviving TFPI. TFPI clearance can be measured by determining TFPI protein concentration from samples of fluid taken over a period of time after treatment. Sampling time points can be 0.5, 1, 2, 3, 5, 8, 10, 20, and 35 minutes. Further data may be obtained by measuring over a period of time, preferably a period of days, blood coagulation.

Determining TFPI protein concentration or clearance can be accomplished using assays known in the art. Such assays can include immunological assays. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. (See, e.g., Self et al., Curr. Opin. Biotechnol 7:60-65 (1996)). The term immunoassay encompasses techniques including, without limitation, enzyme immunoassays (EIA) such as enzyme multiplied immunoassay technique (EMIT); enzyme-linked immunosorbent assay (ELISA); IgM antibody capture ELISA (MAC ELISA); and microparticle enzyme immunoassay (MEIA); immunohistochemical assay; capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. (See, e.g., Schmalzing et al., Electrophoresis, 25 18:2184-93 (1997); Bao, J. Chromatogr. B. Biomed. Sci., 699:463-80 (1997)). Liposome immunoassays, such as flow-injection liposome immunoassays and liposome immunosensors, are also suitable for use in the present invention. (See, e.g., Rongen et al., J. Immunol. Methods, 204:105-133 (1997)). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)). These assays are hereby incorporated by reference in their entirety for all purposes and in particular for all teachings related to measuring TFPI protein concentration.

When using ELISA to measure TFPI protein concentration or clearance, EDTA can be added to samples and standards for disrupting the interaction between TFPI and BAX499. For total TFPI quantification, wells of a microtiter plate (Nunc Maxisorp) can be coated with 1 μg/mL of a monoclonal anti human KD2 specific TFPI antibody (Sanquin, White label; MW1845). The samples can be incubated with a polyclonal rabbit anti hTFPI antibody (ADG72; American Diagnostica), washed with TBST and incubated with a goat anti rabbit HRP labeled antibody (A0545; Sigma). Color can be developed by addition of 100 μL of substrate (SureBlue TMP, KLP) and absorbance measured with a microtiter plate reader (Thermo Appliskan Reader). The fl-TFPI ELISA can be performed using a monoclonal anti-human C-terminus specific TFPI antibody (Sanquin, White label; MW1848) as a capture antibody. Purified endogenous fl-TFPI, expressed by SKHep cells, can be used as standard protein for quantification (Baxter Innovations GmbH).

Western blot (immunoblot) analysis can be used to detect and quantify TFPI concentration for determining TFPI concentration or clearance. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the antigen. The anti-antigen antibodies specifically bind to the antigen on the solid support. These antibodies can be directly labeled or alternatively can be subsequently detected using labeled antibodies (e.g., labeled sheep anti-mouse antibodies) that specifically bind to the anti-antigen antibodies.

Suitable TFPI antibodies that can be used for immunological assays that detect TFPI protein concentration for determining TFPI concentration or clearance can be a mouse monoclonal antibody against human TFPI Kunitz 2; clone M105272 (Fitzgerald Industries International, cat #10R-T142A; total TFPI), or a mouse monoclonal antibody against human TFPI C-terminal domain, clone M105274 (Fitzgerald Industries International, cat #10R-T144A; full length TFPI). A suitable primary detection antibody can be a rabbit polyclonal antibody against human TFPI (American Diagnostica Inc, cat #ADG72). Suitable TFPI can be full-length, recombinant human full-length TFPI (Baxter, lot N2268/1 0102).

A detectable moiety can be used to detect TFPI protein concentration for determining TFPI concentration or clearance. A variety of detectable moieties are well known to those skilled in the art, and can be any material detectable by spectroscopic, photochemical, biochemical, immunochemical, electrical, optical or chemical means. Detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, metals, and the like. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for nucleic acids or proteins is suitable for sensitive, non-radioactive detection of nucleic acids or protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. A urease detection system can be used with a substrate such as urebromocresol purple (Sigma Immunochemicals; St. Louis, Mo.). Other proteins capable of specifically binding immunoglobulin constant regions, such as protein A or protein G can also be used as a label agent. These proteins exhibit a strong non-immunogenic reactivity with immunoglobulin constant regions from a variety of species as described in the art (see, e.g., Kronval et al., J. Immunol. 111:1401-1406 (1973); Akerstrom et al., J. Immunol. 135:2589-2542 (1985), which are hereby incorporated by reference in their entirety for all purposes and in particular for all teachings related to measuring TFPI protein concentration.

In one aspect, the present invention provides methods of treatment that modulate TFPI function. TFPI protein function can be assessed using methods known in the art and described herein, including without limitation FX Activation assays, plasma-based thrombin generation assays, rotation thromboelastometry (ROTEM®), whole blood clotting assays, thromboelastography (TEG), and calibrated automated thrombogram (CAT) assays.

In a further aspect, the present invention provides methods and compositions for inhibiting TFPI protein function, particularly inhibiting TFPI's effects as an anticoagulant. In certain embodiments, BAX499 is administered to subjects in an amount effective to inhibit TFPI's anticoagulant activity. In still further embodiments, BAX499 is administered in a dose ranging from about 1 mg to about 100 mg. In further embodiments, BAX499 is administered in a dose ranging from about 1.1-10, 1.2-9.5, 1.3-9, 1.4-8.5, 1.5-8, 1.6-7.5, 1.7-7, 1.8-6.5, 1.9-6, 2-5.5, 2.1-5, 2.2-4.5, 2.3-4, 2.5-3 mg. In yet further embodiments, the BAX499 is administered in a dose ranging from about 1 nM to about 2000 nM. In further embodiments, the BAX499 is administered in a dose ranging from about 1-5, 1.1-4.5, 1.2-4, 1.3-3.5, 1.4-3, 1.5-2.5, 1.6-2 nM.

As will be appreciated, effective dosage formulations are those containing an effective dose, or an appropriate fraction thereof, of the active ingredient, or a pharmaceutically acceptable salt thereof. A prophylactic or therapeutic dose typically varies with the nature and severity of the condition to be treated and the route of administration. The dosage, and perhaps the dosing frequency, will also vary according to the age, body weight and response of the individual patient. In some embodiments, for the compounds of the invention, the total dose in a unit dosage form of the invention ranges from about 1 mg to about 1000 mg, e.g., from about 2 mg to about 500 mg, e.g., from about 10 mg to about 200 mg, e.g., from about 20 mg to about 100 mg, e.g., from about 20 mg to about 80 mg, e.g., from about 20 mg to about 60 mg.

In a still further aspect, the present invention provides methods and compositions for modulating a homeostasis between concentrations of BAX499 and TFPI protein plasma levels in a subject. As discussed further in the Examples section herein, administering BAX499 can inhibit TFPI protein function. Although lower concentrations of BAX499 effectively inhibit TFPI protein function, as increasing amounts of BAX499 are administered to a subject and/or build up in the plasma, there is a concomitant increase in TFPI protein plasma levels in the subject. This increase in TFPI protein plasma levels seems to be, without being limited by theory, due to release of TFPI protein from intracellular stores rather than release of membrane-bound TFPI (see FIGS. 10-11). BAX499 inhibition of TFPI function was more effective in the patient population studied when TFPI protein plasma levels were close to physiological levels (e.g., around 1.3 nm—see FIG. see FIGS. 18-19) than at higher TFPI protein plasma levels (e.g., 5 nm or higher, see FIGS. 18-20).

As shown in FIG. 20, the concentration of BAX499 needed to inhibit TFPI's anticoagulant effect increases exponentially: an approximately 50-fold excess of BAX 499 neutralizes 0.2 nM TFPI added to FVIII-inhibited plasma whereas a 140-fold excess is required for neutralization of 7.3 nM additional fl-TFPI. Thus, a pair of BAX 499 and TFPI concentrations which would be under the fitted line (FIG. 20) would support procoagulant activity of BAX 499, whereas a pair above the fitted line would result in a net anticoagulant effect. As will be appreciated, fine tuning the concentrations of BAX499 and TFPI protein in this way allows control of the use of BAX499 as a procoagulant to treat bleeding disorders or as an anticoagulant to treat clotting disorders.

In a still further aspect, the present invention provides a method of determining a pharmaceutically effective dose of a TFPI modulator for promoting coagulation in a subject. In some embodiments, the method includes measuring clotting time, clot size, and clot stability using methods known in the art from subjects provided varying doses of the TFPI modulator. Such dose response analyses provide a way to identify the optimal concentration of the TFPI modulator. In exemplary embodiments, the TFPI modulator is an aptamer. In further embodiments, the modulator is BAX499.

In a further aspect, administering BAX499 affects and, in some embodiments, complements, the procoagulant effects of co-administered FVIII. The FVIII may be administered prior to, subsequent to, or simultaneously with, BAX499. In further embodiments, the administration of BAX499 together with FVIII reduces the amount of FVIII needed to increase coagulation—in other words, BAX499 together with FVIII has the equivalent effect on coagulation as a higher concentration of FVIII (see Table 11 in Examples section below). In further embodiments, BAX499 increases the procoagulant effect of FVIII by from about 1.5 fold to about 10 fold higher as compared to when FVIII is administered alone. In yet further embodiments, BAX499 increases the procoagulant effect of FVIII by about 2, 2.5, 3, 3.5, 4, 4.5, 5, 5.5, 6, 6.5, 7.5, 8, 8.5, 9, and 9.5 fold over the increase in coagulation seen upon administering FVIII alone.

Administering TFPI modulators such as BAX499 can in further aspects be used to treat blood disorders such as bleeding disorders and clotting disorders. The procoagulant versus anticoagulant effect of BAX499 can be fine-tuned based on the concentration of plasma TFPI protein levels, which can be measured as described herein.

In further embodiments, the subjects receiving BAX499 in accordance with any of the methods described herein suffer from a bleeding disorder such as Hemophilia A.

In further embodiments, TFPI modulators described herein, including BAX499, are provided for use as a medicament. In further embodiments, BAX499 is provided for use as a medicament at any of the dosages described herein, including without limitation doses ranging from about 1.1-10, 1.2-9.5, 1.3-9, 1.4-8.5, 1.5-8, 1.6-7.5, 1.7-7, 1.8-6.5, 1.9-6, 2-5.5, 2.1-5, 2.2-4.5, 2.3-4, 2.5-3 mg, or in doses ranging from about 1-5, 1.1-4.5, 1.2-4, 1.3-3.5, 1.4-3, 1.5-2.5, 1.6-2 nM. In still further embodiments, BAX499 is provided in a unit dosage form ranging from about 1 mg to about 1000 mg, e.g., from about 2 mg to about 500 mg, e.g., from about 10 mg to about 200 mg, e.g., from about 20 mg to about 100 mg, e.g., from about 20 mg to about 80 mg, e.g., from about 20 mg to about 60 mg.

In still further embodiments, the present invention provides methods for the use of a TFPI modulator, including without limitation BAX499, for treating blood disorders such as bleeding disorders and clotting disorders. In further embodiments, present invention provides methods for the use of a TFPI modulator, including without limitation BAX499, for treating Hemophilia A. In still further embodiments, the present invention provides methods for the use of BAX499 for treating blood disorders, including bleeding disorders, clotting disorders, and Hemophilia A, where BAX499 is provided at any of the dosages described herein, including without limitation doses ranging from about 1.1-10, 1.2-9.5, 1.3-9, 1.4-8.5, 1.5-8, 1.6-7.5, 1.7-7, 1.8-6.5, 1.9-6, 2-5.5, 2.1-5, 2.2-4.5, 2.3-4, 2.5-3 mg, or in doses ranging from about 1-5, 1.1-4.5, 1.2-4, 1.3-3.5, 1.4-3, 1.5-2.5, 1.6-2 nM. In still further embodiments, BAX499 is provided in a unit dosage form ranging from about 1 mg to about 1000 mg, e.g., from about 2 mg to about 500 mg, e.g., from about 10 mg to about 200 mg, e.g., from about 20 mg to about 100 mg, e.g., from about 20 mg to about 80 mg, e.g., from about 20 mg to about 60 mg.

Methods of Identifying TFPI Modulators

In one aspect, the present invention provides methods for identifying additional modulators of TFPI. In an exemplary embodiment and as is discussed in further detail below, methods for identifying additional modulators of TFPI can include the use of BAX499, particularly in competitive assays.

Examples of additional modulators that can be identified in accordance with the present invention include without limitation peptides, antibodies, and small molecules. Small molecules of use in the invention can include without limitation fucoidan or sulfated or sulfonated polysaccharides or peptides, such as those described in U.S. Ser. No. 61/592,554, filed Jan. 30, 2012 and U.S. Ser. No. 61/592,549, filed on Jan. 30, 2012, each of which is hereby incorporated by reference in its entirety for all purposes, and in particular for all teachings related to modulators of TFPI.

Modulators of TFPI can inhibit or activate TFPI protein function, increase or decrease intracellular or membrane bound TFPI plasma protein levels, or increase or decrease TFPI clearance. Methods of determining TFPI concentration and TFPI clearance are described in detail herein with respect to methods of treatment, and those same assays can also be used to identify a molecule that inhibits or activates TFPI protein function, increases or decreases intracellular or membrane bound TFPI plasma protein levels, or increases or decreases TFPI clearance.

Identification of additional modulators of TFPI protein function can be accomplished using methods known in the art and described herein, including without limitation, a competitive assay between a molecule and BAX499, FX Activation assays, plasma-based thrombin generation assays, rotation thromboelastometry (ROTEM®), whole blood clotting assays, thromboelastography (TEG), and calibrated automated thrombogram (CAT) assays.

Assays that identify additional modulators of TFPI can utilize naturally occurring or recombinant TFPI. Suitable TFPI can be human fl-TFPI (Baxter Innovations GmbH). Suitable levels of TFPI can be 775 nM injected at 5 mL/kg, i.v. TFPI can be administered by itself, complexed to a 10-fold molar excess of ARC17480, or complexed to a 10-fold molar excess of BAX 499. Suitable rodents can be 20-25 g C57B16 male mice.

In embodiments in which the competitive assay is a binding-competition assay, BAX499 can be used as a control. For example, a suitable assay may involve incubating 10 nM human TFPI (American Diagnostica, Stamford, Conn., catalog #4500PC) with trace amounts of radiolabeled BAX499 and 5000 nM, 16667 nM, 556 nM, 185 nM, 61.7 nM, 20.6 nM, 6.86 nM, 2.29 nM, 0.76 nM or 0.25 nM of unlabeled competitor. In exemplary embodiments, the competitor molecule is another aptamer. A control aptamer is included in each experiment. For each aptamer, the percentage of radiolabeled control aptamer bound at each competitor aptamer concentration is used for analysis. The percentage of radiolabeled control aptamer bound is plotted as a function of aptamer concentration and fitted to the equation y=(max/(1+x/IC₅₀))+int, where y=the percentage of radiolabeled control aptamer bound, x=the concentration of aptamer, max=the maximum radiolabeled control aptamer bound, and int=the y-intercept, to generate an IC₅₀ value for binding-competition. The IC₅₀ of each aptamer is compared to the IC₅₀ of the control aptamer evaluated in the same experiment. An aptamer having substantially the same ability to bind may include an aptamer having an IC₅₀ that is within one or two orders of magnitude of the IC₅₀ of the control aptamer, and/or an aptamer having an IC₅₀ that is not more than 5-fold greater than that of the control aptamer evaluated in the same experiment.

The ability of an aptamer to affect TFPI biological function and/or to modulate blood coagulation may be further assessed in a calibrated automated thrombogram (CAT) assay in which BAX499 is used as a control aptamer. For example, a suitable assay may involve evaluation in a CAT assay in pooled hemophilia A plasma at 500 nM, 167 nM, 55.6 nM, 18.5 nM, 6.17 nM and 2.08 nM aptamer concentration. A control aptamer is included in each experiment. For the molecule(s) being tested, endogenous thrombin potential (ETP) and peak thrombin values at each aptamer concentration are used for analysis. The ETP or peak thrombin value for hemophilia A plasma alone is subtracted from the corresponding value in the presence of aptamer for each molecule at each concentration. Then, the corrected ETP and peak values are plotted as a function of aptamer concentration and fitted to the equation y=(max/(1+IC₅₀/x))+int, where y=ETP or peak thrombin, x=concentration of aptamer, max=the maximum ETP or peak thrombin, and int=the y-intercept, to generate an IC₅₀ value for both the ETP and the peak thrombin. The IC₅₀ of each aptamer is compared to the IC₅₀ of the control aptamer that is evaluated in the same experiment. A test molecule having substantially the same ability to modulate a biological function and/or to modulate blood coagulation may include an aptamer having an IC₅₀ that is within one or two orders of magnitude of the IC₅₀ of the control aptamer, and/or an aptamer for which one or both of the ETP and peak thrombin IC₅₀ of that molecule are not more than 5-fold greater than that of the control aptamer evaluated in the same experiment.

The ability of a molecule to modulate a biological function and/or to modulate blood coagulation may also be assessed by evaluating inhibition of TFPI in a Factor Xa (FXa) activity assay in which BAX499 is used as a control aptamer. A suitable assay may involve measuring the ability of FXa to cleave a chromogenic substrate in the presence and absence of TFPI, with or without the addition of aptamer. For example, 2 nM human FXa is incubated with 8 nM human TFPI. Then, 500 μM chromogenic substrate and aptamers are added and FXa cleavage of the substrate is measured by absorbance at 405 nm (A₄₀₅) as a function of time. Aptamers are tested at 500 nM, 125 nM, 31.25 nM, 7.81 nM, 1.95 nM and 0.49 nM concentrations. A control aptamer is included in each experiment. For each aptamer concentration, the A₄₀₅ is plotted as a function of time and the linear region of each curve is fitted to the equation y=mx+b, where y=A₄₀₅, x=the aptamer concentration, m=the rate of substrate cleavage, and b=the y-intercept, to generate a rate of FXa substrate cleavage. The rate of FXa substrate cleavage in the presence of TFPI and the absence of aptamer is subtracted from the corresponding value in the presence of both TFPI and aptamer for each molecule at each concentration. Then, the corrected rates are plotted as a function of aptamer concentration and fitted to the equation y=(Vₘₐₓ/(1+IC₅₀/x)), where y=the rate of substrate cleavage, x=concentration of aptamer, and Vₘₐₓ=the maximum rate of substrate cleavage, to generate an IC₅₀ and maximum (Vₘₐₓ) value. The IC₅₀ and Vₘₐₓ values of each aptamer are compared to the IC₅₀ and Vₘₐₓ values of the control aptamer evaluated in the same experiment. An aptamer having substantially the same ability to modulate a biological function and/or to modulate blood coagulation may include an aptamer having an IC₅₀ that is within one or two orders of magnitude of the IC₅₀ of the control aptamer, and/or an aptamer having an IC₅₀ that is not more than 5-fold greater than that of the control aptamer evaluated in the same experiment, and/or an aptamer having a V. value not less than 80% of the V. value of the control aptamer evaluated in the same experiment.

The present invention will now be further illustrated in the following examples, without being limited thereto.

### EXAMPLES

### Example 1

### BAX 499 Had Pro-Coagulant Effect in Non-Human Primate Model

This example describes the evaluation of tissue factor pathway inhibitor (TFPI) concentrations, both total and full-length TFPI, in plasma samples from cynomolgus monkeys.

### EXPERIMENTAL PROCEDURES

### Sample Collection and Shipment

Plasma samples, anticoagulated in dipotassium (K2)EDTA, were collected from monkeys prior to dosing (Week −1), on Day 1 at 2, 8, 24, and 48 hours post dose, on Days 10, 87, and 178 at predose and 2, 8, 24, 48, and 72 hours post dose, and on Day 181 at 120 and 240 hours post dose. In addition, samples from some monkeys were collected on Day 239 following an 8-week recovery period after the last BAX 499 dose on Day 181. Plasma samples were stored at −80° C.

TFPI concentrations were analyzed in plasma samples from 4 animals (2 males and 2 females) in each of 4 dose groups. Group 1 monkeys (1006-M, 1105-M, 1505-F, and 1506-F) received vehicle control. Group 2 monkeys (2005-M, 2006-M, 2505-F, and 2506-F) received 1.25 mg/kg BAX 499. Group 3 monkeys (3005-M, 3106-M, 3505-F, and 3506-F) received 3.75 mg/kg BAX 499. Group 4 monkeys (4005-M, 4006-M, 4505-F, and 4506-F) received 12.5 mg/kg BAX 499. Plasma samples from Week −1 were not available for two of the 16 monkeys evaluated and a Day 239 sample was available for all 16 monkeys evaluated.

### ELISA for Total and Full-Length Plasma TFPI

Each plasma sample was analyzed in two different ELISAs, one that measures the concentration of total plasma TFPI (referred to as “total TFPI”) and one that measures the concentration of full-length plasma TFPI. The general procedure for the two ELISAs was the same with the exception of the capture antibody used in the first step, and the extent of sample dilution required for measurement.

In each assay, a standard curve used to calculate values was generated using human TFPI due to the lack of availability of cynomolgus monkey TFPI. Results among monkeys were analyzed relative to one another, but not relative to absolute concentrations. The majority of the samples required dilution of 1000- to 3000-fold in order to fall within the range of the standard curve.

### Materials and Sample Preparation

### Supplies and Buffers

ELISA plate (Nunc Maxisorp, cat #442404)

EDTA, 0.5 M, pH 8.0 (USB, cat #15694)

Dry Milk (Bio-Rad, cat #170-6404)

TBS tablets (Amresco, cat #K859-200TABS)

Tween 20 (Bio-Rad, cat #170-6531

Sure Blue TMB Substrate solution (KPL, cat #52-00-02)

TBS (25 mM Tris-HCl, pH 7.4, 150 mM NaCl)

TBST (25 mM Tris-HCl, pH 7.4, 150 mM NaCl, 0.1% Tween 20)

Coating buffer (TBS)

Blocking buffer (TBS plus 2% (w/v) dry milk)

Washing buffer (1×TBS solution using TBS tablets, 0.1% (v/v) Tween 20)

Sample buffer (20 mM EDTA and 1% (w/v) dry milk in TBST)

Stopping reagent (1M HCl)

### Antibodies and Proteins

Capture antibody for total TFPI ELISA: mouse monoclonal antibody against human TFPI Kunitz 2; clone M105272 (Fitzgerald Industries International, cat #10R-T142A)

Capture antibody for full-length TFPI ELISA: mouse monoclonal antibody against human TFPI C-terminal domain, clone M105274 (Fitzgerald Industries International, cat #10R-T144A)

Primary detection antibody: rabbit polyclonal antibody against human TFPI (American Diagnostica Inc, cat #ADG72)

Secondary detection antibody: goat anti-rabbit IgG-horseradish peroxidase conjugated (Cell Signaling, cat #7074)

TFPI, full-length: recombinant human full-length TFPI used for standard curve and as internal control (Baxter, lot N2268/1 0102)

TFPI, Histidine (His)-tagged: recombinant TFPI containing amino acids 29-282, with a C-terminal His-tag used as internal control (R&D Systems, cat #2974-PI-010)

### Sample Preparation

Both internal controls, (TFPI, full-length and TFPI, His-tagged) were diluted to concentrations of 2.5 ng/mL and stored in one-time-use aliquots at −80° C. Both controls were used with each ELISA. The aliquot was thawed at 37° C. for 1 min, vortexed, centrifuged briefly and kept on ice until use.

TFPI for standards was thawed at 37° C. for 30 sec, vortexed, and centrifuged briefly. Dilutions were made in sample buffer to achieve 8, 6, 4, 2, 1, 0.5, 0.25 and 0.1 ng/mL, and kept on ice until use.

Pooled normal plasma (George King Bio-Medical, cat #1001) was used as an additional control. Plasma was thawed for 4 min at 37° C., centrifuged briefly and diluted 1:10 or 1:25 in sample buffer for the full-length and total TFPI ELISAs, respectively

Samples from the 6-month toxicity study were thawed at 37° C. and diluted with sample buffer (according to Table 1) such that the expected concentration of TFPI was within the standard range (0.25 to 8.0 ng/mL; 0.006 to 0.186 nM TFPI). A list of the upper and lower limits of quantification (ULOQ and LLOQ, respectively) can be found in Table 2 in Upper and Lower Limits of Quantification. These limits are dependent on the sample dilution.

TABLE 1
dilution level for plasma samples

| | | Group 1 | | Group 2 | | Group 3 | | Group 4 | |
|---|---|---|---|---|---|---|---|---|---|
| | | | Full- | | Full- | | Full- | | Full- |
| | | Total | length | Total | length | Total | length | Total | length |
| Week-1 | 0 hr | 1:25 | 1:10 | 1:25 | 1:10 | 1:25 | 1:10 | 1:25 | 1:10 |
| Day 1 | 2 hr | 1:25 | 1:10 | 1:100 | 1:25 | 1:100 | 1:100 | 1:100 | 1:100 |
| | 8 hr | 1:25 | 1:10 | 1:400 | 1:100 | 1:400 | 1:400 | 1:400 | 1:400 |
| | 24 hr | 1:25 | 1:10 | 1:1600 | 1:400 | 1:1600 | 1:1600 | 1:1600 | 1:1600 |
| | 48 hr | 1:25 | 1:10 | 1:1600 | 1:400 | 1:1600 | 1:1600 | 1:1600 | 1:1600 |
| Day 10 | 0 hr | 1:25 | 1:10 | 1:1600 | 1:400 | 1:1600 | 1:1600 | 1:1600 | 1:1600 |
| | 2 hr | 1:25 | 1:10 | 1:3000 | 1:1000 | 1:3000 | 1:3000 | 1:3000 | 1:3000 |
| | 8 hr | 1:25 | 1:10 | 1:3000 | 1:1000 | 1:3000 | 1:3000 | 1:3000 | 1:3000 |
| | 24 hr | 1:25 | 1:10 | 1:3000 | 1:1000 | 1:3000 | 1:3000 | 1:3000 | 1:3000 |
| | 48 hr | 1:25 | 1:10 | 1:3000 | 1:1000 | 1:3000 | 1:3000 | 1:3000 | 1:3000 |
| | 72 hr | 1:25 | 1:10 | 1:3000 | 1:1000 | 1:3000 | 1:3000 | 1:3000 | 1:3000 |
| Day 87 | 0 hr | 1:25 | 1:10 | 1:3000 | 1:1000 | 1:3000 | 1:3000 | 1:3000 | 1:3000 |
| | 2 hr | 1:25 | 1:10 | 1:3000 | 1:1000 | 1:3000 | 1:3000 | 1:3000 | 1:3000 |
| | 8 hr | 1:25 | 1:10 | 1:3000 | 1:1000 | 1:3000 | 1:3000 | 1:3000 | 1:3000 |
| | 24 hr | 1:25 | 1:10 | 1:3000 | 1:1000 | 1:3000 | 1:3000 | 1:3000 | 1:3000 |
| | 48 hr | 1:25 | 1:10 | 1:3000 | 1:1000 | 1:3000 | 1:3000 | 1:3000 | 1:3000 |
| | 72 hr | 1:25 | 1:10 | 1:3000 | 1:1000 | 1:3000 | 1:3000 | 1:3000 | 1:3000 |
| Day 178 | 0 hr | 1:25 | 1:10 | 1:3000 | 1:1000 | 1:3000 | 1:3000 | 1:3000 | 1:3000 |
| | 2 hr | 1:25 | 1:10 | 1:3000 | 1:1000 | 1:3000 | 1:3000 | 1:3000 | 1:3000 |
| | 8 hr | 1:25 | 1:10 | 1:3000 | 1:1000 | 1:3000 | 1:3000 | 1:3000 | 1:3000 |
| | 24 hr | 1:25 | 1:10 | 1:3000 | 1:1000 | 1:3000 | 1:3000 | 1:3000 | 1:3000 |
| | 48 hr | 1:25 | 1:10 | 1:3000 | 1:1000 | 1:3000 | 1:3000 | 1:3000 | 1:3000 |
| | 72 hr | 1:25 | 1:10 | 1:3000 | 1:1000 | 1:3000 | 1:3000 | 1:3000 | 1:3000 |
| Day 181 | 120 hr | 1:25 | 1:10 | 1:400 | 1:100 | 1:400 | 1:400 | 1:1600 | 1:1600 |
| | 240 hr | 1:25 | 1:10 | 1:100 | 1:25 | 1:100 | 1:100 | 1:400 | 1:400 |
| Day 239 | 0 hr | 1:25 | 1:10 | 1:25 | 1:10 | 1:25 | 1:10 | 1:25 | 1:10 |

TABLE 2
upper and lower limits of quantification for each sample dilution

| | LLOQ* | ULOQ** |
|---|---|---|
| Dilution | (nM) | (nM) |
| 1:10 | 0.06 | 1.86 |
| 1:25 | 0.15 | 4.65 |
| 1:100 | 0.58 | 18.61 |
| 1:400 | 2.33 | 74.42 |
| 1:1000 | 5.81 | 186.05 |
| 1:1600 | 9.30 | 297.68 |
| 1:3000 | 17.44 | 558.15 |

*LLOQ: lower limit of quantification;
**ULOQ: upper limit of quantification

### ELISA Procedure

The night before the ELISA, 100 μL of the appropriate capture antibody diluted in coating buffer to 1 μg/mL (M105272 for the total TFPI ELISA and M105274 for the full-length TFPI ELISA) was added to the wells of a 96-well ELISA plate, which was then covered and incubated overnight at 4° C. On the morning of the ELISA, the plate was brought to room temperature for 1 hour and washed 3 times with washing buffer. Freshly made blocking buffer (250 μL) was added to each well, and the plate was incubated for 1 hour at room temperature on a plate shaker. The plate was washed 3 times with washing buffer. Standard, control, or diluted sample (100 μL) was added to appropriate wells, and the plate was covered and incubated at room temperature for 2 hours on the plate shaker. The wells were washed 5 times in washing buffer, and 100 μL of the primary detection antibody (diluted to 0.5 μg/mL in sample buffer) was added to each well. The plate was covered and incubated at room temperature for 1 hour on the plate shaker. The wells were washed 5 times with washing buffer, and 100 μL of the secondary detection antibody was added to each well. For most plates, the secondary detection antibody was diluted 1:10,000 in sample buffer. For the plates measuring the samples from Group 1 monkeys, antibody was diluted 1:1000. The plate was incubated at room temperature on the plate shaker for 1 hour, protected from light. The wells were washed 5 times, and 100 μL TBM substrate solution (at room temperature) was added to each well. The plate was incubated at room temperature, on the plate shaker, protected from light, for 7 minutes (Group 1) or 8 minutes (Groups 2, 3, and 4). The reaction was stopped with the addition of 50 μL 1M HCl and mixed gently. Absorbance was read at 450 nm with a reference wavelength of 620 nm.

### Analysis of Results

Each sample was tested in duplicate. In order to determine the concentrations of TFPI in each sample, the reference OD was subtracted from the OD at 450 nm. The mean OD from the blank wells (no TFPI or sample) was subtracted from the OD reading of each well to obtain corrected OD values. A standard curve was then generated and fit using 4-parameter logistics with GraphPad Prism version 5.04. The mean of the corrected OD from each sample was fit to the standard curve and multiplied by the appropriate dilution factor in order to determine the concentration (ng/mL) of TFPI in each sample. This value was then converted to molar concentration using a molecular weight of 43,000 g/mol.

### Results

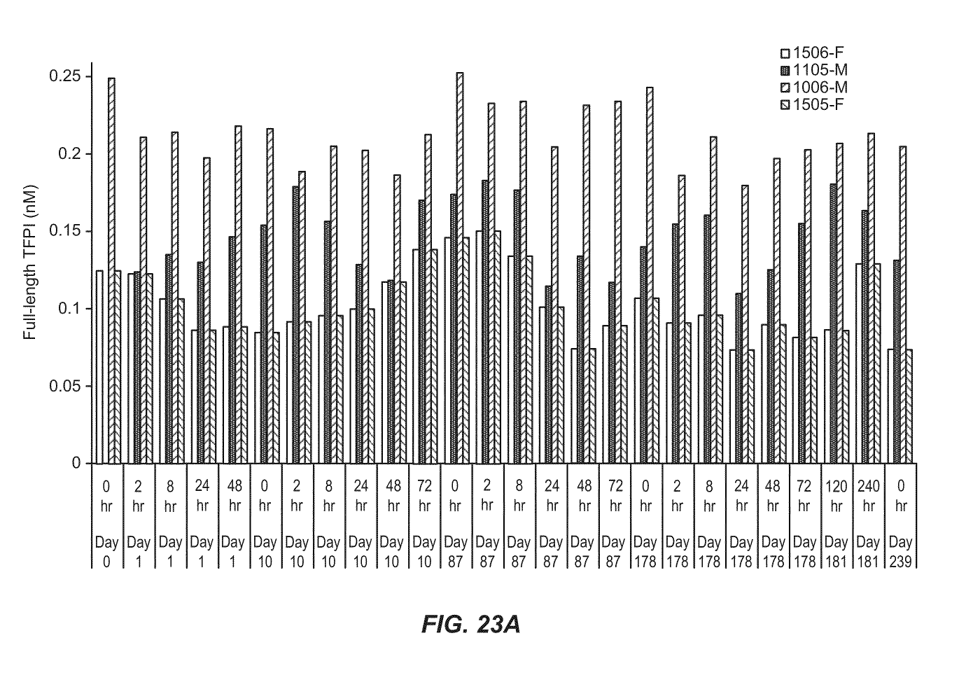

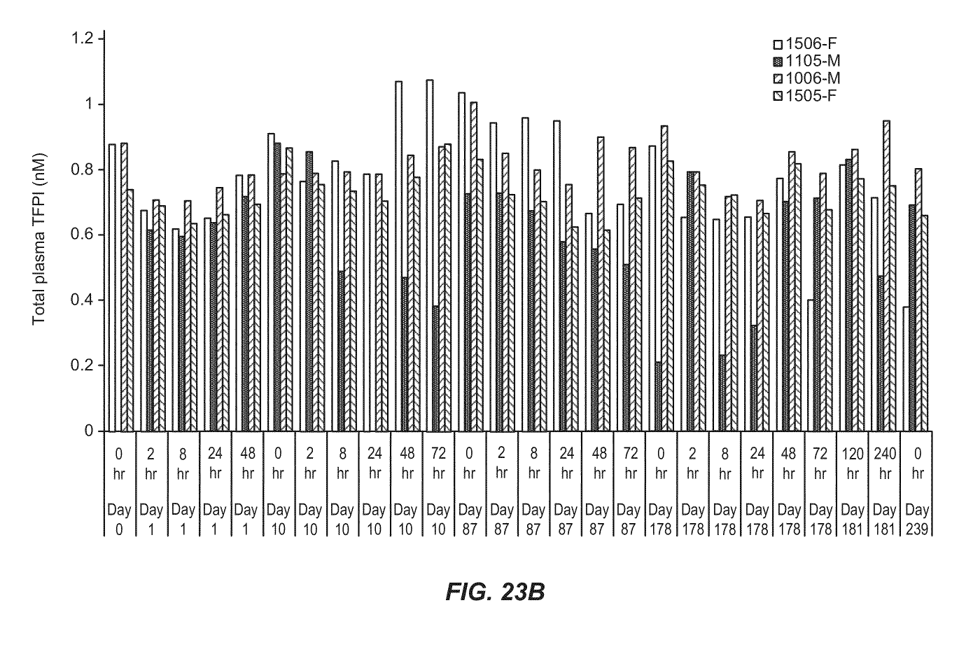

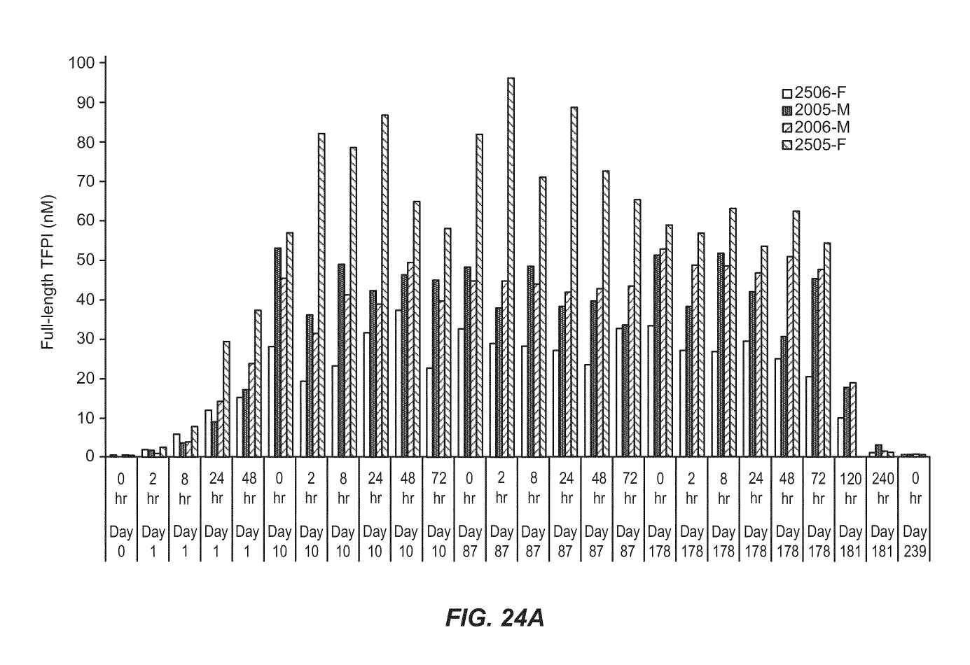

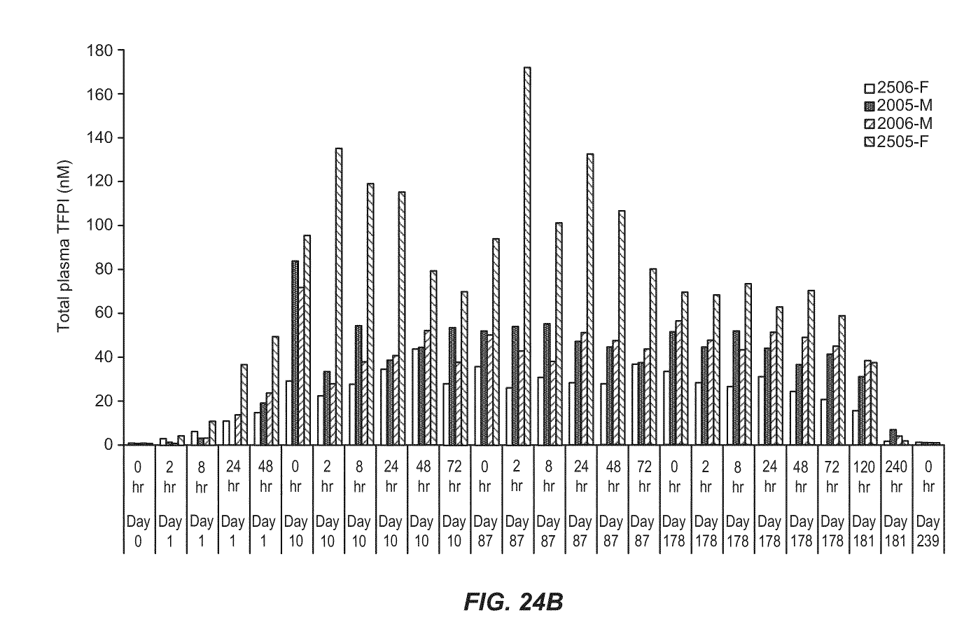

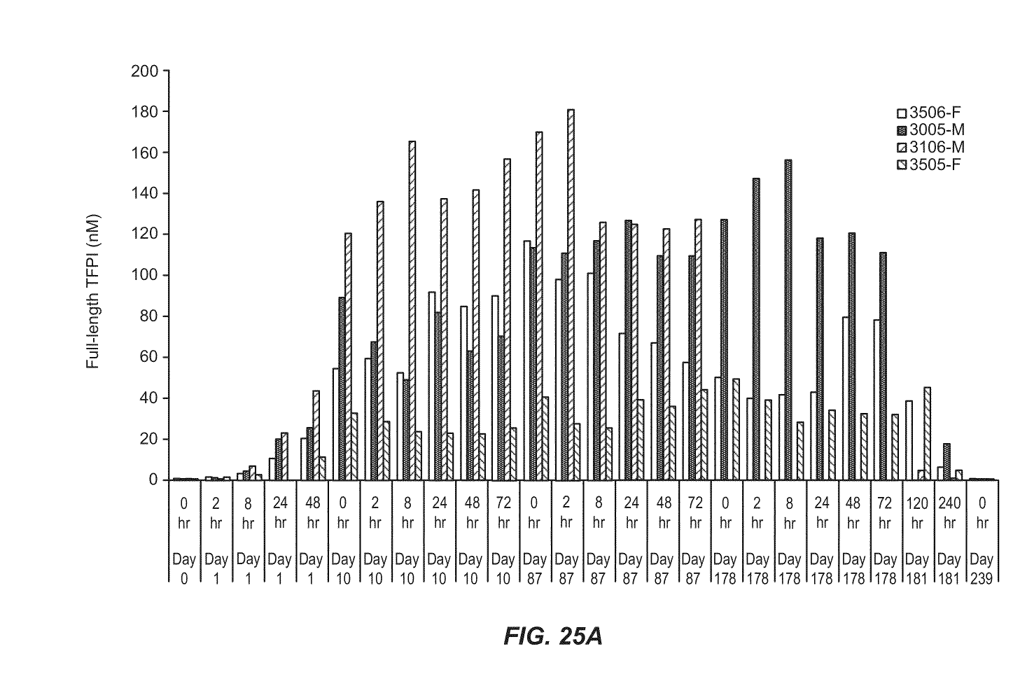

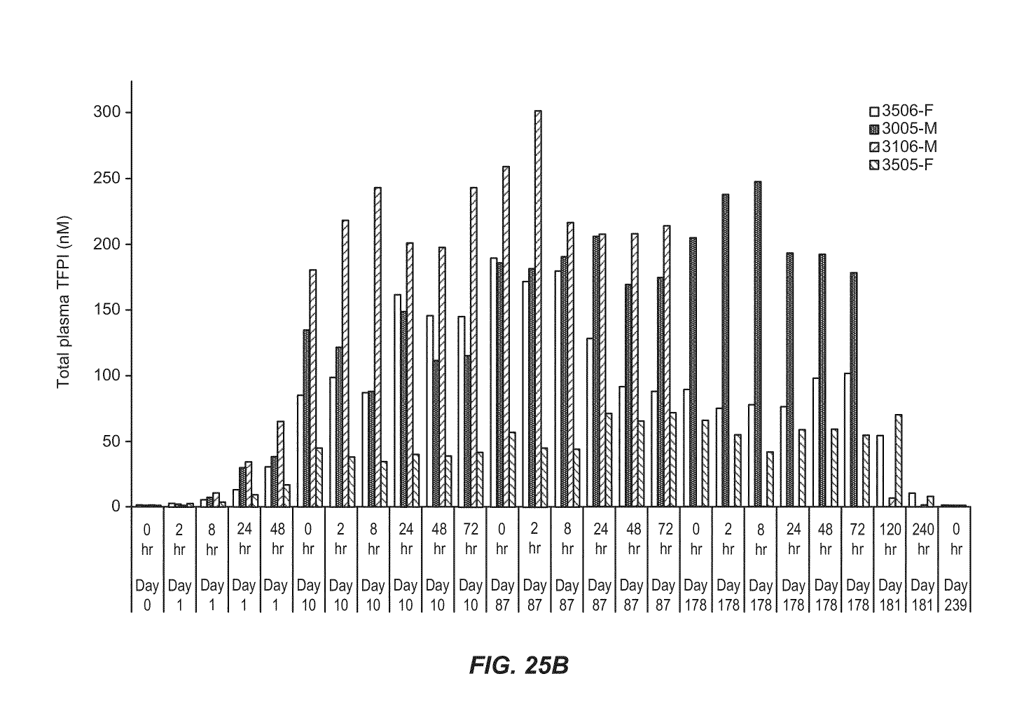

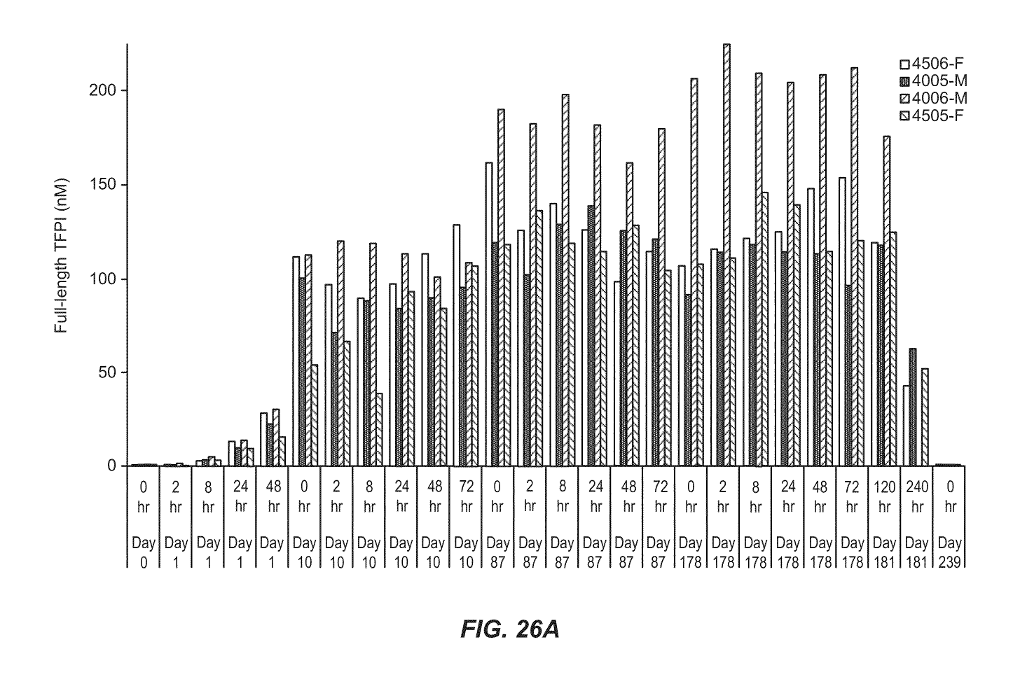

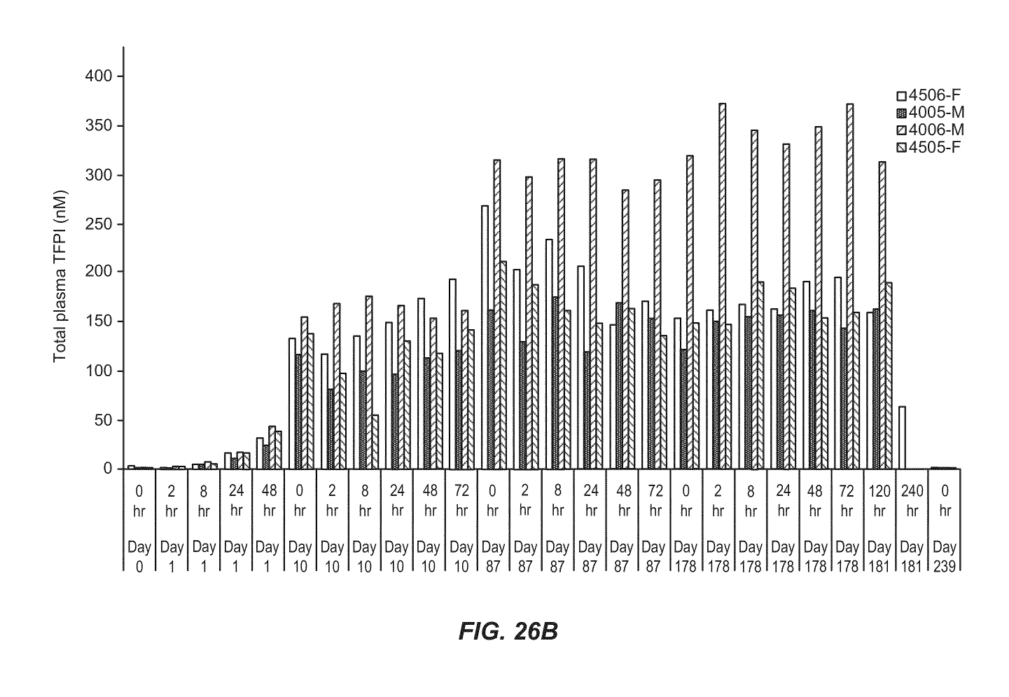

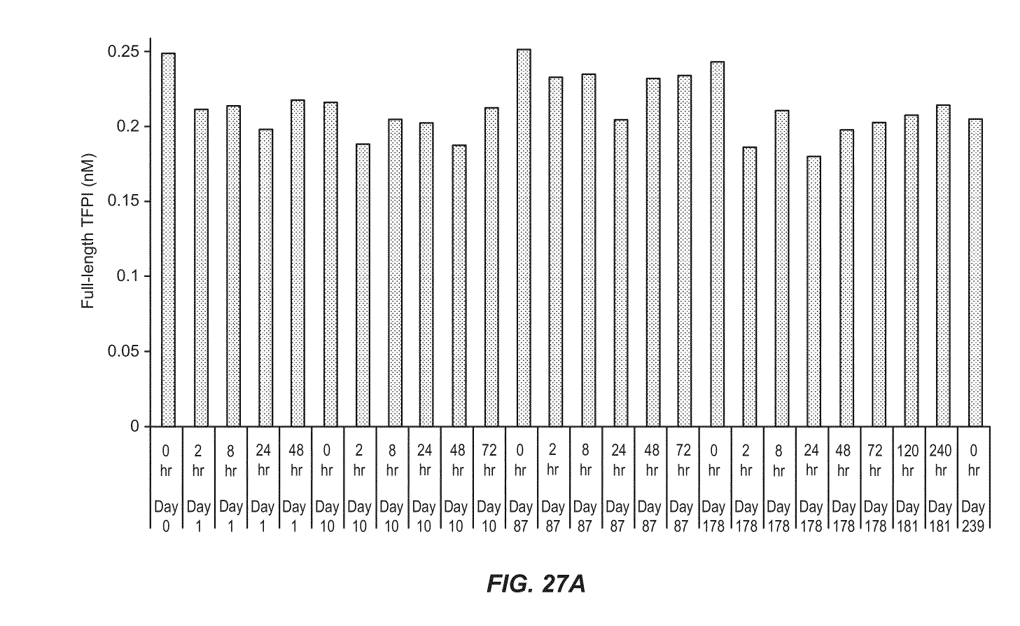

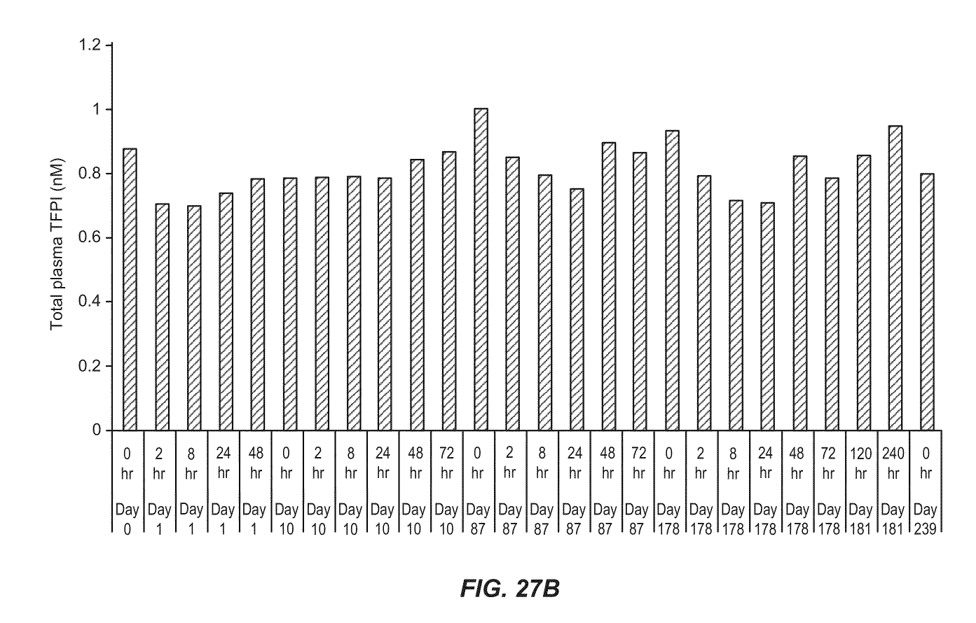

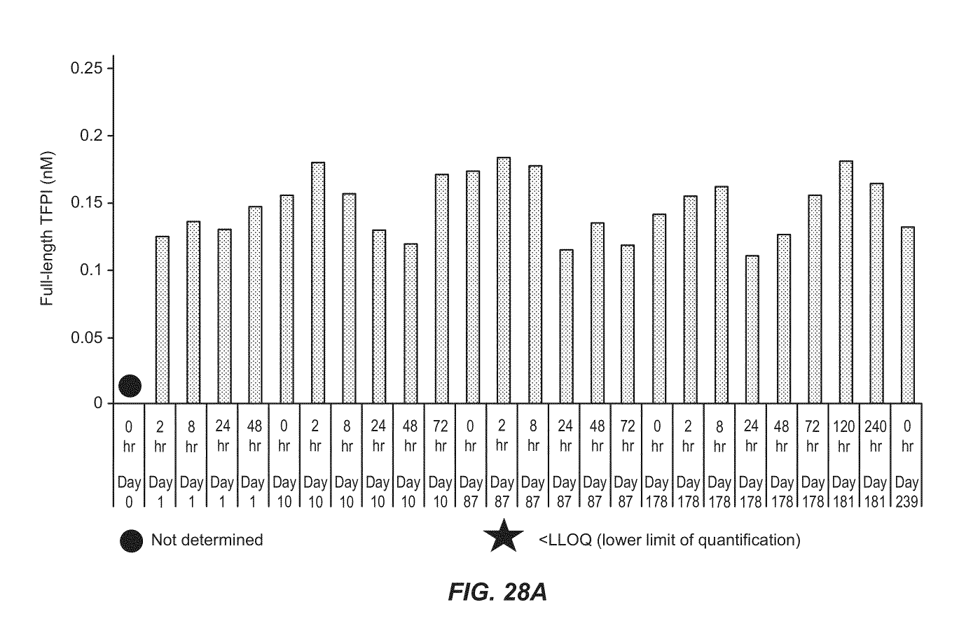

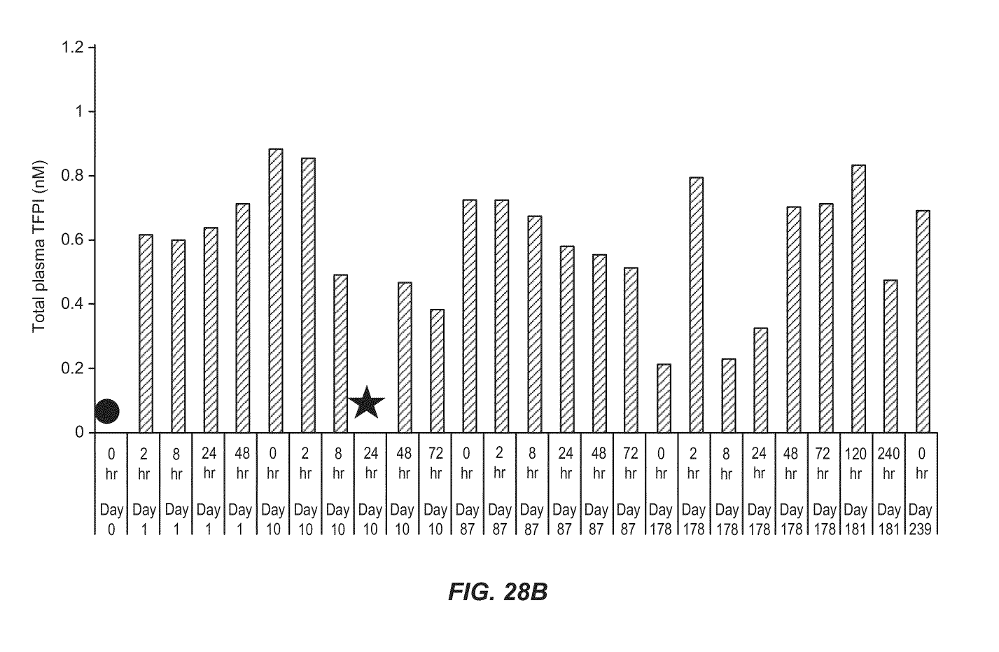

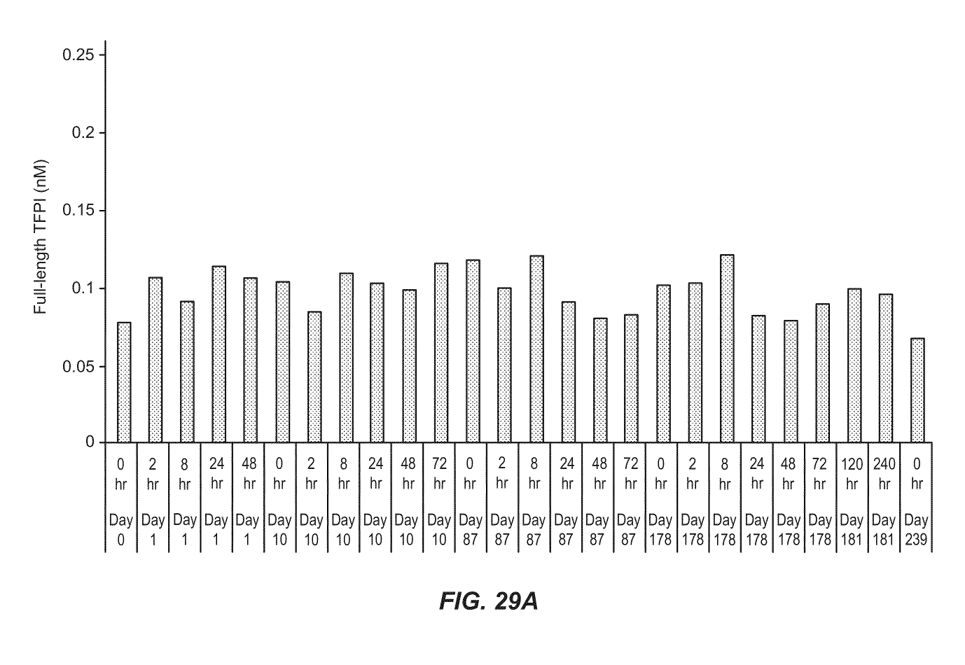

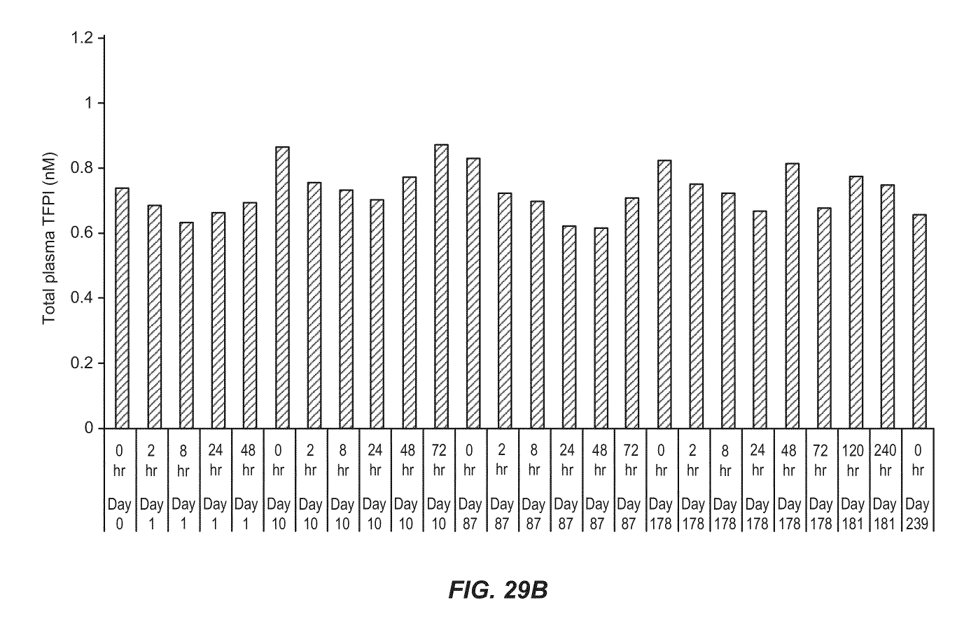

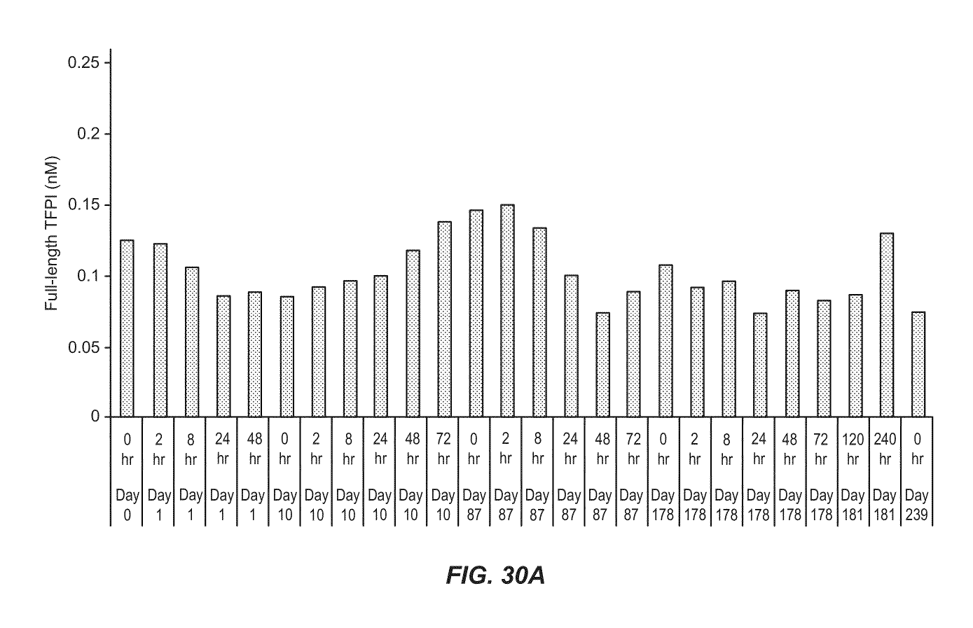

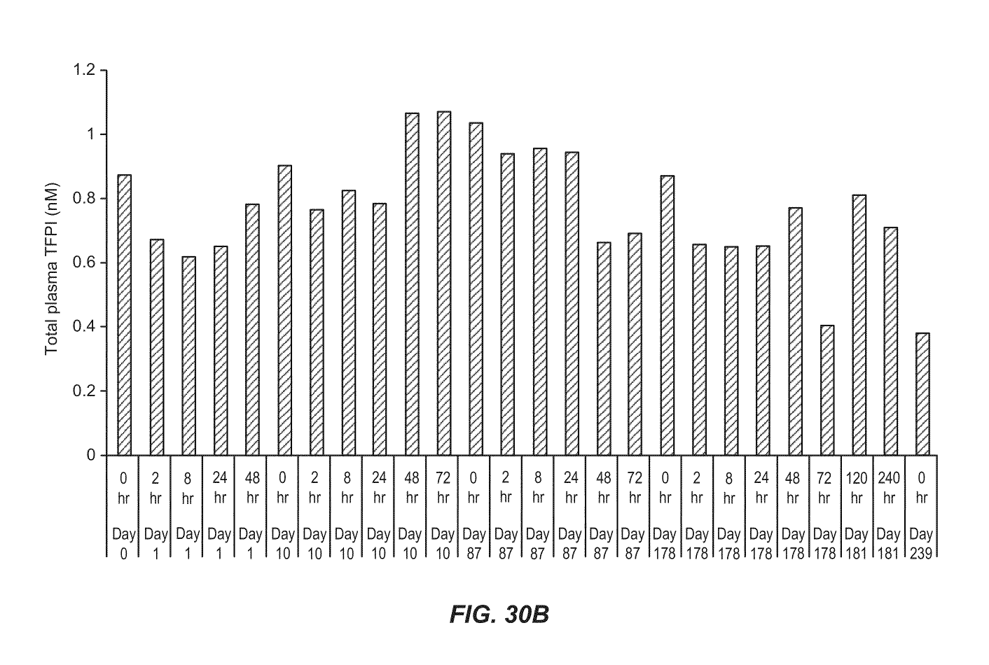

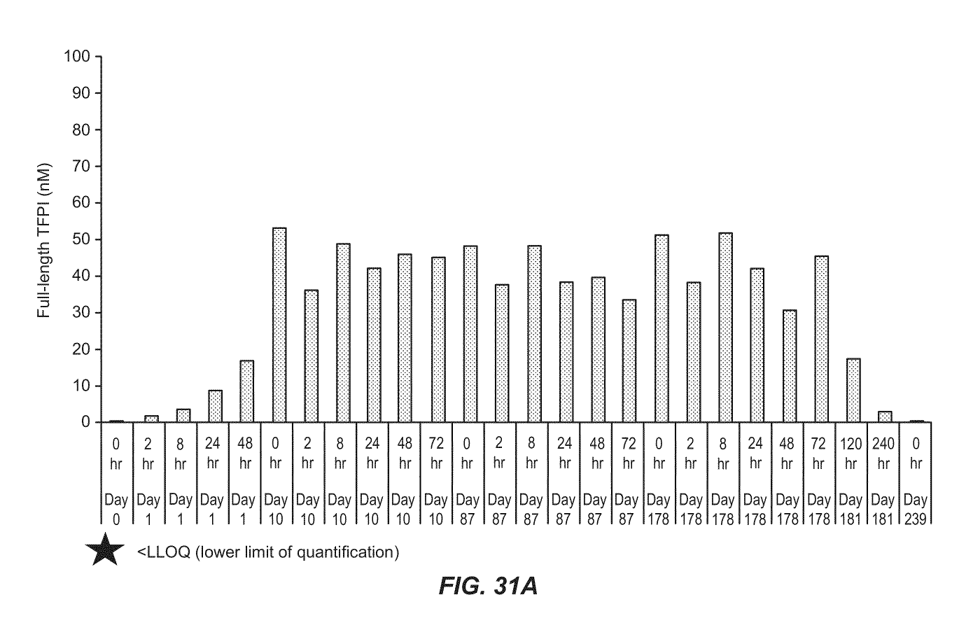

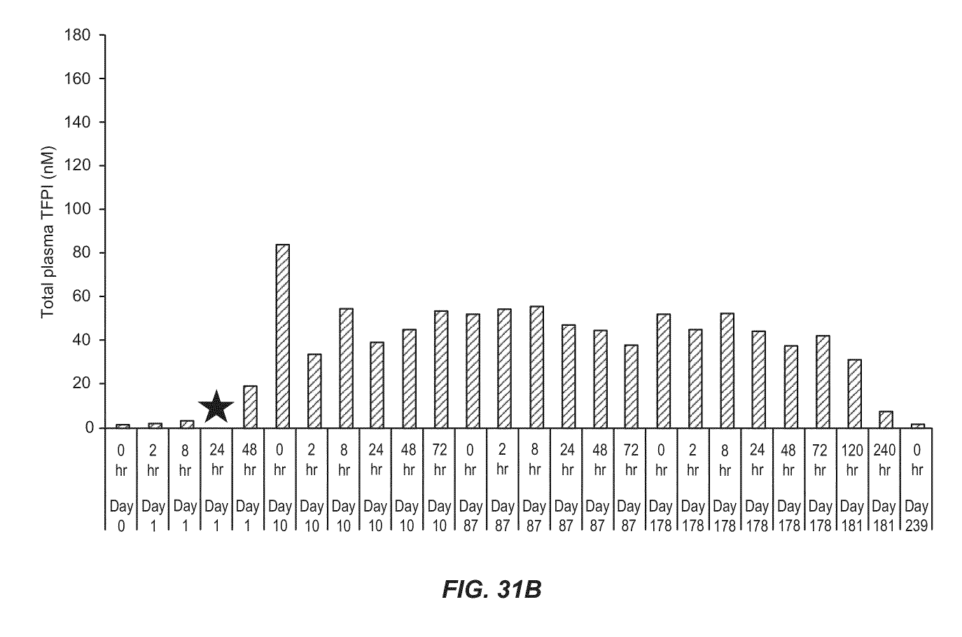

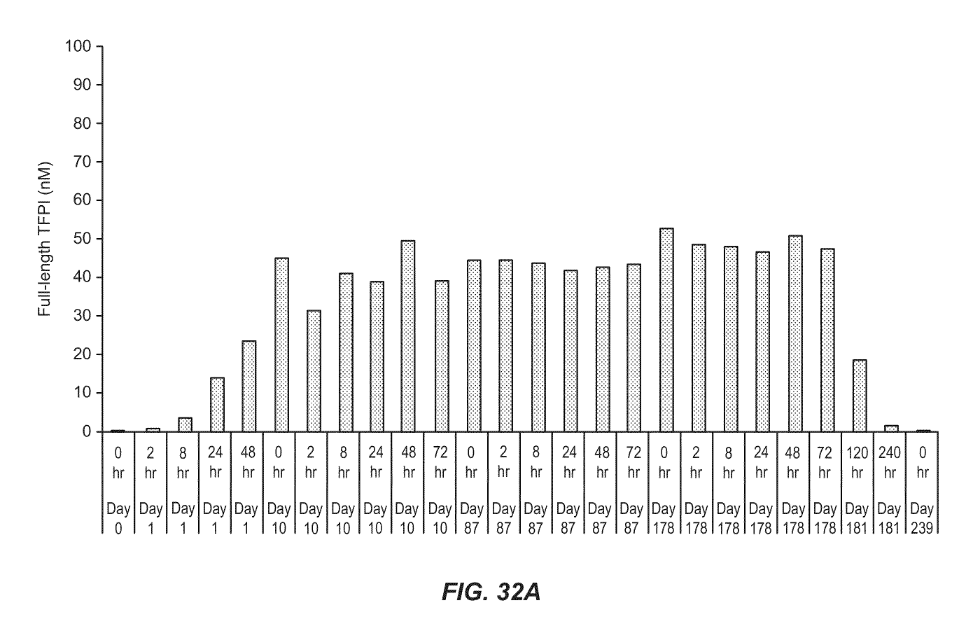

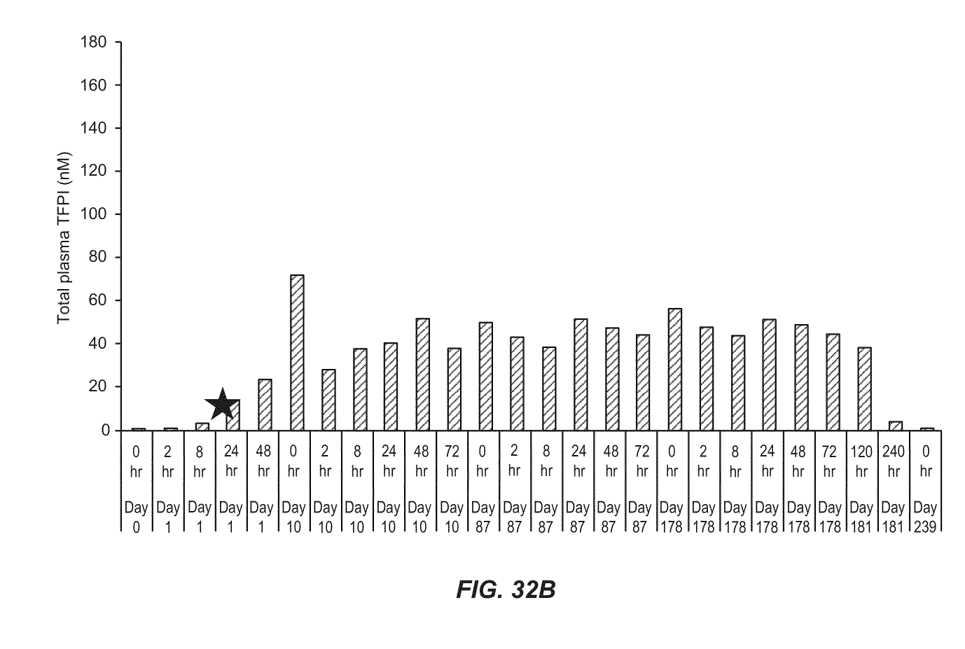

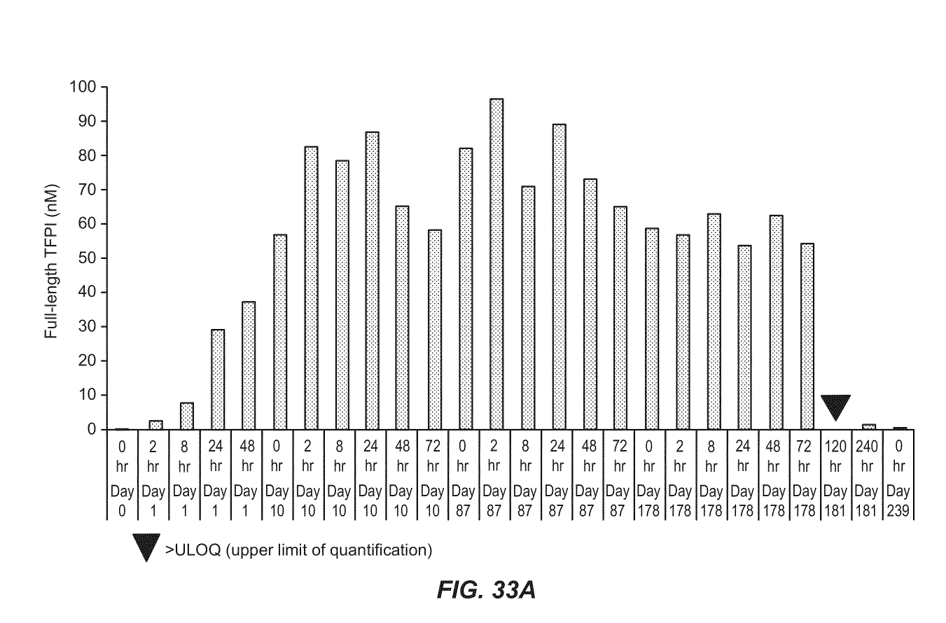

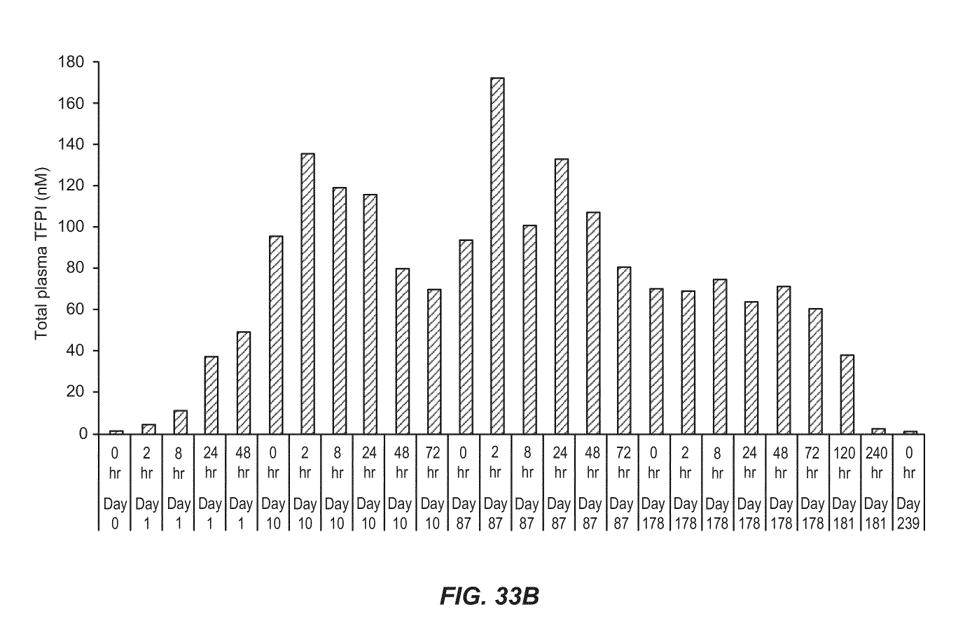

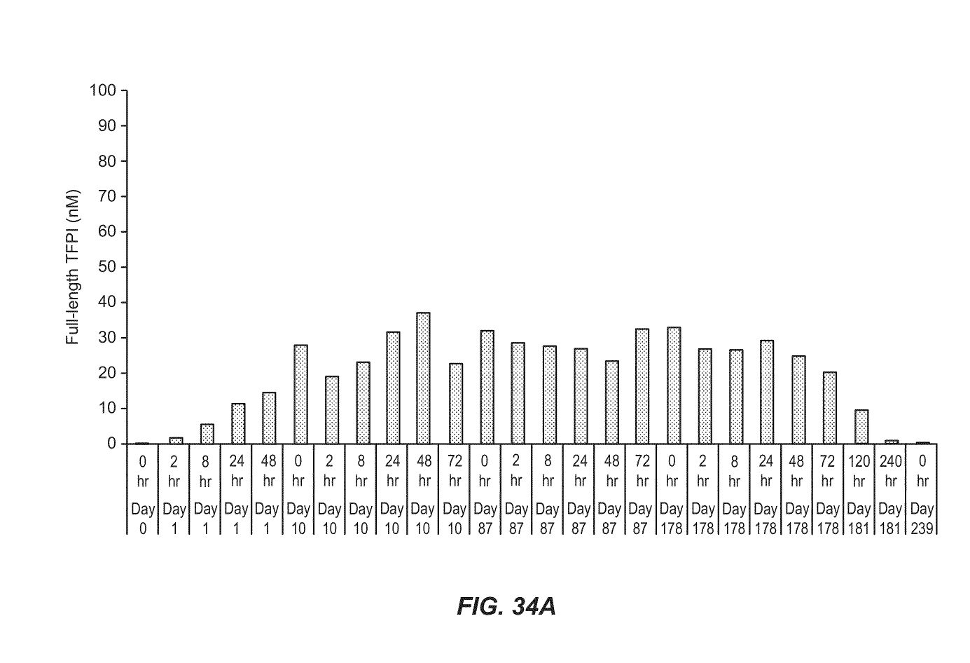

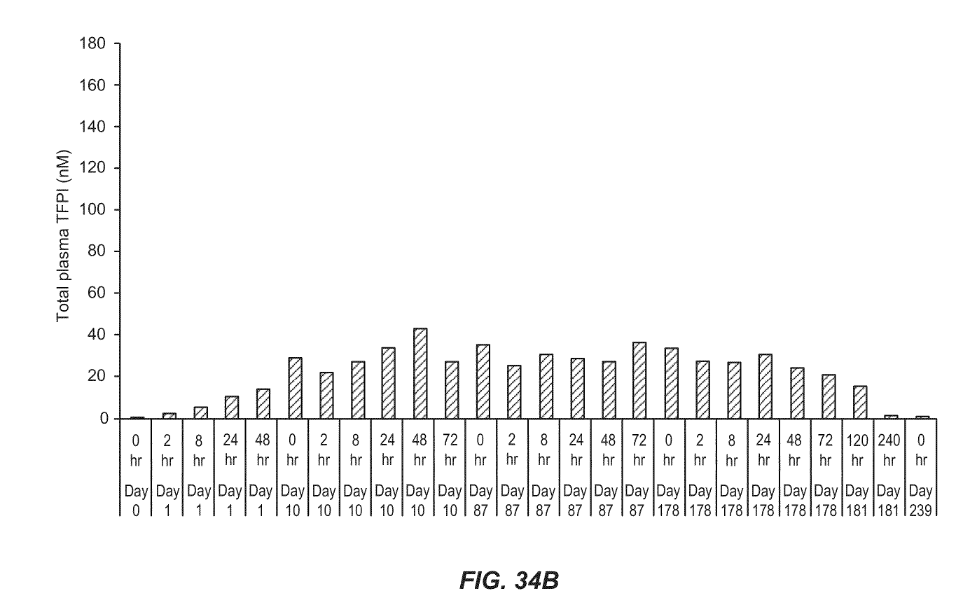

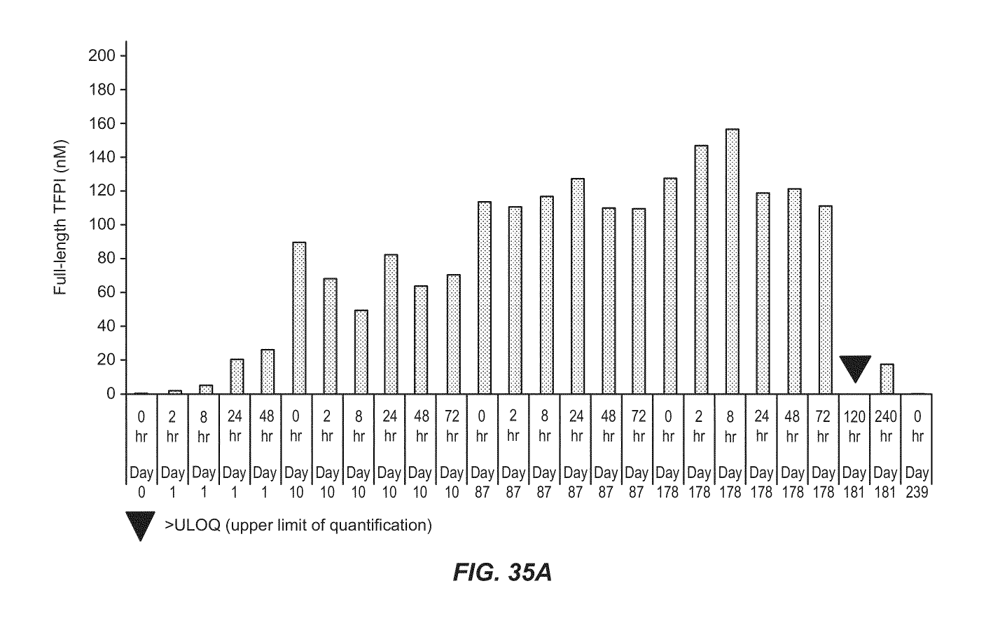

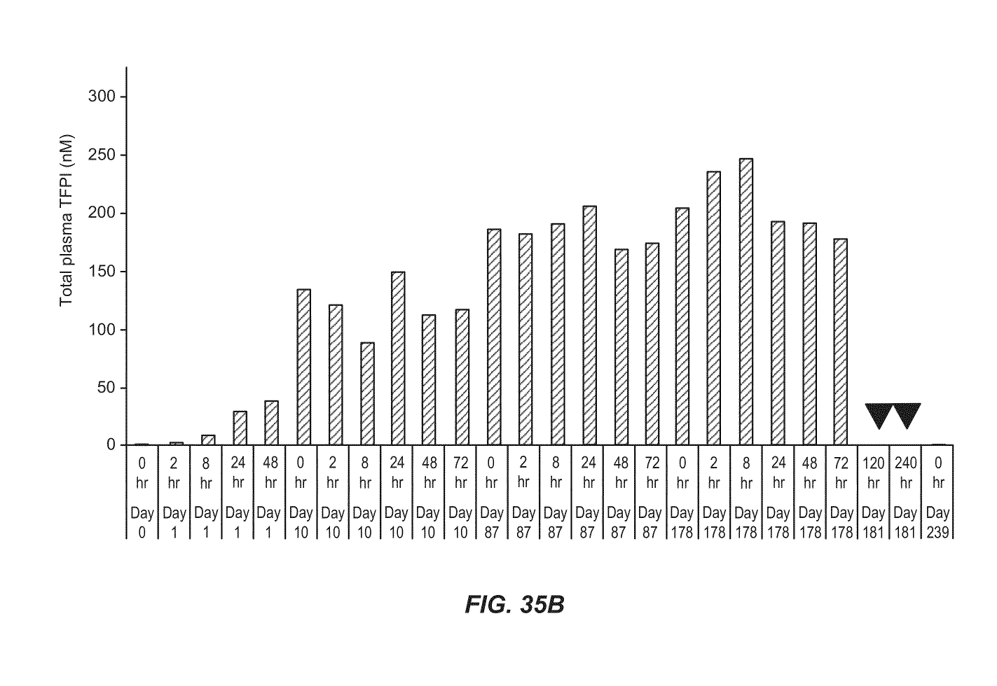

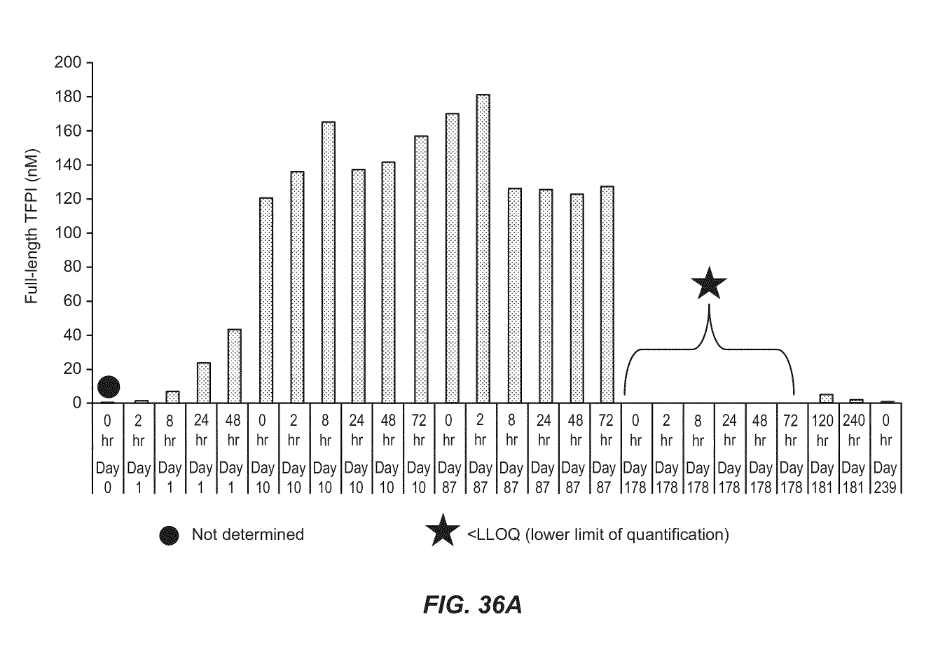

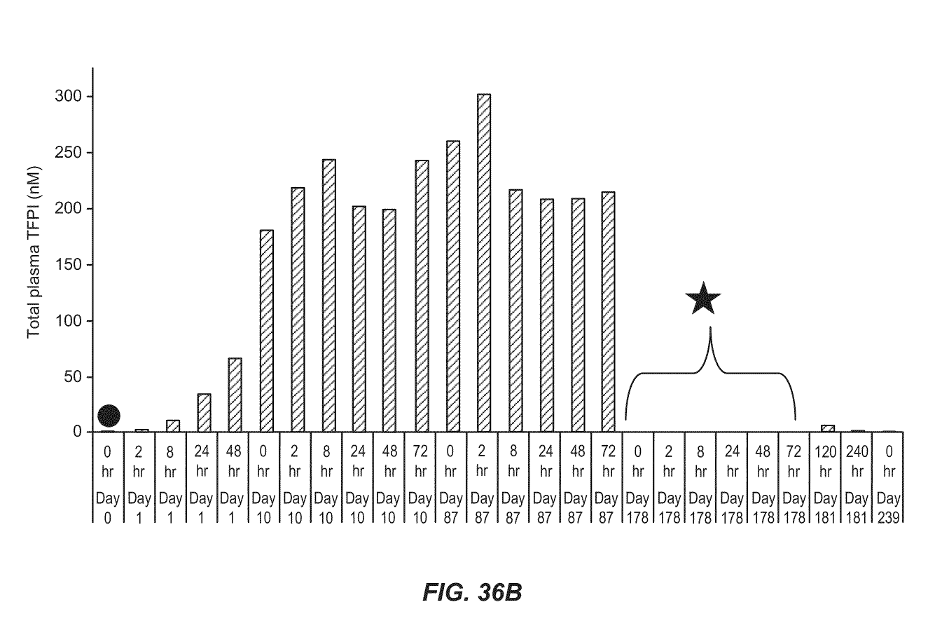

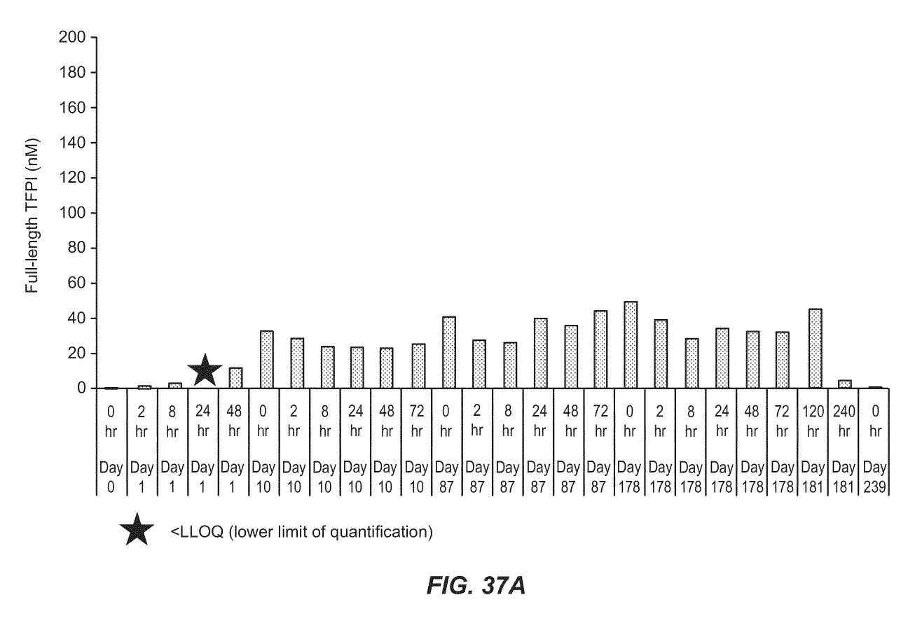

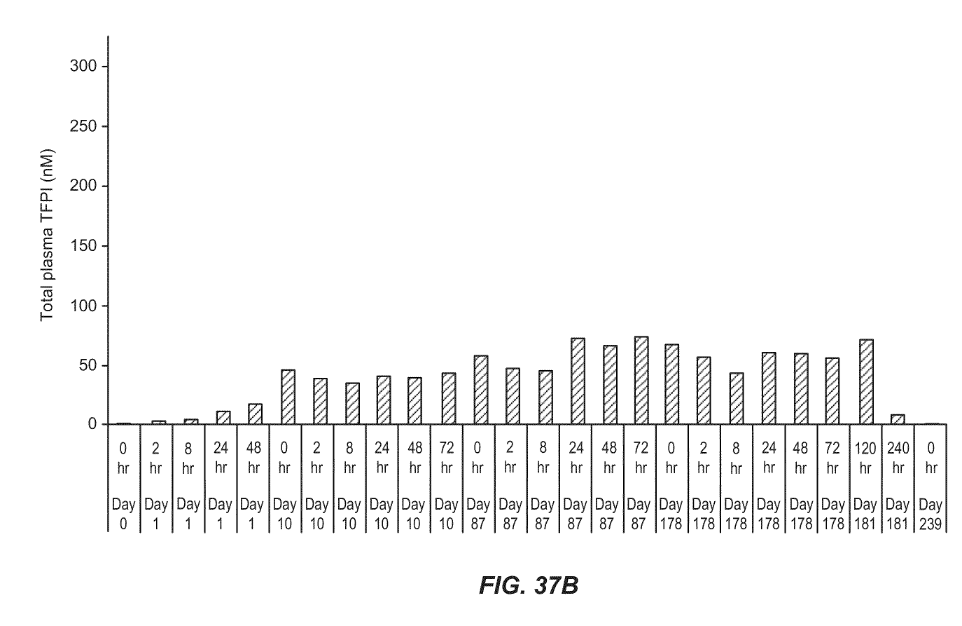

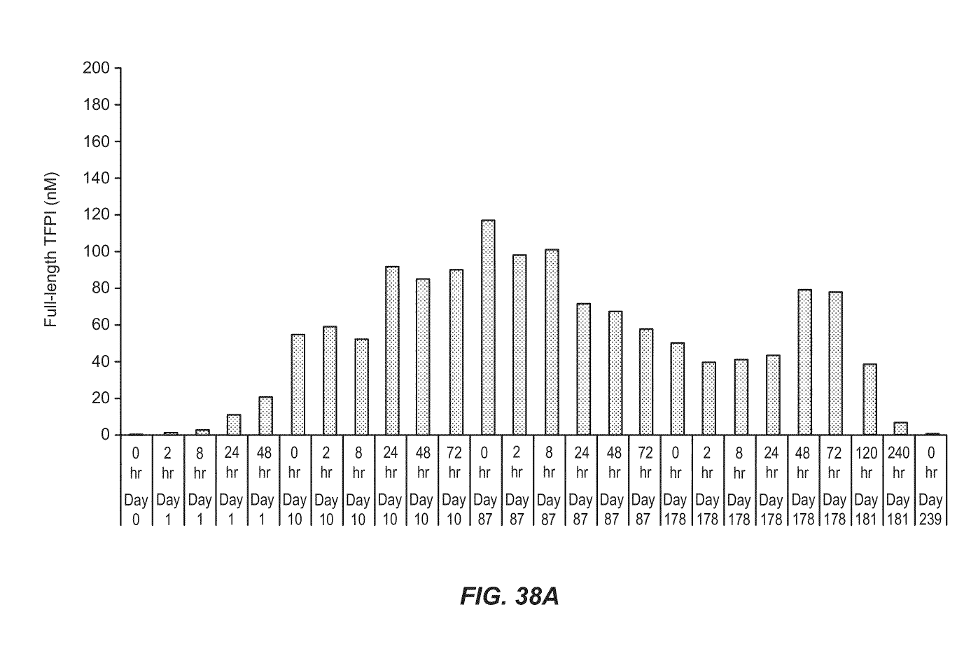

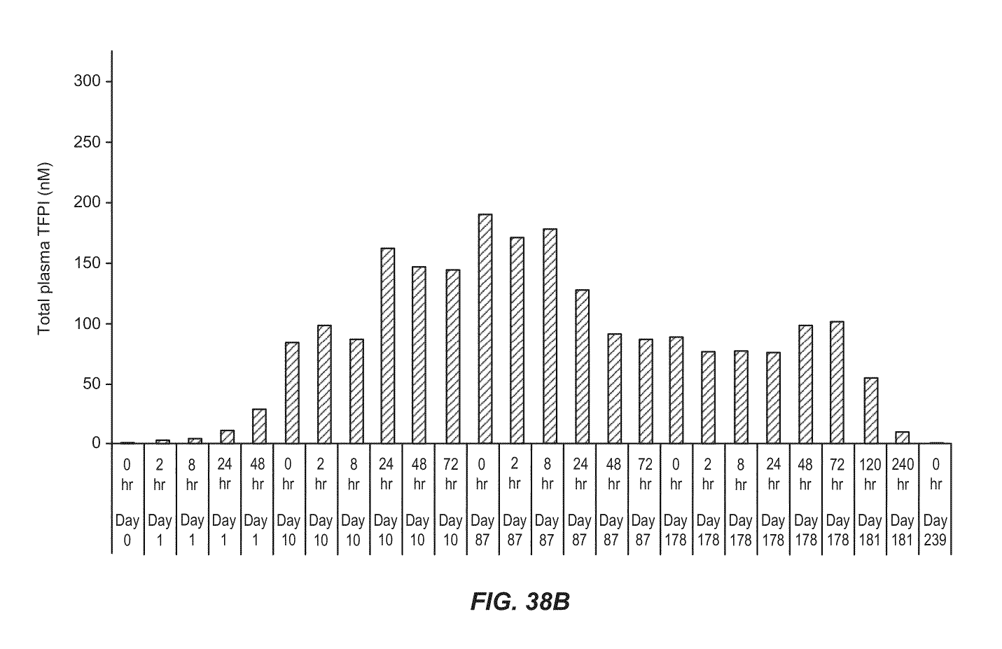

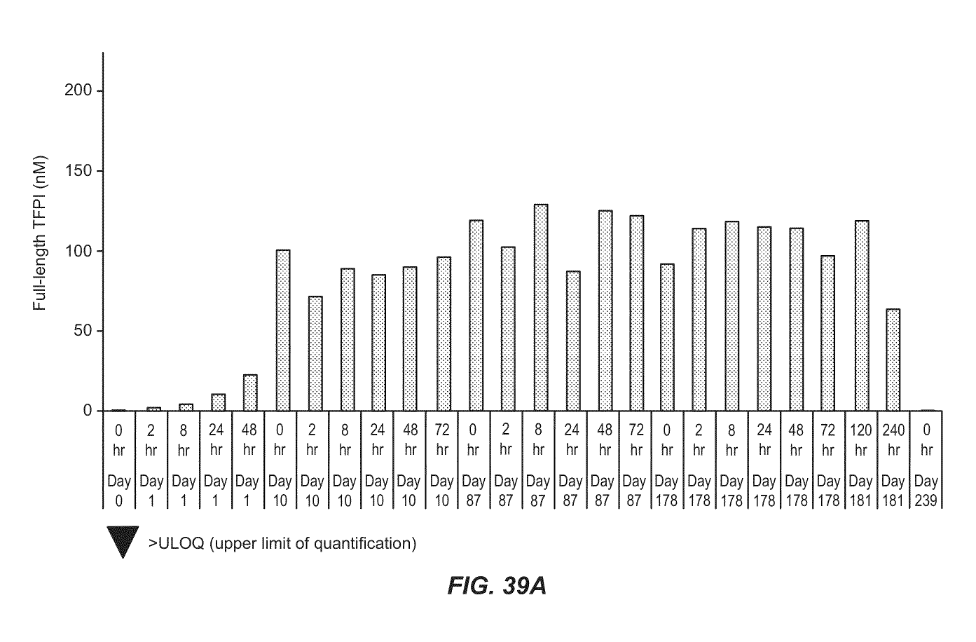

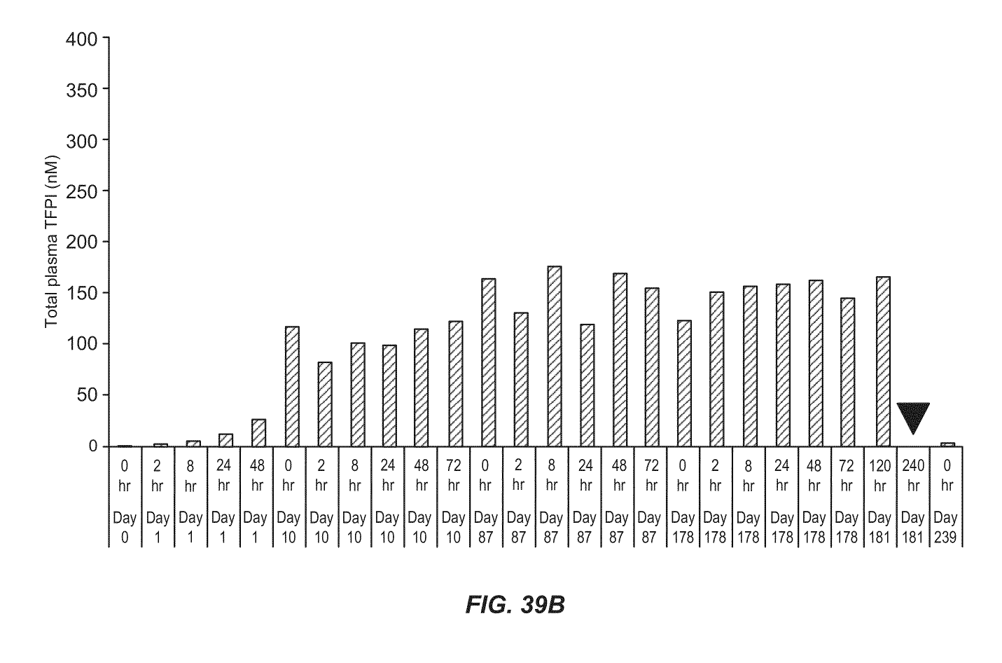

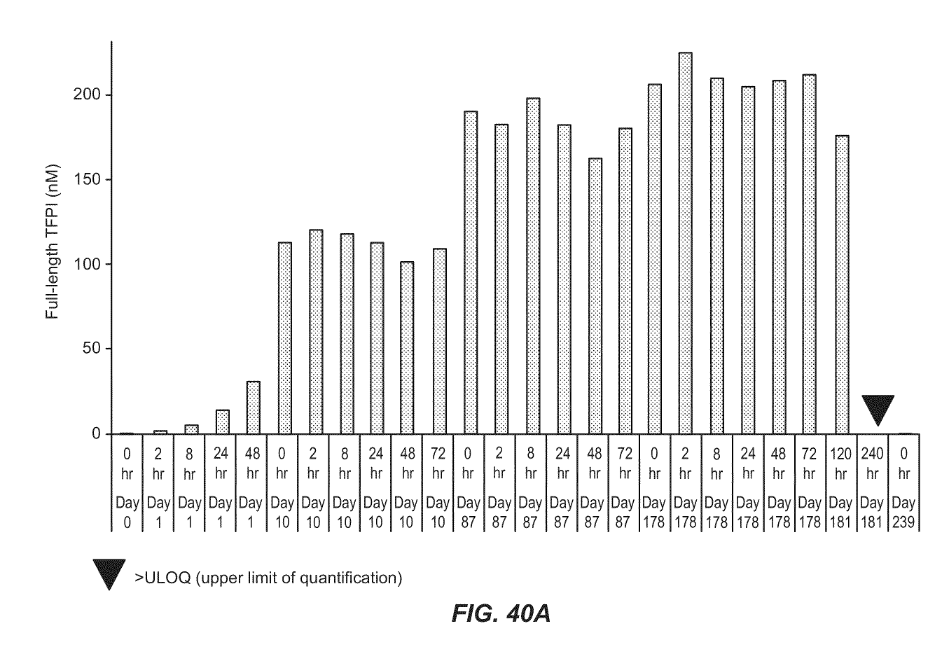

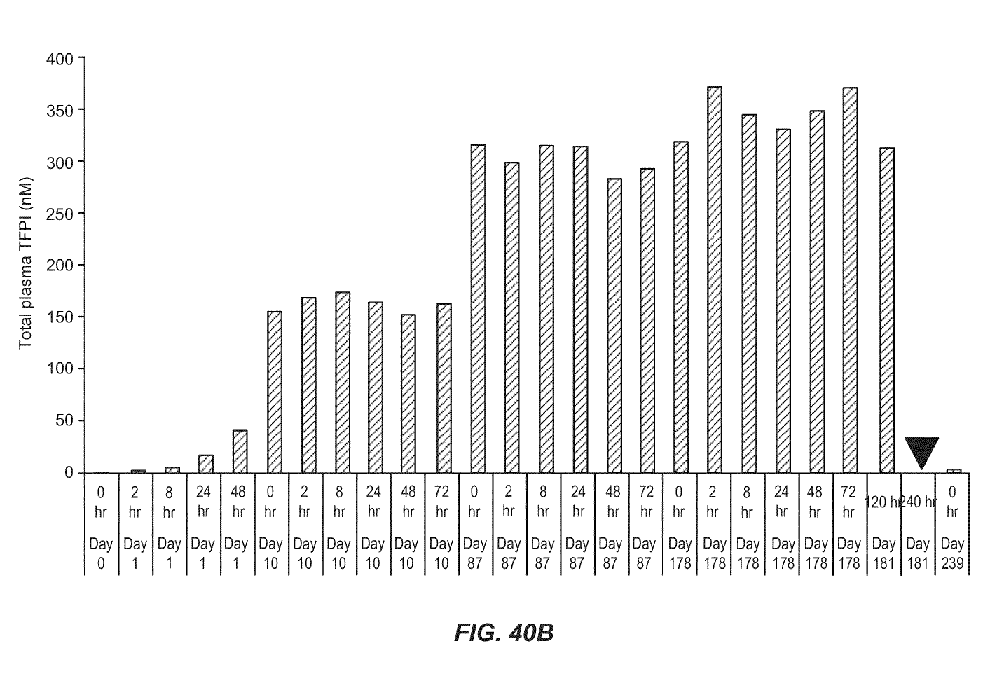

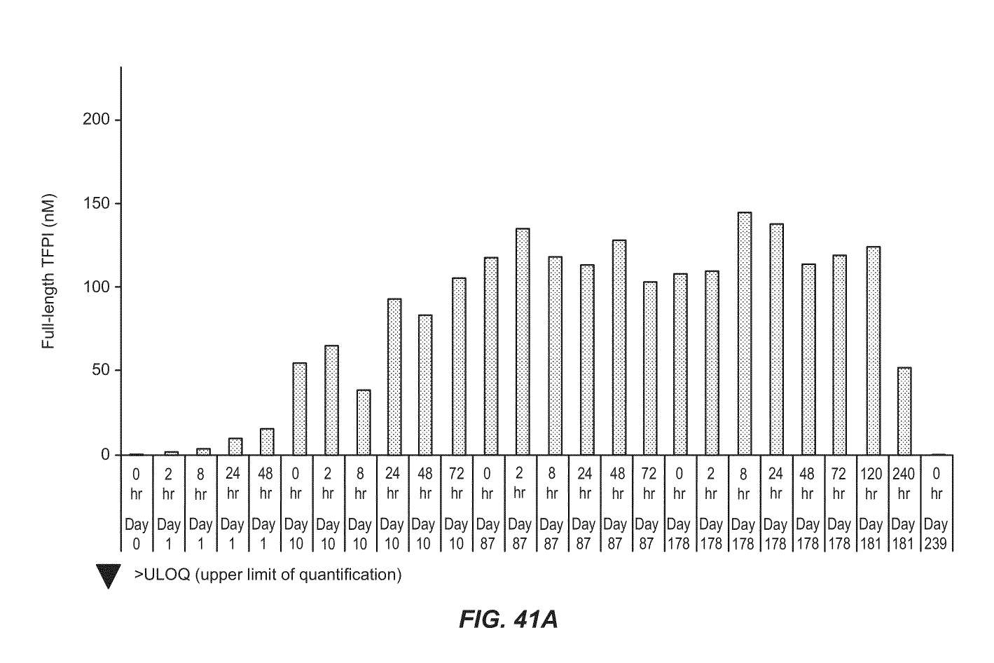

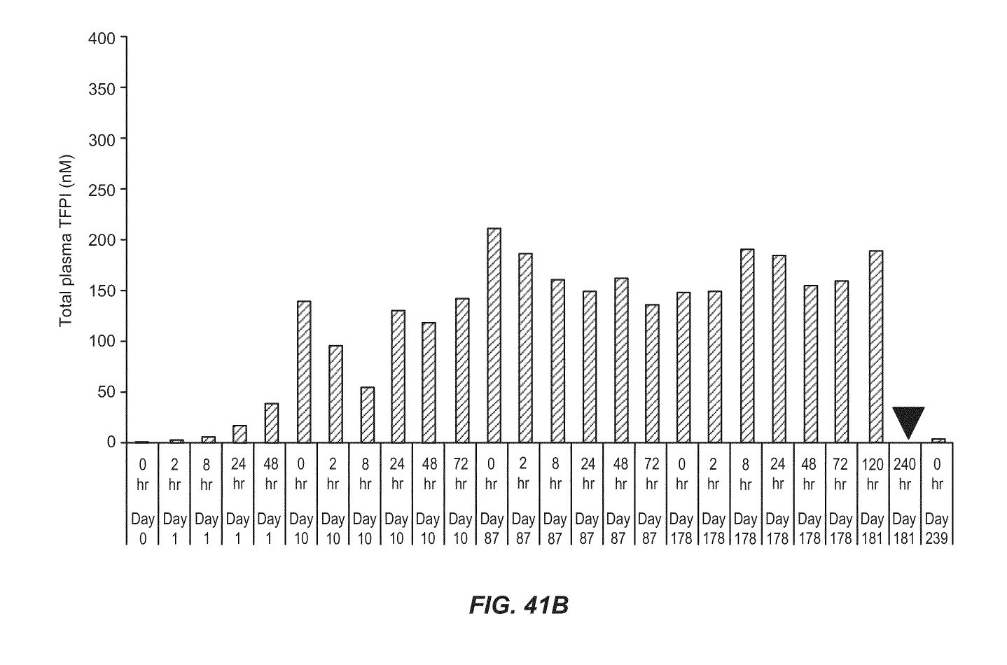

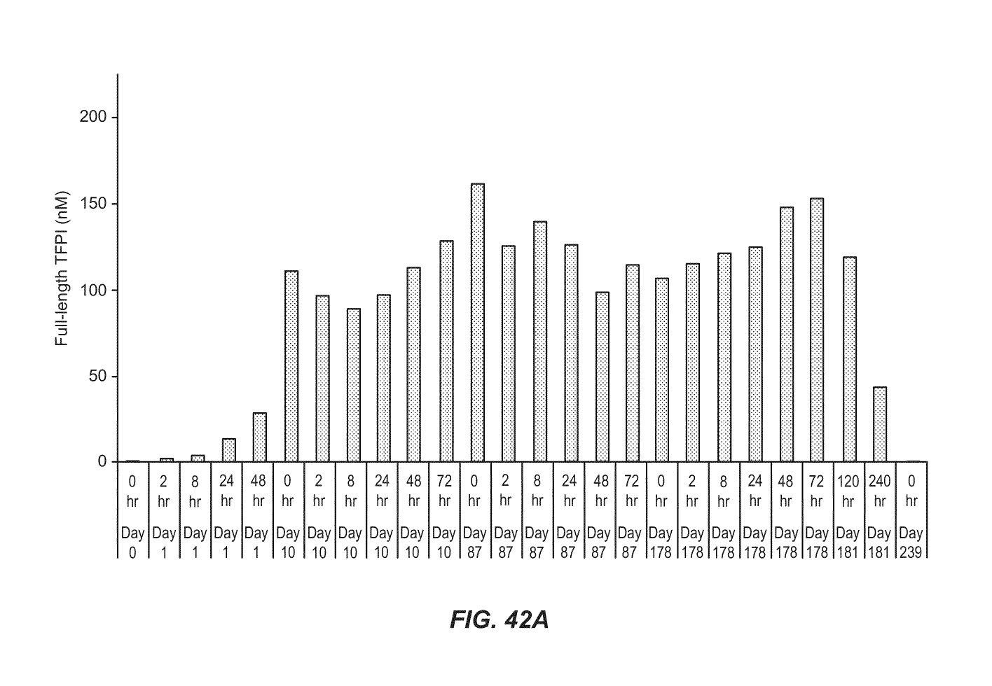

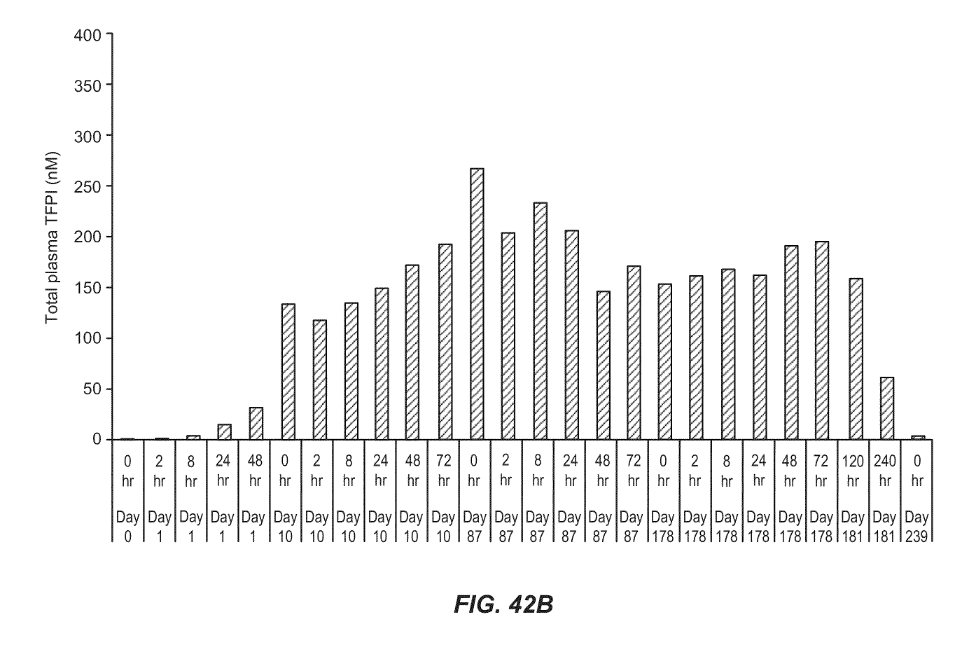

The total and full-length TFPI concentrations were measured in plasma samples from 4 groups of monkeys. The measured TFPI concentrations in samples from Group 1 monkeys are shown in Table 3 and FIG. 23; those from Group 2 monkeys are shown in Table 4 and FIG. 24. The measured TFPI concentrations in samples from monkeys in Groups 3 and 4 are shown in Table 5 and FIG. 25, and Table 6 and FIG. 26, respectively. Additionally, graphs of the TFPI concentrations for plasma samples from individual monkeys are shown in FIG. 27 through FIG. 42.

TABLE 3
TFPI concentration (nM) for individual monkeys in Group 1 (Vehicle Control)

| | | 1006-M | | 1105-M | | 1505-F | | 1506-F | |
|---|---|---|---|---|---|---|---|---|---|
| | | | Full- | | Full- | | Full- | | Full- |
| | | Total | length | Total | length | Total | length | Total | length |
| Week-1 | 0 hr | 0.88 | 0.25 | n.d* | n.d | 0.74 | 0.08 | 0.88 | 0.12 |
| Day 1 | 2 hr | 0.71 | 0.21 | 0.61 | 0.12 | 0.69 | 0.11 | 0.67 | 0.12 |
| | 8 hr | 0.70 | 0.21 | 0.60 | 0.14 | 0.63 | 0.09 | 0.62 | 0.11 |
| | 24 hr | 0.74 | 0.20 | 0.64 | 0.13 | 0.66 | 0.11 | 0.65 | 0.09 |
| | 48 hr | 0.78 | 0.22 | 0.72 | 0.15 | 0.69 | 0.11 | 0.79 | 0.09 |
| Day 10 | 0 hr | 0.79 | 0.22 | 0.88 | 0.16 | 0.87 | 0.10 | 0.91 | 0.08 |
| | 2 hr | 0.79 | 0.19 | 0.85 | 0.18 | 0.76 | 0.08 | 0.76 | 0.09 |
| | 8 hr | 0.79 | 0.21 | 0.49 | 0.16 | 0.73 | 0.11 | 0.83 | 0.10 |
| | 24 hr | 0.79 | 0.20 | <0.15# | 0.13 | 0.71 | 0.10 | 0.79 | 0.10 |
| | 48 hr | 0.85 | 0.19 | 0.47 | 0.12 | 0.78 | 0.10 | 1.07 | 0.12 |
| | 72 hr | 0.87 | 0.21 | 0.38 | 0.17 | 0.88 | 0.12 | 1.07 | 0.14 |
| Day 87 | 0 hr | 1.01 | 0.25 | 0.73 | 0.17 | 0.83 | 0.12 | 1.04 | 0.15 |
| | 2 hr | 0.85 | 0.23 | 0.73 | 0.18 | 0.72 | 0.10 | 0.94 | 0.15 |
| | 8 hr | 0.80 | 0.23 | 0.67 | 0.18 | 0.70 | 0.12 | 0.96 | 0.13 |
| | 24 hr | 0.75 | 0.20 | 0.58 | 0.11 | 0.62 | 0.09 | 0.95 | 0.10 |
| | 48 hr | 0.90 | 0.23 | 0.55 | 0.13 | 0.62 | 0.08 | 0.67 | 0.07 |
| | 72 hr | 0.87 | 0.23 | 0.51 | 0.12 | 0.71 | 0.08 | 0.69 | 0.09 |
| Day 178 | 0 hr | 0.93 | 0.24 | 0.21 | 0.14 | 0.83 | 0.10 | 0.87 | 0.11 |
| | 2 hr | 0.79 | 0.19 | 0.79 | 0.16 | 0.75 | 0.10 | 0.65 | 0.09 |
| | 8 hr | 0.72 | 0.21 | 0.23 | 0.16 | 0.72 | 0.12 | 0.65 | 0.10 |
| | 24 hr | 0.71 | 0.18 | 0.32 | 0.11 | 0.67 | 0.08 | 0.65 | 0.07 |
| | 48 hr | 0.86 | 0.20 | 0.70 | 0.13 | 0.82 | 0.08 | 0.77 | 0.09 |
| | 72 hr | 0.79 | 0.20 | 0.71 | 0.16 | 0.68 | 0.09 | 0.40 | 0.08 |
| Day 181 | 120 hr | 0.86 | 0.21 | 0.83 | 0.18 | 0.77 | 0.10 | 0.81 | 0.09 |
| | 240 hr | 0.95 | 0.21 | 0.47 | 0.16 | 0.75 | 0.10 | 0.71 | 0.13 |
| Day 239 | 0 hr | 0.80 | 0.21 | 0.69 | 0.13 | 0.66 | 0.067 | 0.38 | 0.07 |

| | | | | | | | | |
|---|---|---|---|---|---|---|---|---|
| Mean | 0.82 | 0.21 | 0.60 | 0.15 | 0.73 | 0.10 | 0.78 | 0.12 |
| concentration | | | | | | | | |
| SD of mean | 0.078 | 0.018 | 0.185 | 0.023 | 0.072 | 0.015 | 0.178 | 0.023 |

*n.d: not determined (sample was not available);
#Lower limit of quantification (LLOQ) for the dilution employed

TABLE 4
TFPI concentration (nM) for individual monkeys in Group 2(1.25 mg/kg BAX499)

| | | 2005-M | | 2006-M | | 2505-F | | 2506-F | |
|---|---|---|---|---|---|---|---|---|---|
| | | | Full- | | Full- | | Full- | | Full- |
| | | Total | length | Total | length | Total | length | Total | length |
| Week-1 | 0 hr | 0.72 | 0.09 | 0.48 | 0.16 | 0.76 | 0.18 | 0.64 | 0.11 |
| Day 1 | 2 hr | 1.43 | 1.57 | 0.49 | 0.71 | 4.36 | 2.49 | 2.50 | 1.45 |
| | 8 hr | 3.11 | 3.33 | 3.31 | 3.61 | 10.73 | 7.63 | 5.73 | 5.48 |
| | 24 hr | <9.30* | 8.48 | 14.00 | 14.00 | 36.73 | 29.07 | 10.64 | 11.65 |
| | 48 hr | 19.13 | 16.91 | 23.69 | 23.56 | 49.34 | 37.08 | 14.54 | 14.80 |
| Day 10 | 0 hr | 83.80 | 53.02 | 72.12 | 45.05 | 95.38 | 56.78 | 28.87 | 27.74 |
| | 2 hr | 33.50 | 36.00 | 28.11 | 31.42 | 135.65 | 82.20 | 22.16 | 18.98 |
| | 8 hr | 54.52 | 48.81 | 37.89 | 41.05 | 119.41 | 78.42 | 27.27 | 22.88 |
| | 24 hr | 38.82 | 42.12 | 40.69 | 38.85 | 115.45 | 86.75 | 34.48 | 31.61 |
| | 48 hr | 44.67 | 45.99 | 51.89 | 49.44 | 79.62 | 64.82 | 43.69 | 36.99 |
| | 72 hr | 53.36 | 44.95 | 37.89 | 39.26 | 70.22 | 58.01 | 27.27 | 22.39 |
| Day 87 | 0 hr | 52.21 | 48.07 | 50.02 | 44.48 | 93.84 | 81.86 | 35.90 | 32.28 |
| | 2 hr | 54.52 | 37.65 | 43.02 | 44.62 | 172.63 | 96.31 | 25.81 | 28.46 |
| | 8 hr | 55.66 | 48.37 | 38.36 | 43.93 | 101.35 | 70.63 | 30.89 | 27.80 |
| | 24 hr | 47.00 | 38.25 | 51.42 | 41.74 | 133.02 | 88.82 | 28.72 | 26.81 |
| | 48 hr | 44.67 | 39.59 | 47.22 | 42.70 | 107.09 | 72.66 | 27.27 | 23.20 |
| | 72 hr | 37.64 | 33.45 | 43.96 | 43.39 | 80.51 | 65.27 | 36.62 | 32.45 |
| Day 178 | 0 hr | 51.64 | 51.20 | 56.56 | 52.76 | 69.77 | 58.89 | 33.76 | 33.12 |
| | 2 hr | 44.67 | 38.25 | 47.69 | 48.61 | 68.87 | 56.85 | 27.99 | 26.81 |
| | 8 hr | 52.21 | 51.64 | 43.49 | 48.34 | 73.81 | 63.02 | 26.54 | 26.48 |
| | 24 hr | 44.09 | 41.83 | 51.42 | 46.69 | 63.02 | 53.54 | 30.89 | 29.29 |
| | 48 hr | 37.05 | 30.42 | 49.09 | 50.82 | 70.67 | 62.28 | 24.35 | 24.84 |
| | 72 hr | 41.75 | 45.40 | 44.89 | 47.65 | 59.41 | 54.25 | 20.69 | 20.11 |
| Day 181 | 120 hr | 31.07 | 17.39 | 38.33 | 18.61 | 37.78 | >18.61# | 15.44 | 9.43 |
| | 240 hr | 7.23 | 2.83 | 3.82 | 1.50 | 1.69 | 0.91 | 1.13 | 0.63 |
| Day 239 | 0 hr | 0.84 | 0.12 | 0.51 | 0.10 | 0.65 | 0.18 | 0.66 | 0.09 |

*Lower limit of quantification (LLOQ) for the dilution employed;
#Upper limit of quantification (ULOQ) for the dilution employed

TABLE 5
TFPI concentration (nM) for individual monkeys in Group 3(3.75 mg/kg BAX499)

| | | 3005-M | | 3106-M | | 3505-F | | 3506-F | |
|---|---|---|---|---|---|---|---|---|---|
| | | | Full- | | Full- | | Full- | | Full- |
| | | Total | length | Total | length | Total | length | Total | length |
| Week-1 | 0 hr | 1.27 | 0.12 | n.d.# | n.d | 0.53 | 0.09 | 0.86 | 0.07 |
| Day 1 | 2 hr | 2.96 | 1.43 | 2.08 | 0.97 | 2.62 | 1.56 | 2.71 | 1.51 |
| | 8 hr | 8.66 | 5.13 | 11.33 | 6.97 | 4.23 | 2.91 | 5.29 | 3.15 |
| | 24 hr | 29.56 | 20.52 | 35.08 | 22.85 | 10.18 | <9.30† | 12.91 | 10.12 |
| | 48 hr | 38.58 | 25.92 | 65.97 | 43.26 | 16.90 | 11.42 | 29.56 | 20.52 |
| Day 10 | 0 hr | 135.33 | 89.46 | 180.54 | 120.64 | 45.46 | 32.57 | 84.71 | 54.60 |
| | 2 hr | 122.09 | 68.06 | 218.29 | 135.77 | 38.10 | 28.80 | 98.61 | 59.38 |
| | 8 hr | 89.18 | 49.41 | 243.77 | 165.03 | 34.91 | 24.03 | 87.90 | 52.27 |
| | 24 hr | 150.28 | 82.07 | 201.61 | 137.17 | 40.22 | 23.16 | 162.39 | 91.43 |
| | 48 hr | 113.31 | 63.73 | 198.65 | 141.37 | 39.16 | 22.73 | 146.19 | 84.81 |
| | 72 hr | 117.14 | 70.80 | 243.77 | 156.70 | 42.84 | 25.34 | 145.03 | 89.97 |
| Day 87 | 0 hr | 187.31 | 113.36 | 259.88 | 169.88 | 57.25 | 40.81 | 190.40 | 116.69 |
| | 2 hr | 183.24 | 110.72 | 302.33 | 180.95 | 45.97 | 27.50 | 172.27 | 98.00 |
| | 8 hr | 192.11 | 117.13 | 217.69 | 125.91 | 44.41 | 25.77 | 179.85 | 100.90 |
| | 24 hr | 206.94 | 126.92 | 208.73 | 125.20 | 72.17 | 39.53 | 128.31 | 71.39 |
| | 48 hr | 170.68 | 109.96 | 208.73 | 122.37 | 65.76 | 36.11 | 90.93 | 66.91 |
| | 72 hr | 175.48 | 109.58 | 214.69 | 127.32 | 72.66 | 44.21 | 87.61 | 57.30 |
| Day 178 | 0 hr | 205.83 | 127.29 | <17.44^{†} | <17.44^{†} | 66.76 | 49.29 | 89.51 | 50.14 |
| | 2 hr | 238.83 | 147.15 | <17.44^{†} | <17.44^{†} | 55.73 | 39.10 | 75.59 | 39.53 |
| | 8 hr | 248.75 | 156.50 | <17.44^{†} | <17.44^{†} | 42.32 | 28.37 | 78.02 | 41.23 |
| | 24 hr | 194.33 | 118.26 | <17.44^{†} | <17.44^{†} | 59.27 | 34.39 | 76.56 | 42.94 |
| | 48 hr | 192.85 | 121.28 | <17.44^{†} | <17.44^{†} | 59.27 | 32.25 | 98.33 | 78.78 |
| | 72 hr | 179.55 | 111.09 | <17.44^{†} | <17.44^{†} | 55.22 | 32.25 | 101.35 | 77.85 |
| Day 181 | 120 hr | >74.42* | >72.42* | 7.28 | 4.97 | 70.88 | 45.44 | 55.18 | 38.42 |
| | 240 hr | >18.61* | 17.64 | 1.76 | 0.89 | 7.63 | 4.64 | 9.88 | 6.39 |
| Day 239 | 0 hr | 1.69 | 0.23 | 0.66 | 0.09 | 0.49 | 0.11 | 0.66 | 0.15 |

*Upper limit of quantification (ULOQ) for the dilution employed;
#n.d: not determined;
^{†}Lower limit of quantification (LLOQ) for the dilution employed

TABLE 6
TFPI concentration (nM) for individual monkeys in Group 4(12.5 mg/kg BAX499)

| | | 4005-M | | 4006-M | | 4505-F | | 4506-F | |
|---|---|---|---|---|---|---|---|---|---|
| | | | Full- | | Full- | | Full- | | Full- |
| | | Total | length | Total | length | Total | length | Total | length |
| Week-1 | 0 hr | 0.36 | 0.12 | 0.57 | 0.12 | 0.57 | 0.11 | 1.81 | 0.03 |
| Day 1 | 2 hr | 0.60 | 0.31 | 1.69 | 1.05 | 1.40 | 0.81 | 1.30 | 0.78 |
| | 8 hr | 4.16 | 3.85 | 7.24 | 5.24 | 4.68 | 3.73 | 4.23 | 3.41 |
| | 24 hr | 11.39 | 10.38 | 17.65 | 14.11 | 16.42 | 9.90 | 15.16 | 12.90 |
| | 48 hr | 24.81 | 22.60 | 42.36 | 30.71 | 38.23 | 16.15 | 31.63 | 28.23 |
| Day 10 | 0 hr | 116.95 | 100.37 | 155.45 | 112.78 | 138.43 | 54.67 | 133.42 | 110.89 |
| | 2 hr | 80.98 | 71.53 | 168.79 | 119.99 | 95.98 | 66.10 | 117.24 | 96.21 |
| | 8 hr | 100.24 | 88.60 | 174.90 | 118.96 | 54.15 | 39.15 | 134.84 | 88.85 |
| | 24 hr | 97.11 | 84.57 | 166.25 | 113.26 | 129.75 | 93.44 | 149.00 | 96.92 |
| | 48 hr | 113.21 | 89.95 | 152.54 | 100.65 | 118.15 | 84.12 | 172.35 | 112.74 |
| | 72 hr | 120.37 | 96.21 | 162.69 | 108.56 | 142.26 | 106.37 | 192.26 | 128.70 |
| Day 87 | 0 hr | 162.97 | 119.03 | 315.72 | 190.12 | 210.79 | 118.45 | 267.03 | 161.26 |
| | 2 hr | 130.25 | 102.47 | 298.98 | 182.33 | 186.62 | 136.22 | 203.54 | 125.64 |
| | 8 hr | 175.02 | 128.90 | 316.26 | 198.37 | 160.25 | 118.81 | 232.99 | 139.84 |
| | 24 hr | 119.03 | 87.26 | 315.17 | 181.84 | 147.95 | 114.17 | 206.85 | 126.01 |
| | 48 hr | 169.90 | 125.76 | 284.00 | 162.24 | 162.32 | 128.55 | 146.32 | 98.66 |
| | 72 hr | 154.26 | 121.72 | 294.15 | 179.89 | 136.59 | 104.26 | 171.88 | 114.52 |
| Day 178 | 0 hr | 121.72 | 91.74 | 319.52 | 206.13 | 148.36 | 108.49 | 153.27 | 106.73 |
| | 2 hr | 150.61 | 113.66 | 373.29 | 225.04 | 148.77 | 110.61 | 161.08 | 115.24 |
| | 8 hr | 155.63 | 118.13 | 345.87 | 209.52 | 190.45 | 145.84 | 168.13 | 120.96 |
| | 24 hr | 157.46 | 114.55 | 331.54 | 204.68 | 184.50 | 138.80 | 162.25 | 125.02 |
| | 48 hr | 161.59 | 113.66 | 349.20 | 208.07 | 154.91 | 114.52 | 190.78 | 147.97 |
| | 72 hr | 142.89 | 96.66 | 372.72 | 211.95 | 159.84 | 119.89 | 194.98 | 153.64 |
| Day 181 | 120 hr | 165.29 | 118.29 | 314.06 | 175.53 | 189.44 | 125.01 | 158.98 | 119.34 |
| | 240 hr | >74.42* | 63.13 | >74.42* | >74.42* | >74.42* | 52.30 | 62.02 | 43.01 |
| Day 239 | 0 hr | 2.41 | 0.11 | 1.18 | 0.46 | 0.92 | 0.48 | 0.87 | 0.24 |

*Upper limit of quantification (ULOQ) for the dilution employed

The baseline samples, as well as the samples from the monkeys in Group 1, were measured with a much lower dilution (1:25 for total TFPI and 1:10 for full-length TFPI), allowing for more accurate comparisons of TFPI concentrations among these samples. A baseline of plasma TFPI concentrations in cynomolgus monkeys can be established with Week −1 samples from the 14 animals (see Week −1, 0 hr values in Table 3 through Table 6). Mean total TFPI concentrations±standard deviation (SD) were 0.79±0.369 nM; mean full-length TFPI concentrations were 0.12±0.053 nM. Concentrations of measured full-length TFPI were generally ˜8 to 33% (mean: 18%) of measured total TFPI in the baseline samples for each individual monkey.

As shown in FIG. 23, monkeys that received vehicle control had both full-length and total plasma TFPI concentrations that remained relatively constant over the course of the study. Mean and SD for individual animals are presented in Table 3. These values are consistent with the baseline values.

In contrast, monkeys that received BAX 499 (Groups 2 to 4) showed elevated TFPI concentrations throughout the study. Plasma TFPI concentrations (both full-length and total) increased in a time-dependent manner during Day 1 (2 to 48 hours post dose). By Day 10, a plateau had been reached; the precise time at which the plateau was initially reached cannot be determined without data points between Days 1 and 10. Thereafter, concentrations of TFPI remained relatively unchanged through Days 87 and 178. For individual animals, TFPI concentrations fluctuated between time points; these fluctuations may be a result of error associated with the extent of dilution required for sample measurement and/or may reflect inherent variability in TFPI concentrations in monkeys. In BAX 499-treated monkeys, TFPI concentrations decreased after the Day 181 dose (120 and 240 hr post-dose) and by Day 239, after an 8-week washout period, were near the pre-dose baseline levels (see FIG. 24 for Group 2, FIG. 25 for Group 3, and FIG. 26 for Group 4). This trend was seen for most monkeys except Group 3 monkey 3106-M; plasma samples from this monkey had undetectable concentrations of both full-length and total TFPI on Day 178, and very low concentrations on Day 181 compared to the other Group 3 monkeys (7.28 and 4.97 nM total and full-length TFPI, respectively, compared to >55 nM total TFPI and >38 nM full-length TFPI for the other Group 3 monkeys (Table 5)). Sample analysis for monkey 3106-M was repeated with similar results; the cause of the low TFPI concentrations in this monkey is currently unclear.

In most measured samples from monkeys that received BAX 499 (Groups 2, 3, and 4), concentrations of full-length TFPI increased to a greater extent than those of total TFPI when compared to the relevant baseline sample. For the majority of samples from Days 10, 87, and 178, the measured concentrations of full-length TFPI were ˜60-90% of the measured corresponding total TFPI concentration (Table 4 through Table 6). This is in contrast to the Group 1 (vehicle control) monkeys, for which the measured full-length TFPI concentrations were ˜8-33% of the measured total TFPI concentrations. Upon completion of dosing, the percent full-length TFPI gradually decreased and reached baseline by Day 239. These results suggest that the increase in total TFPI concentration is due to the increase in full-length TFPI.

TFPI concentrations varied among monkeys within each group that received BAX 499 (Table 4 through Table 6; FIG. 24 through FIG. 26). At the plateau (i.e., Days 10, 87, and 178), mean concentrations of total TFPI were 54.9±29.70, 133.8±70.68, and 182.7±73.29 nM for Groups 2, 3, and 4, respectively. Similarly, mean concentrations of full-length TFPI were 46.1±17.18, 82.7±44.45, and 124.6±38.88 nM. The overall concentrations of both total and full-length TFPI were lower in samples from the Group 2 monkeys than in samples from Groups 3 and 4, and may also trend higher in samples from monkeys in Group 4 versus Group 3. However, due to the large standard deviation associated with each group mean, no relationship between TFPI concentration and BAX 499 dose can be discerned. No sex-related difference in the increase in TFPI concentrations was apparent in plasma samples from males versus females in each group.

Both full-length and total TFPI concentrations in samples from Group 1 monkeys remained constant over the course of the study, and these samples all had more total than full-length plasma TFPI. For the groups that received BAX 499, the concentrations of both full-length and total plasma TFPI increased in a time-dependent manner after the first dose on Day 1, reached equilibrium by Day 10, and remained relatively unchanged through Day 178. The measured TFPI concentrations in BAX 499-treated monkeys decreased by 120 hours after the final dose at Day 181 and were near baseline levels in the samples at Day 239 (end of recovery period). Full-length TFPI concentrations increased more dramatically than total TFPI concentrations when compared to baseline levels. This finding indicates that the increase in total TFPI concentrations was due largely to the increase in full-length TFPI concentration. The overall concentrations of full-length and total TFPI were higher in samples from the Group 3 and Group 4 monkeys when compared to samples from the lower-dosed Group 2 monkeys.

### Example 2

### Human Phase I Clinical Trials Demonstrated that BAX 499 Leads to Unexpected Anti-Coagulant Activity

A human Phase I clinical trial sought to determine safety and pharmacokinetics in hemophilia patients. The trial used a population of hemophilia A and B patients with all severities. The objectives were to assess pharmacokinetics of BAX 499 after single and multiple doses by intravenous and subcutaneous routes of administration, to assess pharmacodynamics of BAX 499 using various assays of coagulation, and to evaluate the safety, tolerability, and immunogenicity of BAX 499. The clinical design was a randomized, double-blind, placebo-controlled, dose escalation study in male hemophilia patients, aged 18-75 years, and conducted in three parts.

Part A was single ascending dose (SAD). N=6, 3 dose levels: 3, 12, and 36 mg subcutaneous. Each subject randomized to 3 dose levels (2 active/1 placebo) in random order. Part B was subcutaneous to intravenous crossover. N=5, 4 subjects treated with 72 mg subcutaneous following by 36 mg intravenous (1 subject received placebo subcutaneous followed by placebo intravenous). Part C was multiple ascending dose (MAD). N=15, each subject randomized to 1 of 3 does cohorts (4 active/1 placebo). In each dose cohort, initial loading dose followed by 3 maintenance doses at 3 day intervals. Table 7 provides a detailed summary of the clinical trial design.

TABLE 7
Summary of human clinical trial design.

| | | |
|---|---|---|
| Part | N = 6, 3 dose levels: 3, 12 and 36 mg | Status: |
| A - Single | subcutaneous (SC) administration | 6 subject treated |
| Ascending | Each subject randomized to 3 dose | |
| Dose (SAD) | levels (2 active/1 placebo) in | |
| | random order | |
| Part B - SD | N = 5, 4 subjects treated with 72 mg | Status: |
| to | SC followed by 36 mg IV | 4 subjects treated |
| Intravenous | 1 subject received placebo SC | 1 inhibitor |
| (IV) | followed by placebo IV | subject non- |
| Crossover | | evaluable for |
| | | pharmacodynamics |
| | | (PD) |
| Part | N = 15, each subject randomized to | Status: |
| C - Multiple | 1 of 3 dose cohorts (4 active/1 | 7 subjects treated (6 |
| Ascending | placebo) | lose dose; 1 medium |
| Dose (MAD) | In each dose cohort, initial loading | dose) |
| | dose followed by 3 maintenance | |
| | doses at 3 day intervals | |
| | Low dose: 4.5 mg to 3 mg X3 | |
| | Medium dose: 45 mg to 30 mg | |
| | X3 | |

The study was discontinued after enrolling seventeen of twenty-two planned subjects due to a higher than expected incidence of bleeds observed in close temporal proximity to BAX 499 administration (within ten days). The bleed incidence and frequency were highest in the two highest dose cohorts (36 mg IV and 72 mg SC). Fifty percent (50%) of mild and moderate and sixty-six percent (66%) of severe hemophilia Z subjects experienced bleeds. The types of bleeds included hemarthroses, hematomas, bleeding from venipuncture sites and mucosal bleeding, all mild to moderate.

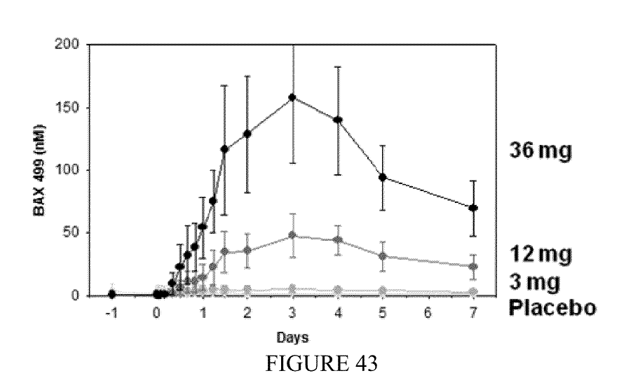

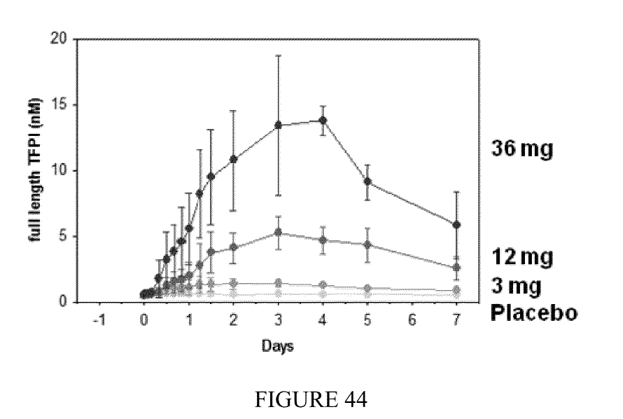

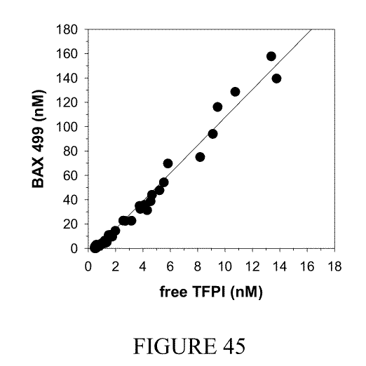

BAX 499 and TFPI levels in clinical trial subject blood samples (BAX 499 treated and control) were measured. FIGS. 43-45 illustrate data that were obtained from blood samples from subjects in Part A of the clinical trial. FIG. 43 is a graphical representation of the BAX 499 level over time in days from placebo (N=6) and BAX 499 treated (N=4) subject blood samples. A dose response was observed when increasing amounts of BAX 499 were dosed subcutaneously. FIG. 44 is a graphical representation of the TFPI level over time in days from placebo (N=6) and BAX 499 treated (N=4) subject blood samples. BAX 499 dose-dependently induced an increase in full length TFPI. It was also observed that full length TFPI levels followed the pharmacokinetics of BAX 499. FIG. 45 demonstrates the tight correlation between TFPI levels and BAX 499 concentration. The data were analyzed using a Spearman rank order correlation. Full length TFPI concentration was significantly correlated to BAX 499 level at any dose or sampling time point. BAX 499 level was up to ten fold above the full length TFPI concentration.

### Example 3

### Properties of the BAX-TFPI Complex Formation

Immunoprecipitation studies and biomolecular interaction analysis were performed to identify the physiologic TFPI target for BAX 499 interaction and to characterize the kinetics of binding.

### Immuno-Precipitation of Plasma TFPI by Anti-TFPI Antibodies

TFPI antibodies were immobilized on an agarose resin with a Pierce® Direct IP Kit (Thermo Scientific) according to the manufacturer's instructions. Briefly, monoclonal anti-TFPI-KD2 antibody (Sanquin, White Label, MW1845) or monoclonal anti-TFPI-C-terminal antibody (Sanquin, White Label, MW1848), 40 μg each, were coupled to 100 μL of the amine reactive AminoLink Plus Coupling Resin included in the Kit. After quenching of residual active amine groups the resin was ready to use for immunoprecipitation.

For immune-precipitation of TFPI isoforms human pooled normal plasma (George King) or human TFPI depleted plasma (American Diagnostica) which served as a negative control, 2 mL each, were supplemented with 60 μg/mL recombinant hirudin (Hyphen Biomed) and 62 μg/mL corn trypsin inhibitor (Haematologic Technologies, Inc) for prevention of plasma clotting in the course of the experiment. Subsequently, 50 μL of antibody resins were added to the plasma samples and incubated for 1.5 hours, followed by resin centrifugation (2 min at 1000 g) and five wash cycles with 1 mL of 20 mM HEPES pH 7.4, 200 mM NaCl, 0.1% Tween20 (washing buffer). Finally, 60 μL of washing buffer and 40 μL 5× non-reducing SDS-PAGE sample buffer (Thermo Scientific) were added for disruption of protein-antibody complexes (final volume, 150 μL).

For Western blot analysis of standard proteins, including recombinant human fl-TFPI (Baxter Innovations GmbH) and recombinant human TFPI 1-160 (Baxter Innovations GmbH), and samples generated by immuno-precipitation experiments were loaded on a 12% SDS Tris-Glycine polyacrylamide gel (Mini-Protean TGX, BioRad) and blotted on a PVDF membrane (PVDF Mini Trans-Blot Transfer stack, BioRad). TFPI proteins were stained with a polyclonal rabbit anti-human TFPI antibody (ADG-72, American Diagnostica), followed by a polyclonal donkey anti-rabbit IgG HRP (NA9340, GE-Healthcare) antibody. Signals were developed with Super Signal West Femto Maximum Sensitivity Substrate (Thermo Scientific) and luminescence signal was imaged with a Fujifilm LAS-4000 device.

### Immuno-Precipitation of Plasma TFPI by BAX 499

For immobilization of biotinylated aptamer (ARC28635) 100 μL of pre-blocked high performance streptavidin sepharose (GE Healthcare) was incubated with 620 μL of ARC28635 (0.1 mM, BAXTER Innovations GmbH) for 1 hour, followed by 5 wash cycles. For immuno-precipitation of human pooled normal plasma (George King) or human TFPI depleted plasma (American Diagnostica) which served as a negative control, 2 mL each, were supplemented with 60 μg/mL recombinant hirudin (Hyphen Biomed) and 62 μg/mL corn trypsin inhibitor (Haematologic Technologies, Inc) for prevention of plasma clotting in the course of the experiment. Subsequently, 50 μL of antibody resins were added to the plasma samples and incubated for 1.5 hours, followed by resin centrifugation (2 min at 1000 g) and five wash cycles with 1 mL of 20 mM HEPES pH 7.4, 200 mM NaCl, 0.1% Tween20 (washing buffer). Finally, 60 μL of washing buffer and 40 μL 5× non reducing SDS-PAGE sample buffer (Thermo Scientific) were added for disruption of protein-aptamer complexes (final volume, 150 μL).

Western Blot analysis was performed as described above by using a monoclonal mouse anti-human TFPI antibody (Sanquin, White Label, MW1845) and a polyclonal sheep anti-mouse IgG HRP (A6782, Sigma) for TFPI detection.

For total protein staining of samples from the aptamer immuno-precipitation experiments proteins were separated on a 12% SDS Tris-Glycine polyacryl amid gel (Mini-Protean TGX, BioRad) and stained with the SilverXpress Kit (Invitrogen) according to the manufacturer's instructions.

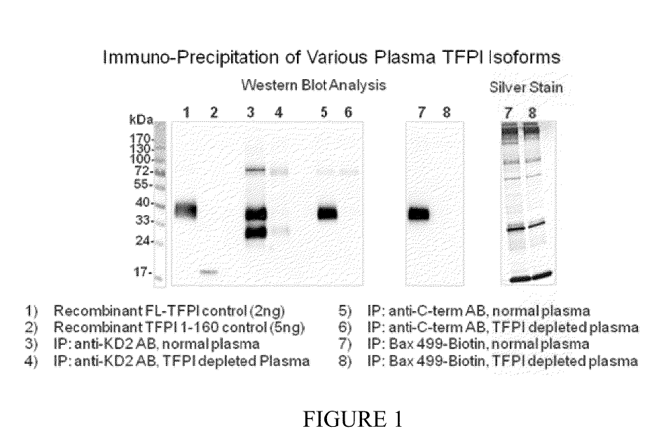

A monoclonal antibody which binds an epitope within Kunitz domain 2 of TFPI precipitated several plasmatic TFPI isoforms (FIG. 1, lane 3), one at about 80 kD which is possibly a dimeric TFPI, full length TFPI at about 40 kD and truncated TFPI at about 30 kD. The TFPI at about 30 kD is C-terminally truncated since it was not precipitated by an antibody which binds to the C-terminus of TFPI (FIG. 1, lane 5). Anti TFPI aptamer exclusively precipitated fl-TFPI (FIG. 1, lane 7). Total protein staining (silver staining) of these samples demonstrated minor non-specific binding to plasma proteins (FIG. 1, silver stain, lane 7 and 8). These experiments demonstrated that BAX 499 in a plasma milieu specifically binds to full length TFPI.

Kinetics of fl-TFPI-BAX 499 Interaction Studied by Biomolecular Interaction Analysis (BiaCore)

BAX 499 was further characterized for binding to fl-TFPI. Multiple experimental modifications were introduced. A biotinylated aptamer (ARC28635) with a nucleotide sequence identical to BAX 499 was used as a surrogate for binding of BAX 499 to fl-TFPI.

The binding kinetics of fl-TFPI (Baxter Innovations GmbH) to ARC28635 were studied using a BIAcore T200™ surface plasmon resonance assay (GE Healthcare, Chalfont St. Giles, UK) at 37° C. Following NeutrAvidin immobilization to a C1 chip (GE Healthcare) using standard amine coupling chemistry according to manufacturer's protocols ARC28635 was bound to the surface via biotin-NeutrAvidin interactions resulting in 75 RUs. Following aptamer immobilization fl-TFPI was injected for 100 s at a flow rate of 30 μL/min at concentrations ranging from 0.05 to 0.4 nM in HBS buffer pH 7.4, 0.1% P20, 3 mM CaCl₂. Subsequently, fl-TFPI was dissociated for 600 s. Biacore T200 Evaluation Software (GE Healthcare) was used to analyze the data. Association and dissociation parts of the sensorgrams were fitted separately according to a 1:1 Langmuir binding model. Means of kinetic parameters (kₐ, k_{d}) from individual concentrations were calculated and were used to calculate the binding constant (KD, kd/ka).

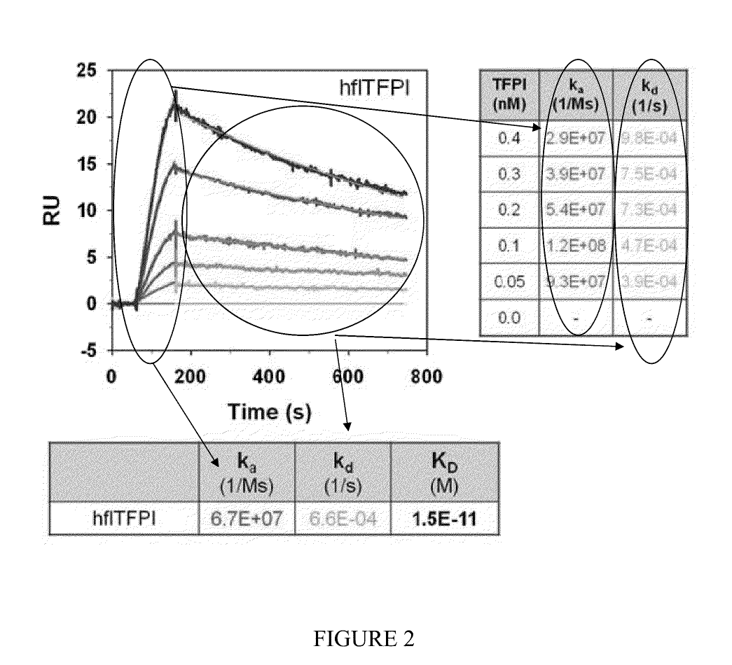

FIG. 2 demonstrates the binding of fl-TFPI to immobilized biotinylated aptamer (ARC28635) at concentrations ranging from 0 to 0.4 nM fl-TFPI (from bottom to top). Fitted data of association (kₐ) and dissociation (k_{d}) are indicated. Fl-TFPI bound tightly to the nucleic acid sequence of BAX 499 resulting in a binding constant of 15 pM. The association kinetics with a kₐ of 6.7×10⁷ 1/Ms was relatively fast, whereas the dissociation kinetics with k_{d} of 6.6×10⁻⁴ l/s was moderately slow.

### Example 4

### Characterization of the Distribution and Activity of Cellular TFPI and the Influences of BAX 499

TFPI is constitutively expressed by endothelial cells and directed to the intravascular lumen. A substantial fraction of TFPI remains at the cell surface. At least two isoforms of TFPI are described to be expressed by endothelial cell lines, TFPIα or fl-TFPI and TFPIβ. TFPIβ is covalently bound to cell surfaces via a GPI anchor whereas TFPIα is surface-associated by non-covalent interaction with GPI anchored proteins or by unspecific interaction with negatively charged glycosaminoglycans. A negatively charged compound like BAX 499 could release TFPI from endothelial cells by 1) competing with glycosaminoglycans for cell associated TFPI, 2) induction of TFPI expression and 3) mobilization from intra-cellular storage pools. In this example human umbilical vein endothelial cells (HUVECs) were used as a model to study the impact of BAX 499 on endothelial TFPI.

### Cell Culture

HUVECs were purchased from PromoCell (Heidelberg, Germany) and maintained in endothelial cell complete growth medium without antibiotics at 37° C. in a humidified incubator (5% CO2). The medium was composed of endothelial cell basal medium with endothelial cell growth supplement pack (both obtained from PromoCell) with a final media composition of 2% fetal calf serum (FCS); 0.4% endothelial cell growth supplement; 0.1 ng/mL epidermal growth factor (recombinant human) and 1 μg/mL hydrocortisone. Cells were detached with a mixture of trypsin:PBS (1:1) for 3 min at room temperature and reaction was stopped by adding trypsin neutralization solution (TNS, PromoCell). The cell suspension obtained was centrifuged for 3 min at 1100 g at room temperature and the cells were resuspended in prewarmed media. Cells were split at a ratio of 1:2 to 1:3 to cell culture wells containing complete growth medium prewarmed to 37° C. Medium exchange was performed every 2-3 days and cell number was determined by a Casy cell counter (Schärfe). Cells used for any experiment were not older than 13 transfers.

### Preparation of Cell Supernatants and Lysates

Cell culture supernatants were removed, filtrated through a 0.2 μm filter (Sartorius) and concentrated ˜5× by Amicon Ultra 0.5 centrifugal filter devices (Millipore; 10 kD cut off). Cells were washed twice with HBSS (Invitrogen) and lysed by the addition of 375 μL lysis buffer (0.1 M Tris/HCl; 0.15M NaCl; 0.5% Triton X-100; pH 7.8; 60 mM n-octyl-β-D-glycopyranosid) to each well. For lysis, cells were frozen and thawed twice. Following centrifugation for 10 min at 13.000 g, the supernatants obtained were analyzed for TFPI by an Enzyme-linked Immunosorbent Assay (ELISA).

### TFPI Release Assay

1.5×10⁵ cells/well were split on a 6 well plate and incubated for approximately 16-18 hours at 37° C. in a humidified incubator, followed by 3 wash cycles with complete growth media and incubation with fresh prewarmed media containing indicated compounds. Cells were incubated for 0.5 hours to 24 hours and at indicated time points supernatants and cells were harvested. Cell supernatants were analyzed for TFPI by ELISA.

### TFPI Immunoassay (ELISA)

The concentrations of TFPI in cell lysates and supernatants were assayed by an ELISA; the TFPI concentrations were referred to the cell number (100,000 cells). EDTA was added to all samples and standards for disrupting the interaction between TFPI and BAX 499. In control experiments it was shown that a final concentration of 20 mM EDTA was sufficient to abolish interference of BAX499 with the antibody binding to TFPI. For total TFPI quantification, wells of a microtiter plate (Nunc Maxisorp) were coated with 1 μg/mL of a monoclonal anti human KD2 specific TFPI antibody (Sanquin, White label; MW1845) overnight at 4° C., followed by 3 wash cycles with TBS containing 0.1% Tween 20 (TBST). Wells were blocked for 1 hour at room temperature with TBS containing 2% of non-fat dry milk (BioRad). 100 μL of undiluted sample were applied to the wells and incubated for 2 hours at room temperature. After washing with TBST (5×) wells were incubated for 1 hour with 0.5 μg/mL of a polyclonal rabbit anti hTFPI antibody (ADG72; American Diagnostica), washed 5× with TBST and incubated further for 1 hour with 0.2 μg/mL of a goat anti rabbit HRP labeled antibody (A0545; Sigma). Color was developed by addition of 100 μL of substrate (SureBlue TMP, KLP) and stopping with 504 of 1 M HCl. Absorbance at 450 nm was measured with a microtiter plate reader (Thermo Appliskan Reader). The fl-TFPI ELISA was performed as described above with the modification of using a monoclonal anti human C-terminus specific TFPI antibody (Sanquin, White label; MW1848) as a capture antibody. Purified endogenous fl-TFPI, expressed by SKHep cells, was used as standard protein for quantification (Baxter Innovations GmbH).

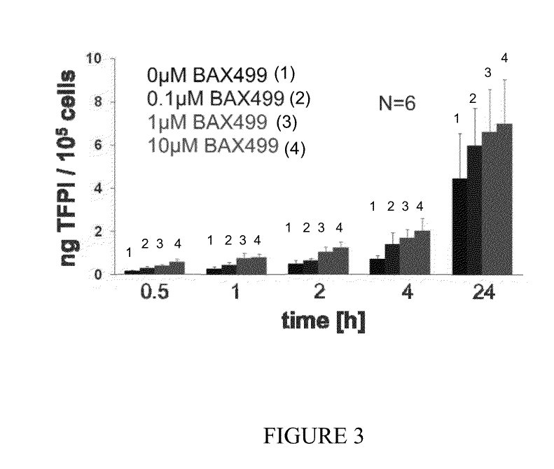

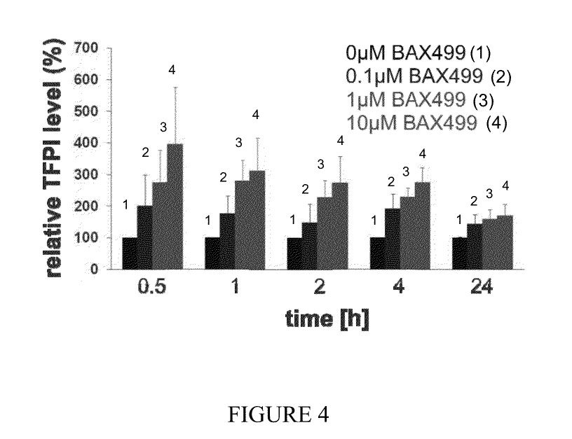

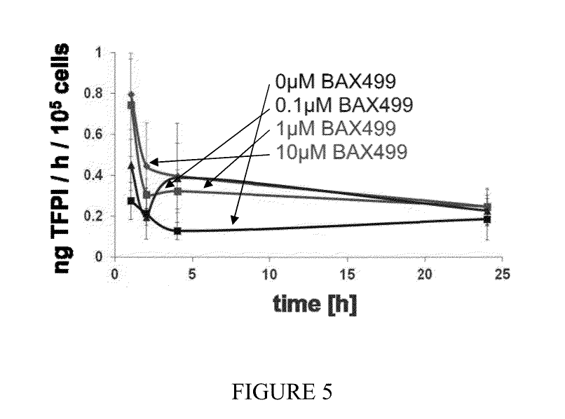

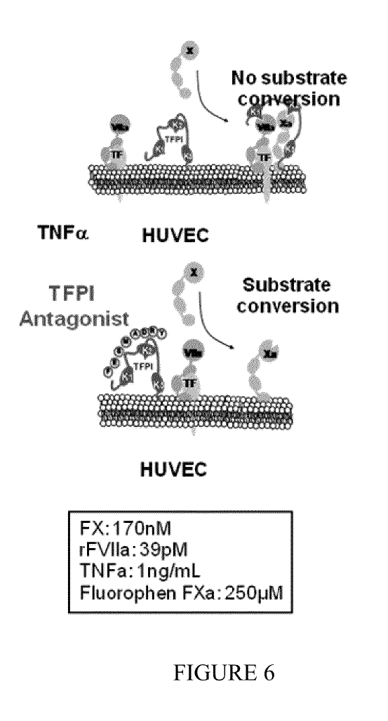

Release of cellular TFPI upon incubation with BAX 499 is illustrated in FIGS. 3-5. TFPI (total) concentrations (ng) per 105 HUVECs are indicated with means+/−standard deviations of 6 independent experiments (FIG. 3). TFPI levels relative to non-treated cells (labeled as “1”) are indicated with means+/−standard deviations of 6 independent experiments (FIG. 4). The rate of TFPI release (ng/h/105 cells) is indicated with means+/−standard deviations of 6 independent experiments (FIG. 5).

These experiments indicate that treatment of HUVECs with BAX 499 releases TFPI resulting in an up to 4-fold increase of TFPI in the media. The TFPI release was BAX 499 concentration dependent (FIGS. 3 and 4). The release of TFPI was most pronounced shortly after cells were incubated with BAX 499 as indicated by the increased rates of TFPI release at early time points (FIG. 5).

### Cell-Based FX Activation Assay

The functional inhibition of cell surface TFPI of BAX 499 was analyzed by a cell-based FX activation assay. HUVECs were split into wells of a 96 well plate (black flat with clear bottom; Corning) in complete growth medium at a density of 1.5×10⁴ cells per well. Cells were grown overnight (for approximately 16 to 18 hours), followed by two wash cycles with pre-warmed basal medium. Cells were then stimulated for tissue factor (TF) expression by the addition of recombinant tumor necrosis factor alpha (TNFα; Sigma) at a final concentration of 1 ng/mL to basal medium at 37° C. After 4 hours of incubation with TNFα the cells were washed twice with 200 μL of pre-warmed cell culture buffer (10 mM HEPES, 150 mM NaCl, 4 mM KCl, 11 mM Glucose, 5 mM CaCl₂; pH 7.5; 5 mg/mL BSA). Cells were further incubated for 20 min with 50 μL of cell culture buffer containing BAX 499 at increasing concentrations, coagulation factor VIIa (FVIIa) (Enzyme Research Laboratories) to allow complex formation. A polyclonal anti-human TFPI antibody (R&D Systems, AF2974) (100 nM final concentration) served as a positive control for TFPI inhibition. Factor X activation was initiated by the addition of 50 μL of pre-warmed cell culture buffer, containing factor X (FX) and factor Xa specific substrate (Fluophen FXa, HYPHEN). Final assay concentrations were: 39 pM FVIIa; 170 nM FX, 250 μM Fluophen FXa and BAX 499 as indicated in 100 μL final volume. The 96 well plate was transferred to a pre-warmed fluorescence reader (TECAN; Safire2) and fluorescence signal (excitation 360 nM, emission 440 nm), resulting from the cleavage of the FXa substrate, was measured for 20 min at 37° C. Fluorescence was converted to FXa [nM] according to the formula: Xa=d/dt AMC*(Km+S0−AMC)/(kcat*(S0−AMC))*1000. FXa concentrations obtained at 9 min of incubation were used for calculation of TFPI inhibitors effects. FXa levels of reactions with polyclonal anti human TFPI antibody served as 100% TFPI inhibition reference, FXa levels obtained in the absence of any compound was considered as 0% TFPI inhibitory effect.

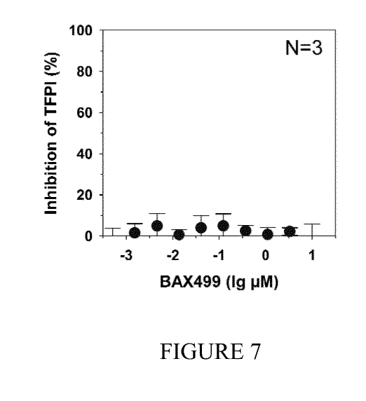

The inhibition of cell surface TFPI activity expressed in relation to a polyclonal anti TFPI antibody that fully inhibits cell surface TFPI (100% inhibition) is shown in FIG. 7. With means+/−standard deviations from three independently performed experiments indicated, the HUVE cell-based factor Xa activation experiment indicates that BAX 499 up to concentrations of 10 μM did not interfere with cell surface TFPI activity (FIG. 7).

Cell Based FX Activation Assay with BAX 499 Treatment Prior to Cell Surface TFPI Activity Assessment

For characterization of TFPI activity released by BAX 499 from cell surfaces an assay quite similar to that described in the previous section was performed. BAX 499 was incubated simultaneously with TNFα for TF stimulation prior to FX activation. This allows one to estimate the total TFPI activity on the cell surface. Cell were washed to ensure the complete removal of BAX 499, FX activation was assessed as described above.

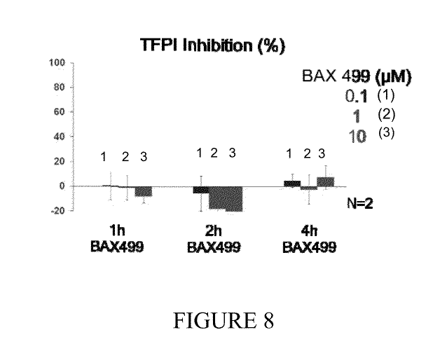

FIG. 8 illustrates the impact of BAX 499 on total cell surface TFPI activity in a HUVE cell-based FX assay. Decrease of cell surface TFPI activity is expressed in relation to a polyclonal anti TFPI antibody which fully inhibits cell surface TFPI (100% inhibition). Means+/−standard deviations from two to three independently performed experiments are indicated. HUVECs incubated up to 4 hours with up to 10 μM of BAX 499 did not demonstrate reduced TFPI activity as assessed by a cell-based FX activation assay (FIG. 8). This indicates that BAX 499 does not remove TFPI activity from the surface of HUVECs.

TFPI mRNA Quantification by Real Time Polymerase Chain Reaction

To investigate if increased TFPI is caused by an induction of TFPI expression, TFPI mRNA was quantified by real time PCR. Cells obtained from release assays (see above) were lysed and total RNA was isolated using Tri Reagent (Sigma) according to the supplier's protocol. cDNA was generated by reverse transcription of total RNA (1 μg) using QuantiTect Reverse Transcription Kit (Qiagen). For TFPI alpha gene product quantification the following forward and reverse primers were used: 5′-AGC TCA ATG CTG TGA ATA ACT CC-3′ and 5′-TTG GCA CGA CAC AAT CCT CTG-3′. For actinβ the following forward and reverse primers were used: 5′-GAT GAT GAT ATC GCC GCG CTC-3′ and 5′-CCA CAT AGG AAT CCT TCT GAC C-3′. TFPI and actinβ real time PCRs were performed in separate tubes with 0.83 μL reverse transcription products in a total volume of 25 μL. Real time PCRs were performed using QuantiTect SYBR Green PCR Kit (Qiagen) on an Applied Biosystems 7300 Real-Time PCR System. Calculations were performed with the 7300 System SDS software version 1.4. Results were normalized to the expression of actinβ as a housekeeping gene product. Results are expressed in relation to the untreated control which was set to 100%.

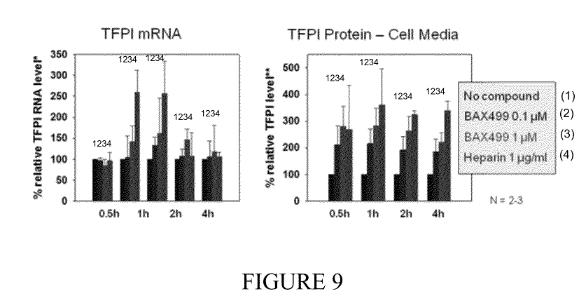

FIG. 9 illustrates the impact of BAX 499 on TFPI gene expression as quantified by real time PCR. TFPI mRNA levels relative to non-treated cells are indicated. Means+/−standard deviations from two to three independently performed experiments are indicated. In contrast to heparin which is known to induce TFPI mRNA expression, BAX 499 up to 1 μM had no effect on TFPI synthesis at the DNA/RNA level (FIG. 9).

### Flow Cytometry

9×10⁵ HUVE cells were split on a 10 cm plate and incubated for approximately 16-18 hours at 37° C. in a humidified incubator. Cells were washed 3 times with complete growth media and incubated with fresh pre-warmed media containing the indicated compound. After incubation for 30 min or 120 min the cell culture supernatants were harvested, snap-frozen on dry ice and analyzed later for their TFPI levels. Cells were washed three times with HBSS and then detached by enzyme-free cell dissociation solution (Millipore) to preserve the structural and functional integrity of cell surface proteins. Following two wash cycles with PBS, one third of the cells were kept intact for cell surface TFPI analysis. Cells were blocked by incubation with ice-cold blocking buffer (10% FCS in PBS) for 30 mins at 4° C. The remaining cells were fixed for 30 min (BD Cytofix) at 4° C. for intracellular and cell surface TFPI (total cellular TFPI) analysis. Cells were passed through a cell strainer cap (BD) and were finally washed and permeabilized by blocking buffer containing 0.1% triton X-100. Living, non-permeabilized and fixed, permeabilized cells were stained with a rabbit polyclonal anti human TFPI antibody (ADG72, American Diagnostica, 50 μg/mL) in blocking buffer for 45 min at 4° C. After three wash cycles with blocking buffer cells were incubated with Alexa Fluor 633 goat anti-rabbit IgG (Molecular Probes) in the same buffer for 30 min in the dark on ice. For living cells 7.5 μg/mL and for fixed cells 0.6 μg/mL of this fluorophor-labelled antibody was used. Cells incubated with isotype control (rabbit XP, Cell Signalling) or with the secondary antibody alone served as negative controls. Stained cells were analyzed by fluorescence activated cell sorting flow cytometry (BD FACSCanto II). Signals from at least 1×10⁴ cells were analyzed by using FlowJo software version 7.2.2 (TreeStar). Results were expressed as mean relative fluorescence intensity (% MFI). Results obtained from untreated cells, were set to 100% and results from incubations with test items are given in relation to these.

FIG. 10 illustrates cell surface TFPI by Fluorescence-activated cell sorting (FACS) analysis of non-permeabilized cells and cell supernatant TFPI by ELISA. Mean relative fluorescence intensity (%) in relation to non-treated cells (labeled as “1”) and cells treated with BAX 499 (0.1 μM, labeled as “2”), BAX 499 (1 μM, labeled as “3”), heparin (1 μg/mL, labeled as “4”) and phosphatidylinositol phospholipase C (0.1 U/mL, labeled as “5”) is indicated on left hand y-axis and is represented as full bars. TFPI levels in supernatants from cultured cells in relation to non-treated cells are indicated on the right hand y-axis (% TFPI level) and are represented as open bars. Experimental error is indicated by standard deviations from means of 3 independently performed experiments (FIG. 10).

FIG. 11 illustrates total cellular TFPI by FACS analysis of fixed, permeabilized cells and cell supernatant TFPI by ELISA. Mean relative fluorescence intensity (%) in relation to non-treated cells (labeled as “1”) and cells treated with BAX 499 (0.1 μM, labeled as “2”), BAX 499 (1 μM, labeled as “3”), heparin (1 μg/mL, labeled as “4”) and phosphatidylinositol phospholipase C (0.1 U/mL, labeled as “5”) is indicated on left hand y-axis and is given as full bars. TFPI levels in supernatants from cultured cells in relation to non treated cells is indicated on right hand y-axis (% TFPI level) and is given as open bars. Experimental error is given by standard deviations from means of 3 independently performed experiments (FIG. 11).

FACS analysis of non-permeabilized HUVECs, which is a measure of cell surface TFPI indicated that BAX 499 and heparin treatment do not impact cell surface TFPI, whereas PIPLC treatment results in an about 80% reduction of TFPI located on the surface of HUVECs (FIG. 10). Assessment of total cellular (surface and intracellular) TFPI by FACS analysis of fixed and permeabilized HUVECs demonstrated a reduction of TFPI by about 20% when cells were treated with BAX 499, heparin and PIPLC (FIG. 11).

### Example 5

### Impact of BAX 499 on fl-TFPI Clearance

Receptor-mediated clearance of TFPI regulates its plasma concentration and low density lipoprotein receptor-related protein (LRP) mediates rapid clearance and cellular degradation of TFPI after TFPI binding to the hepatoma cell surface. The carboxy-terminal regions of TFPI were described to mediate binding to hepatoma cells in vitro and in vivo. This example investigates the impact of BAX 499 on the clearance of fl-TFPI.

Impact of BAX 499 on fl-TFPI Low Density Lipoprotein Receptor-Related Protein (LRP) Interaction Studied by BiaCore

The binding fl-TFPI (Baxter Innovations GmbH) to LRP was studied using a BIAcore T200™ surface plasmon resonance assay (GE Healthcare, Chalfont St. Giles, UK) at 37° C. Following Neutravidin immobilization (2500 RU) to a Series S Sensor chip C1 (GE Healthcare) using standard amine coupling chemistry according to manufacturer's protocols, biotinylated recombinant human LRP-1 Cluster II Fc Chimera protein (R&D Systems) was bound to the surface via biotin—NeutrAvidin interactions resulting in 450 RUs. Prior to immobilization LRP was biotinylated using a biotinylation kit according to the manufacturer's protocol (Thermo Scientific). Following LRP immobilization fl-TFPI was injected by a single cycle application mode at a flow rate of 30 μL/min at concentrations ranging from 3.6 to 142.3 nM diluted in running buffer (HBS-N, 0.1% P80, 5 mM CaCl₂). Subsequently, fl-TFPI was dissociated by changing the flow to running buffer conditions. When interaction of fl-TFPI and LRP was studied in the presence of BAX 499, 1 μM final concentration of BAX 499 was added to each of the fl-TFPI concentrations.

Binding of fl-TFPI to immobilized biotinylated LRP at concentrations ranging from 0 to 142 nM fl-TFPI in the absence and presence of BAX 499 (1 μM) is shown in FIG. 12. Fl-TFPI interacted efficiently with immobilized LRP (FIG. 12) with fast on- and off-rates. Due to a presumably complex mode of interaction it was not possible to fit the data to any binding model provided by the software. By visual inspection it seems that fl-TFPI when bound to BAX 499 interacts less efficiently with LRP (FIG. 12). In contrast to fl-TFPI alone, fl-TFPI complexed to BAX 499 marginally bound to LRP at low concentrations. Binding of fl-TFPI-BAX 499 complex was observed at higher concentrations (>23 nM) with seemingly changed binding kinetics. This demonstrates that BAX 499 interfered with the binding of fl-TFPI to LRP, a receptor which has been described to be involved in the clearance of TFPI.

Impact of fl-TFPI-BAX 499 Complex Formation on the Pharmacokinetics of fl-TFPI Studied in Mice

To study a possible impact of BAX 499 on the pharmacokinetics of human fl-TFPI in vivo a mouse study was performed. Groups of mice (C57B16, male, 20-25 g) were treated with either human fl-TFPI (775 nM, 5 mL/kg i.v.), human fl-TFPI complexed to a 10-fold molar excess of ARC17480 (775 nM hu fl-TFPI, 775 nM ARC17480, 5 mL/kg, i.v.) or human fl-TFPI complexed to a 10-fold molar excess of BAX 499 (775 nM hu fl-TFPI, 7752 nM BAX 499, 5 mL/kg, i.v.). ARC 17480 has the same nucleic acid sequence as BAX 499, lacks however the PEG-modification and serves as a control for a possible impact of compound PEGylation. At each time point three mice were sacrificed, blood was taken by heart puncture, plasma was generated and stored frozen (<−60° C.) for further analysis. The sampling time points for each test item were as follows: fl-TFPI, 0.5, 1, 2 min; fl-TFPI-ARC17480, 0.5, 1, 2, 3, 5, 8 min and fl-TFPI-BAX 499, 1, 2, 5, 10, 20, 35 min. Plasma samples were analyzed for human TFPI with an ELISA according to Example 4 which is specific for human TFPI. Plasma samples were diluted from 1/20 to 1/800 depending on the expected human TFPI concentration.

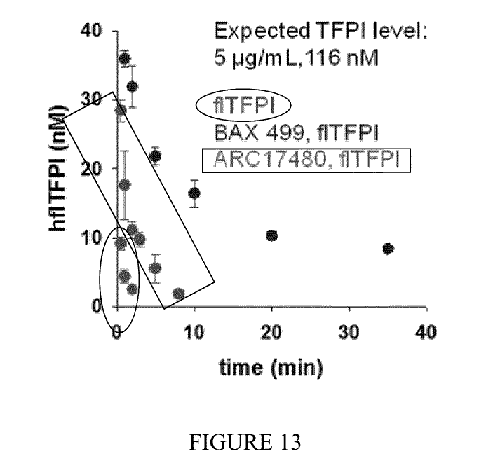

FIG. 13 demonstrates the pharmacokinetics of human fl-TFPI in mice. Human fl-TFPI were administered alone (circled), bound a 10-fold molar excess of ARC 17480 (squared) or bound to a 10-fold molar excess of BAX 499 (unmarked). Human fl-TFPI concentrations (nM) are indicated as means+/−standard deviation from three animals at each time point. Human fl-TFPI has a very short half-life and a very poor in vivo recovery. At the earliest time point (0.5 min) only one tenth of the expected TFPI level was observed (FIG. 13, circled). When fl-TFPI was bound to ARC17480 about 30 nM of TFPI was observed at the earliest time point which is indicative of an improved recovery. BAX 499 improved the recovery and substantially prolonged the half-life of human fl-TFPI. This experiment indicates that BAX 499 imposed its long half-life to fl-TFPI and interfered with the clearance of TFPI.

### Example 6

### BAX 499 Delays Proteolytic Degradation of fl-TFPI

TFPI is proteolytically inactivated by several enzymes. The region between Lys86 and Gln90 located between the Kunitz 1 and Kunitz 2 domain of TFPI has been described as a hot spot region, as it contains cleavage sites for several proteases including elastase, thrombin, plasmin, FXa, elastase and chymase (Hamuro, FEBS 2007). The impact of BAX 499 on the proteolytic degradation of fl-TFPI by neutrophil elastase and plasmin, representative proteases cleaving within the hot spot as well as other regions, were studied. Proteolysis of TFPI was monitored by Western blot analysis and by an activity assay that measured the ability of TFPI to inhibit factor Xa (FXa).

For the proteolytic digest of fl-TFPI protein (Baxter Innovations GmbH), 5 nM fl-TFPI were incubated with 5 nM neutrophil elastase (purified from human neutrophils, Calbiochem) or 50 nM plasmin (human lys-plasmin purified from plasma, American Diagnostica, Inc.) in a reaction buffer (50 mM Tris, 300 mM NaCl, 10 mM CaCl₂ pH 7.5) at 37° C. The reaction was performed in the presence of 0, 0.1 and 1 μM BAX 499. For Western blot analysis, aliquots were taken from the reaction mixture after 0, 5, 15, 30, and 60 min and immediately heated at 96° C. for 5 min in SDS-loading buffer under reducing conditions. Samples were separated on a 4-20% Tris-glycine SDS-polyacrylamide gel and the proteins transferred to a PVDF membrane. After blocking of the membrane with non-fat dry milk, TFPI proteins were detected with a rabbit polyclonal antibody against human TFPI (AF2974, R&D Systems) and a secondary anti-rabbit-HRP conjugated antibody (Sigma). SuperSignal West Femto chemiluminescent substrate (Thermo Scientic) was applied to develop a chemiluminescent signal that was captured on film. The developed bands were quantified by densitometry.

For assessment of TFPI residual activity after incubation with elastase/BAX 499, FXa inhibition by TFPI was determined via an FXa inhibition assay. At the same time points as for Western blot analysis, aliquots were removed from the proteolysis reaction and 50 nM antitrypsin (Sigma) was added to block any elastase activity. TFPI inhibitory activity was determined in a reaction buffer (25 mM HEPES, 175 mM NaCl, 5 mM CaCl₂, 0.1% BSA, 20 mM EDTA, pH 7.35) containing final concentrations of 0.1 nM FXa (Enzyme Research Laboratories) 10 μM phospholipids (80% phosphatidylcholine/20% phosphatidylserine) and 0.4 mM S-2222 chromogenic substrate (Chromogenix) to follow FXa activity in time. Final assay concentrations of TFPI or degradation products thereof were 0.43 nM. Standard intact fl-TFPI protein (0.1-0.43 nM) was used as a control. Chromogenic activity was detected at 405 nm at 37° C. for 2 h. After 115 min, the maximal inhibition of FXa by TFPI was determined for each sample. The inhibitory effect of TFPI in each sample was expressed as % activity of the fl-TFPI at the 0 min time point of incubation with neutrophil elastase (=100%).

FIG. 14 illustrates a time course of TFPI digestion by human neutrophil elastase in the absence and presence of BAX 499 (1 μM); and the FXa inhibitory activity of TFPI. In the absence of BAX 499, gradual degradation of fl-TFPI by neutrophil elastase was observed within 1 hour (FIG. 14A). The ˜43 kD band of the intact fl-TFPI almost completely disappeared while two cleavage products were formed upon cleavage of the peptide bond T87-T88 by neutrophil elastase. When BAX 499 (1 μM) was present in the reaction mixture, the proteolysis of fl-TFPI occurred slower over time, which indicates that BAX 499 protected fl-TFPI from degradation by elastase. The results of the TFPI-activity assay further supported these findings. Without BAX499, the inhibitory activity of fl-TFPI towards FXa was diminished to ˜34% upon degradation by elastase for 30 min (FIG. 14B). In the presence of 0.1 and 1 μM BAX 499 remaining activity was ˜46% and ˜65%, respectively. Proteolytic degradation of fl-TFPI by plasmin was also delayed within the first 30 minutes in the presence of 1 μM BAX 499 (data not shown).

The data demonstrate that BAX 499 delays proteolytic degradation of fl-TFPI by human neutrophil elastase and plasmin in vitro. Therefore, BAX 499 can influence the metabolism of TFPI in vivo by protecting it from proteolytic attack and can consequently contribute to half-life extension of TFPI in circulation.

### Example 7

### Inhibition of TFPI by BAX 499 is Diminished as TFPI Levels Increase

To understand the consequences of elevated plasma TFPI, the TFPI-inhibitory activity of BAX 499 was tested at increasing TFPI concentrations using a FXa inhibition and an extrinsic tenase inhibition assay. These assays may be predictive for BAX 499 activity in plasma. The extrinsic tenase assay gives insight into the influence of the TFPI antagonists on (a) the interaction of FXa and TFPI and (b) the interaction of the FXa-TFPI complex with the TF-FVIIa complex. The FXa inhibition assay measures a BAX 499 influence on the interaction of FXa and TFPI only.

The extrinsic tenase complex is responsible for FX and FIX activation upon initiation of the coagulation process. The extrinsic complex is composed of FVIIa, Tissue Factor (TF), and FX substrate. To determine the influence of TFPI antagonists on the TFPI-mediated inhibition of the extrinsic tenase complex, a coupled enzyme assay was established. BAX 499 was diluted in 1.25× reaction buffer+0.1% Tween-80 (31.25 mM HEPES/218.75 mM NaCl/6.25 mM CaCl₂/0.125% BSA; pH 7.35). TFPI, FVIIa, and lipidated TF were diluted in 1.25× reaction buffer. Phospholipid vesicles (DOPC/POPS 80/20), and chromogenic substrate specific for FXa (S-2222 (available from DiaPharma, West Chester, Ohio)), diluted in aqua dest were added to 96-well plates. After an incubation period, fl-TFPI and BAX 499 dilutions were added. FX activation was initiated by adding FX to the wells. FXa-mediated chromogenic substrate conversion was determined by observing an increase in absorbance using a micro-plate reader. The amount of FXa generated at certain time points was calculated from the OD readings. FXa generated at 20 min after start of the reaction was considered for calculation of the EC50 from plots of BAX 499 concentration versus the inhibition of TFPI (%).

The functional inhibition of TFPI also was examined using an FXa inhibition assay. A FXa-specific chromogenic substrate (5-2222) and phospholipid vesicles (DOPC/POPS 80/20), both diluted in aqua dest and TFPI protein (human fl-TFPI) diluted in 1.25× reaction buffer, were added to 96 well plates. BAX 499 was diluted in 1.25× reaction buffer+0.1% Tween-80. The BAX 499 dilutions (2.5 μL) were added to the 96 well plates. The conversion of chromogenic substrate was triggered by the addition of FXa, and the kinetics of the conversion was measured in a micro-plate reader. Because TFPI inhibits FXa slowly, OD readings after 115 minutes were considered for calculation of the EC50 from plots of BAX 499 concentration versus the inhibition of TFPI.

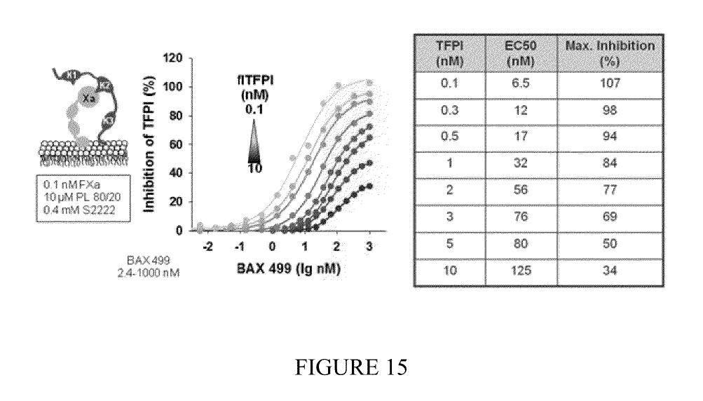

FIG. 15 illustrates TFPI inhibition by BAX 499 (2.4-1000 nM) at increasing TFPI levels (0.1-10 nM from left to right) in a FXa inhibition assay. Data points were fitted by a sigmoidal dose response equation resulting in EC50 values (nM) and maximum inhibition (%).

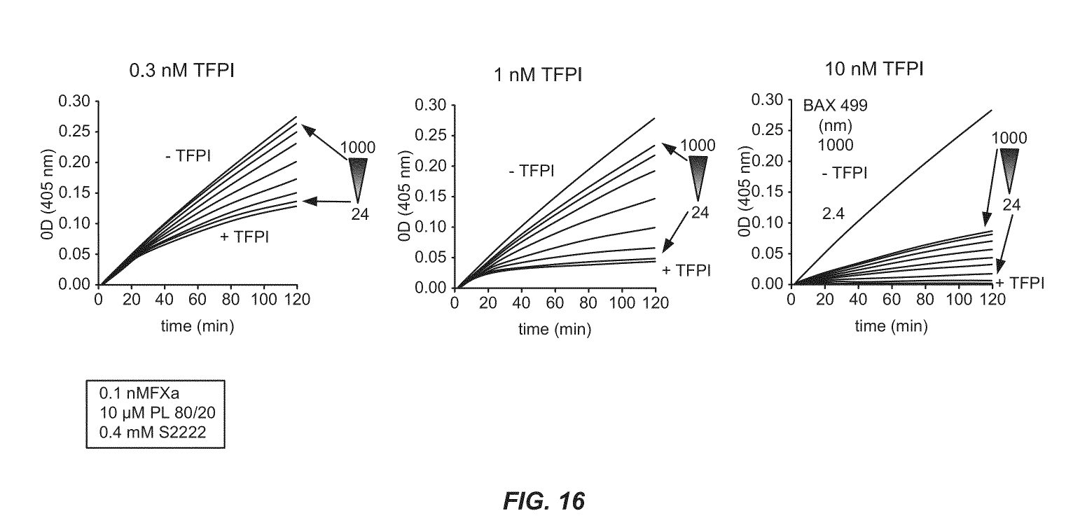

FIG. 16 shows the progression of FXa inhibition at three selected TFPI concentrations (0.3, 1 and 10 nM) and increasing BAX 499 (2.4 to 1000 nM). The top line is FXa activity in absence of TFPI (−TFPI), whereas the bottom line is FXa activity which is progressively inhibited by the indicated TFPI concentration in the absence of BAX 499.

BAX 499 efficiently inhibited fl-TFPI when tested at low (physiologic) fl-TFPI concentrations (<0.5 nM) in a FXa activity assay (FIG. 15). 1000 nM of BAX499 almost completely reversed the inhibition of FXa by TFPI. However, at increasing TFPI the inhibition of TFPI by BAX499 became much less efficient which was reflected by increasing EC50 values and decreasing maximum inhibition. At substantially increased TFPI concentrations (e.g. 10 nM) EC50 was about 20-fold increased and TFPI retained about 70% of its FXa inhibitory activity. This suggests that BAX 499 is a partial inhibitor of TFPI which efficiently neutralizes it at low (physiologic) concentrations but inefficiently inhibits at high TFPI levels.

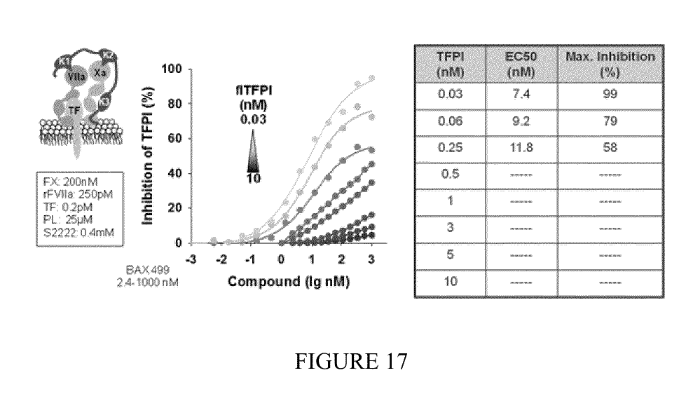

Similar observations were made in a more complex reaction system (TF-FVIIa-catalyzed FX activation) (FIG. 17). FIG. 17 shows TFPI inhibition by BAX 499 at increasing TFPI concentrations (0.03 to 10 nM from left to right) in an extrinsic tenase inhibition assay. Data points were fitted by a sigmoidal dose response equation resulting in EC50 values (nM) and maximal inhibition (%). In this system, very low TFPI concentrations (e.g. 0.03 nM) were efficiently inhibited by BAX 499. At physiologic TFPI concentrations (e.g. 0.25 nM) TFPI was inhibited with an EC50 of about 12 nM. At concentrations as high as 1000 nM BAX499 only partially (58%) reversed the inhibition TF-FVIIa-catalyzed FX activation by TFPI. 1000 nM BAX499 hardly reversed inhibition of TF-FVIIa-catalyzed FX activation by 10 nM TFPI, suggesting that elevated TFPI even when fully complexed to BAX 499 retained substantial inhibitory activity on TF-FVIIa-catalyzed FX activation.

### Example 8

### Inhibition of TFPI by BAX 499 is Diminished as TFPI Levels Increase as Determined by Plasma-Based Thrombin Generation Assay

In this example, the TFPI inhibitory activity of BAX 499 was explored using a plasma-based thrombin generation assay. To assess the TFPI inhibitory activity, BAX 499 was tested at physiological conditions (−0.2 nM fl-TFPI) as well as at elevated fl-TFPI (up to 10 nM fl-TFPI), similar to the conditions observed in plasma samples of hemophilia patients treated with BAX 499.

The influence of BAX 499 in the absence or in the presence of exogenous fl-TFPI on thrombin generation was measured in duplicate via calibrated automated thrombography in a Fluoroskan Ascent® reader (Thermo Labsystems, Helsinki, Finland; filters 390 nm excitation and 460 nm emission) following the slow cleavage of the thrombin-specific fluorogenic substrate Z-Gly-Gly-Arg-AMC (Hemker, Pathophysiol. Haemost. Thromb., 33, 4-15 (2003)). As a model for antibody-mediated FVIII deficiency, frozen pooled normal plasma (George King Bio-Medical Inc.,) was incubated with high titer, heat inactivated, anti-human FVIII plasma raised in goat (4490 BU/mL; Baxter Innovations GmbH) giving rise to 50 BU/mL. The plasma was mixed with corn trypsin inhibitor (CTI) (Hematologic Technologies, Inc., Essex Junction, Vt.) to inhibit Factor XIIa contamination, resulting in a final concentration of 40 μg/mL.

Pre-warmed (37° C.) plasma (80 μL) was added to each well of a 96 well micro-plate (Immulon 2HB, clear U-bottom; Thermo Electron). To trigger thrombin generation by tissue factor (“TF”), 10 μL of PPP low reagent containing low amounts (12 pM) of recombinant human TF and phospholipid vesicles composed of phosphatidylserine, phosphatidylcholine and phosphatidylethanolamine (48 pM) (Thrombinoscope BV) were added. Just prior to putting the plate into the reader, 5 μL BAX 499 dilutions were added, resulting in plasma concentrations of 10-1000 nM. Finally, 5 μL of the fl-TFPI dilutions or 5 μL HEPES buffered saline with 5 mg/mL bovine serum albumin (Sigma-Aldrich) were added. The fl-TFPI protein (3557 nM) had been expressed in SK Hep cells and purified. Plasma concentrations of fl-TFPI varied between 0.3 and 10 nM depending on the experiment, which was equivalent to a ˜2 to 50-fold increase in endogenous fl-TFPI plasma concentration. Thrombin generation was initiated by dispensing into each well 20 μL of FluCa reagent (Thrombinoscope BV, Maastricht, The Netherlands) containing a fluorogenic substrate and HEPES-buffered CaCl₂ (100 mM). Fluorescence intensity was recorded at 37° C.

The parameters of the resulting thrombin generation curves were calculated using Thrombinoscope™ software (Thrombinoscope BV, Maastricht, The Netherlands) and thrombin calibrator to correct for inner filter and substrate consumption effects (Hemker, Pathophysiol. Haemost. Thromb., 33, 4-15 (2003)).

FIG. 18 illustrates thrombin generation of FVIII inhibited plasma in presence of BAX 499 (1000 nM) at increasing fl-TFPI (up to 5 nM). Pooled normal plasma (indicated); Pooled normal plasma plus anti FVIII, FVIII-inhibited plasma (indicated); FVIII-inhibited plasma in presence of BAX 499 (1000 nM) at increasing exogenous fl-TFPI is indicated. Endogenous plasma TFPI was quantified by ELISA (fl-TFPI: 0.3 nM, total TFPI: 1.3 nM).

BAX 499 substantially increased thrombin generation of FVIII-inhibited plasma and corrected peak thrombin to normal conditions (FIG. 18) at physiological plasma TFPI concentrations. At increasing fl-TFPI (exogenously added) the pro-coagulant activity of BAX 499 was increasingly reversed as demonstrated by a reduction in peak thrombin and endogenous thrombin potential (area under the thrombin generation curve). The time parameters (lag and peak time) of thrombin generation were minimally affected by increasing fl-TFPI.

FIG. 19 illustrates inhibition of elevated plasma concentrations of human fl-TFPI by BAX 499 (10-1000 nM) in a thrombin generation assay in FVIII-inhibited plasma. Solid reference line indicates base-line peak thrombin (left) or ETP (right) of FVIII-inhibited plasma; dashed reference line indicates peak thrombin (left) or ETP (right) of pooled normal plasma. Endogenous plasma TFPI was quantified by ELISA (flTFPI: 0.3 nM, total TFPI: 1.3 nM).

FIG. 20 illustrates BAX 499 requirement for neutralization of elevated fl-TFPI. The line is an exponential fit of data points calculated based on peak thrombin values from FIG. 19. FIG. 20 demonstrates the TFPI-antagonistic potential of BAX 499 in the presence of a wide concentration range of flTFPI (up to 10 nM), which is equivalent to about 50-fold higher than physiological fl-TFPI plasma concentration. Generally, a substantial excess of BAX 499 was required for compensation of the anticoagulant activity of increasing fl-TFPI. Based on peak thrombin data (FIG. 20) at given BAX 499 concentrations fl-TFPI levels were calculated which could be neutralized to reach peak thrombin baseline level of FVIII-inhibited plasma. The requirement of BAX 499 to neutralize increasing fl-TFPI increased exponentially. A ˜50-fold excess of BAX 499 neutralized 0.2 nM TFPI added to FVIII-inhibited plasma whereas a 140-fold excess was required for neutralization of 7.3 nM additional fl-TFPI. A pair of BAX 499 and TFPI concentrations which would be under the fitted line (FIG. 20) would support procoagulant activity of BAX 499, whereas a pair above would result in a net anticoagulant effect.

Together the data in model systems and in plasma show that BAX499 even at a concentration of 1000 nM, which is more than 10,000 times above the Kd of the BAX499-TFPI complex, was not able to reverse and hardly affected the inhibition of FXa, TF-FVIIa-catalyzed FX activation in model systems and thrombin generation in plasma by 10 nM TFPI. The data also suggest a mechanism for increased bleeding problems in hemophilia patients associated with the more than 25-fold elevated TFPI plasma levels after BAX499 administration. These data suggest that BAX499 is not able to compensate the anticoagulant activity of the increased TFPI levels in the patients treated with high doses of BAX499.

### Example 9

### Clot Formation in Whole Blood

This example describes a method for assessing clot formation in whole blood using rotation thromboelastometry (ROTEM®). Rotational Thromboelastometry (ROTEM®) is a continuous visco-elastic assessment of whole blood clotting. Recalcification of citrated whole blood and low concentrations of lipidized TF are used to initiate clot formation. ROTEM® tracings show elasticity (mm) versus time (s). ROTEM® parameters were recorded according to the manufacture's manual and include the clot time (CT). CT is defined as the time from the start of measurement to the start of clot formation (amplitude of 5 mm). The clot formation time (CFT) is defined from the amplitude of 5 mm until an amplitude of 20 mm is reached. The alpha angle displays the formation of the fibrin clot and MCF is the maximum difference in amplitude between the two traces achieved during the assay.

Human whole blood clot formation and firmness was performed by ROTEM with whole blood preparations in the presence or absence of BAX 499. Blood samples from a healthy individual were drawn into citrated Sarstedt S-Monovette (0.106 M or 3.2% (w/v) Na-citrate) (5 mL), mixing one part of citrate with nine parts blood, using a 20 gauge needle. A portion of the blood samples was incubated with high titer, heat-inactivated anti-human FVIII antiplasma raised in goat (4488 BU/mL; Baxter Innovations GmbH) resulting in 51 BU/mL. Test samples were prepared by dissolving quantities of BAX 499 in HEPES buffered saline with 5 mg/mL BSA (Sigma Aldrich). Recordings were made using a ROTEM thromboelastometry coagulation analyzer (Pentapharm, Munich, Germany) at 37° C. Before starting measurement, the citrated whole blood was mixed with corn trypsin inhibitor (CTI) (Hematologic Technologies, Inc., Essex Junction, Vt., USA) providing a final concentration of 62 μg/mL. CTI specifically inhibits FXII and thus prevents FXIIa-mediated contact activation. The analytical set-up was as follows: to a BAX 499 sample (10, 100, 1000 nM final blood concentration) or a control, 300 μL of pre-warmed (37° C.) CTI treated citrated whole blood was added, followed by addition of a 1:17 dilution of TF PRP reagent containing recombinant human TF (rTF, 12 pM) (TS40, Thrombinoscope BV). In certain experiments, exogenous fl-TFPI at blood concentrations of 2 or 10 nM was added to simulate fl-TFPI levels of up to 50-fold over normal. Coagulation was initiated by the addition of 20 μL 200 mM CaCl₂ (star-TEM®, Pentapharm, Munich, Germany) and recordings were allowed to proceed for at least 120 min. The final concentration of rTF in the assay was 44 fM.

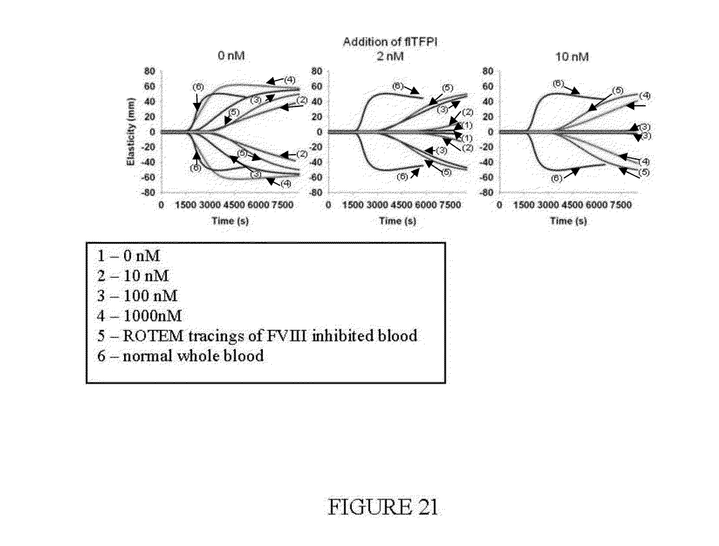

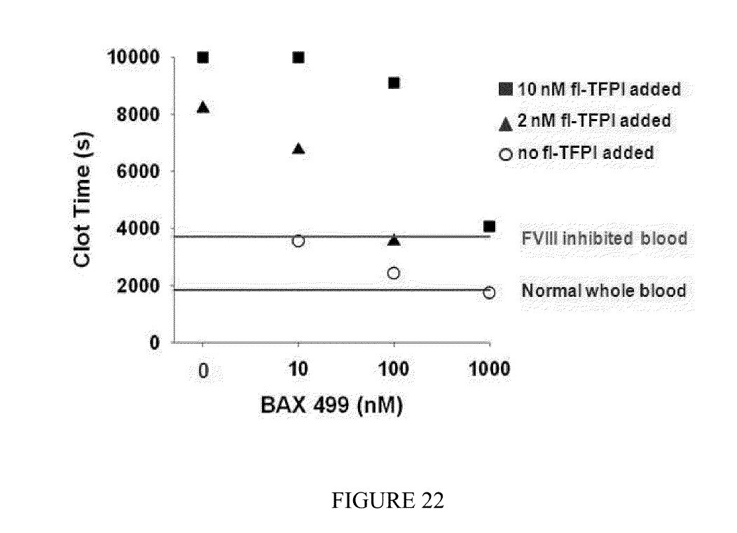

Exemplary results are illustrated in FIGS. 21 and 22. Addition of fl-TFPI to FVIII inhibited whole blood in the absence of BAX 499 substantially inhibited coagulation as indicated by a marked increase in clot time (FIGS. 21 and 22). BAX 499 improved coagulation by reducing the clot time to normal levels in a concentration dependent manner (FIG. 22; open circles). However, BAX 499 hardly reached the clot times of FVIII inhibited blood to which TFPI was added (e.g. 10 nM). This indicates that BAX 499 does not neutralize TFPI at increased TFPI concentrations.

FIG. 21 illustrates the procoagulant effect of BAX 499 (0 nM to 1000 nM) in FVIII inhibited whole blood in absence (0 nM human fl-TFPI) and presence of increasing amounts of added fl-TFPI (2, 10 nM). ROTEM tracings of FVIII inhibited and normal whole blood are shown as reference.

FIG. 22 illustrates the procoagulant effect of increasing concentrations of BAX 499 (10, 100, 1000 nM) in FVIII inhibited whole blood in absence (no fl-TFPI added, open circles) and presence of increasing amounts of added fl-TFPI (2 nM, closed triangles; 10 nM, closed squares). Clot times of FVIII inhibited and normal whole blood are shown as reference.

### Example 10

### A Biochemical Analysis of BAX 499

### Materials

Human coagulation factors II, VII, IX and X were isolated from fresh frozen plasma using the methods of Bajaj et al. [Prep Biochem. 1981; 11: 397-412] and Jenny et al. [Prep Biochem. 1986; 16: 227-45]. The proteins were purged of trace contaminants and traces of active enzymes as described [van't Veer C et al. J Biol. Chem. 1997; 272: 4367-7]. FX was activated to FXa in house [Jesty J. et al. Methods Enzymol. 1976; 45: 95-10]. Human FV [Katzmann J A et al. Proc Natl Acad Sci USA. 1981; 78: 162-6] and antithrombin [Griffith M J et al. J Biol. Chem. 1985; 260: 2218-25] were isolated from freshly frozen citrated plasma. 1,2-Dioleolyl-sn-Glycero-3-Phospho-L-Serine (PS) and 1,2-Dioleoyl-sn-Glycero-3-Phosphocholine (PC) were purchased from Avanti Polar Lipids (Alabaster, Ala.). TF was relipidated in PCPS (75% PC:25% PS) vesicles (1:2000 protein/lipid) by a previously described protocol [Barenholz Y et al., Biochemistr. 1977; 16: 2806-10; Lawson J H et al. Methods Enzymol. 1993; 222: 177-95]. HEPES, sodium chloride, calcium chloride, polyethylene glycol (PEG) and EDTA were purchased from Fisher Scientific (Pittsburgh, Pa.). Corn trypsin inhibitor (CTI) was isolated as previously described [Hojima et al. Thromb Res. 1980; 20: 149-62]. Spectrozyme Xa and Spectrozyme TH were purchased from American Diagnostica (Stamford, Conn.). The monoclonal inhibitory antibody α-FIX-91 was produced and characterized as previously described [Butenas S. et al. Blood. 2002; 99: 923-30]. Single-chain t-PA was separated from a mixture of single-chain and two-chain t-PA as previously described [Butenas S et al. Biochemistry. 1997; 36: 2123-31]. D-Phe-Pro-Arg-CH₂Cl (FPRck) and the fluorogenic substrate 6-(D-Phe-Pro-Arg)-amino-1-naphthalenebutylsulfonamide (FPRnbs) was produced as previously described [Butenas S et al., Biochemistry. 1992; 31: 5399-411. Butenas S. et al., J Biol. Chem. 1997; 272: 21527-33]. The fluorogenic substrate Z-GGR-AMC was purchased from Bachem (Torrance, Calif.).

### FXa Inhibition by TFPI

The active concentration of TFPI was determined by a titration using FXa [Girard T J et al., Methods Enzymol. 1993; 222: 195-209]. It was found to be 60% of the total protein mass present. All TFPI concentrations stated represent the active concentration.

Characterization of the reaction between TFPI and FXa in the presence or absence of BAX499 was performed as described by Baugh et al. [Baugh R J et al., J Biol. Chem. 1998; 273: 4378-86]. TFPI (3 nM) was added to a mixture of human FXa (1 nM) and BAX499 (0-250 nM) in HEPES buffered saline (HBS)/0.1% PEG/2 mM CaCl₂ at 37° C. At predetermined time intervals, aliquots were removed and added to 200 μM Spectrozyme Xa. The reaction was diluted only 10% by the chromogenic substrate. The absorbance at 405 nm was read immediately in a THERMOmax microplate reader (Molecular Devices Corp., Menlo Park, Calif.). Initial velocity measurements were used to estimate the free or uninhibited concentration of FXa remaining at each sampled time point by reference to a calibration curve.

To estimate a Kd value for the interaction between TFPI and BAX499, it was assumed that the final distribution of species when TFPI, FXa and aptamer are mixed is defined by two competing reversible interactions involving 1 to 1 complexes between FXa and TFPI (equation 1) and TFPI and aptamer (equation 2).

The relevant equations are:

1: Kd=[TFPI]free*[FXa]free/[FXa−TFPI]

2: FXatotal=FXafree+FXa−TFPI

3: TFPItotal=TFPIfree+FXa−TFPI+BAX499−TFPI

4: BAX499total=BAX499free+BAX499−TFPI

5: Kd=[TFPI]free*[BAX499]free/[BAX499−TFPI]

The time course of FXa inhibition for each aptamer concentration was analyzed using double exponential fits to estimate the free FXa concentration present at equilibrium. Using the measured [fXa]free and a Kd value of 0.4 nM [Baugh R J et al., J Biol. Chem. 1998; 273: 4378-86], equations 1 and 2 were used to calculate [TFPI]free, then equation 3 was used to calculate [BAX499-TFPI], and equation 4 was used to calculate [BAX499]free. The apparent Kd for the TFPI aptamer interaction at each concentration of aptamer was then calculated using equation 5.

### Inhibition of TF/FVIIa by TFPI

2 nM TF, 4 nM FVIIa and variable BAX499 (0-15 nM) (all at 2× final concentration) was incubated at 37° C. for 10 minutes. This mixture was diluted 2-fold with 100 μM FBRnbs±24 nM TFPI (both at 2× final concentration). The reaction was then read for 5 minutes in a Synergy4 plate reader (Bio-Tek, Winooski, Vt.) with excitation of 350 nm and emission of 470 nm. Activity present in the sample with TFPI was subtracted from the control without TFPI for each BAX499 concentration.

### FX Activation

20 pM relipidated TF, 1 nM FVIIa, 20 μM PCPS, and aptamer (0-1000 nM) in HBS/0.1% PEG/2 mM CaCl₂ was incubated for 10 min at 37° C. 250 nM FX and 3 nM TFPI were then added to the reaction mixture. Every minute, an aliquot was removed and quenched into 15 mM EDTA and 200 μM Spectrozyme Xa. Aliquots were diluted only 15% by the quenching solution. The absorbance at 405 nm was read immediately in a THERMOmax microplate reader.

### Synthetic Coagulation Proteome

The procedure used is a modification of Lawson et al. [J Biol. Chem. 1994; 269: 23357-66] and van't Veer et al. [J Biol. Chem. 1997; 272: 4367-77]. Relipidated TF at 5 pM and PCPS at 50 μM (final concentrations) were added to a mixture of procoagulant factors II, V, VII, VIIa, VIII, IX and X, as well as inhibitors TFPI and AT (all at mean physiologic concentrations) in HBS/0.1% PEG/2 mM CaCl₂ at 37° C. When desired, BAX499 (0-10 nM) was added and FVIII or FIX was omitted. Every minute, an aliquot was removed and added to 20 mM EDTA and 200 μM Spectrozyme TH. The absorbance at 405 nm was read immediately in a THERMOmax microplate reader. The thrombin concentration was determined by a calibration curve constructed using dilutions of α-thrombin.

### Thrombin Generation Assay (TGA)

TGAs were performed as previously described [Mann K G et al., J Thromb Haemost. 2007; 5: 2055-61]. All assays utilized contact pathway inhibited (CTI, 0.1 mg/mL) citrated pooled normal plasma either in the absence or presence of 0.1 mg/mL α-FIX-91 to simulate hemophilia B. 80 μL of plasma (premixed with the desired concentration of BAX499) was mixed with 20 μL HBS containing 90 mM CaCl₂ and 2.5 mM fluorogenic substrate Z-GGR-AMC and incubated for 3 minutes at 37° C. The reaction was initiated by the addition of 20 μL HBS containing 30 pM TF and 120 μM PCPS. Thrombin generation was monitored in the Synergy4 plate reader.

### Whole Blood Clotting Assay

The procedure used was a modification of Rand et al. [Blood. 1996; 88: 3432-45]. Blood was collected by venipuncture and immediately placed into a beaker containing CTI (100 μg/mL)±α-IX (100 μg/mL)±BAX499 (100 nM). This blood was then aliquoted (1 mL) into rocking tubes at 37° C. containing TF (5 pM or 1 pM) relipidated in PCPS. Tubes were quenched at defined time intervals to 24 minutes with a 1 mL volume of a mix of inhibitors (50 mM EDTA, 20 mM benzamidine, 100 μM FPR-ck). All samples were centrifuged (4° C., 1200 g, 30 min), and the soluble material was frozen at −80° C. until further analysis (in-house) for α-thrombin-antithrombin (α-TAT) complex [Foley J H et al. Thromb Res. 2012].

### Thromboelastography (TEG)

Blood was collected by venipuncture and added to 100 μg/mL CTI±100 μg/mL α-FIX-91. 345 μL were added to a TEG cup containing relipidated TF (0.5 pM final), single chain t-PA (1 nM final)±BAX499 (100 nM final). Analysis was carried out on each sample using a Thromboelastograph Haemostasis Analyzer Model 5000 (Haemoscope, Niles, Ill.) at 37° C. TEG parameters were extracted using TEG V4 software (Haemoscope, Niles, Ill., USA).

### Results

### Aptamer Blockade of TFPI Inhibition of FXa

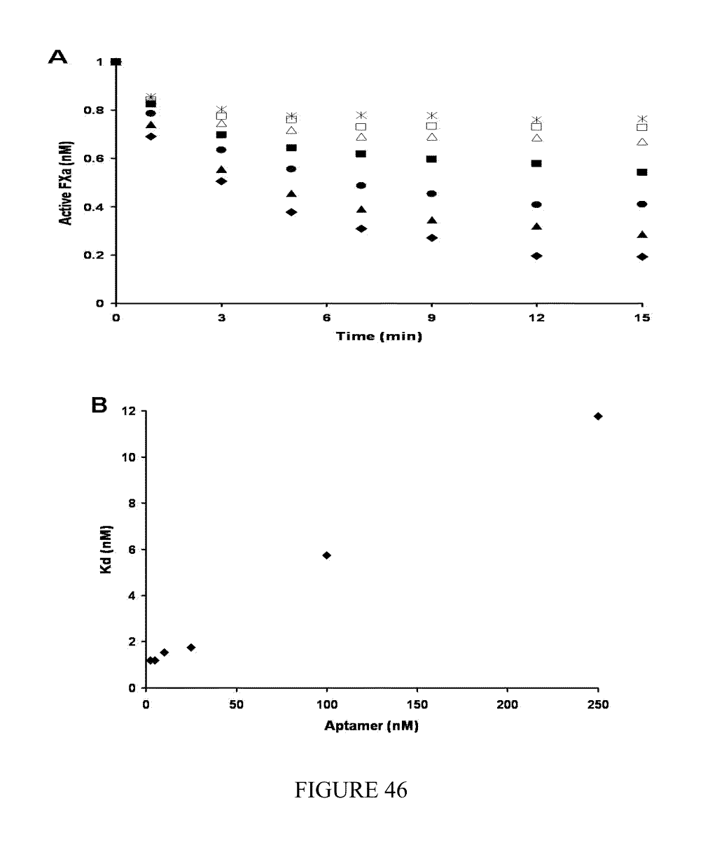

At a concentration of up to 500 nM, BAX499 had no effect on FXa activity (data not shown) in the absence of TFPI. In the absence of BAX499, TFPI decreased the concentration of active FXa from 1 nM at zero time to 0.19 nM after 15 minutes (FIG. 46A). Analysis of this binding isotherm indicated that the Kd for TFPI and FXa was ˜0.4 nM. With increasing BAX499, the effect of TFPI was lessened in a concentration dependent manner. At 250 nM aptamer, 0.76 nM FXa remained after 15 minutes.

Using double exponential fits to analyze each binding isotherm, a dissociation constant was calculated for each concentration of aptamer (FIG. 46B). Between 2.5 and 25 nM BAX499, the calculated Kd ranged between 1.2 and 1.8 nM. However, the calculated Kd increased at higher aptamer concentrations. At 100 nM aptamer, the calculated Kd was 5.8 nM. At 250 nM aptamer, the calculated Kd increased to 11.8 nM.

### Aptamer Blockade of TFPI Inhibition of TF/FVIIa

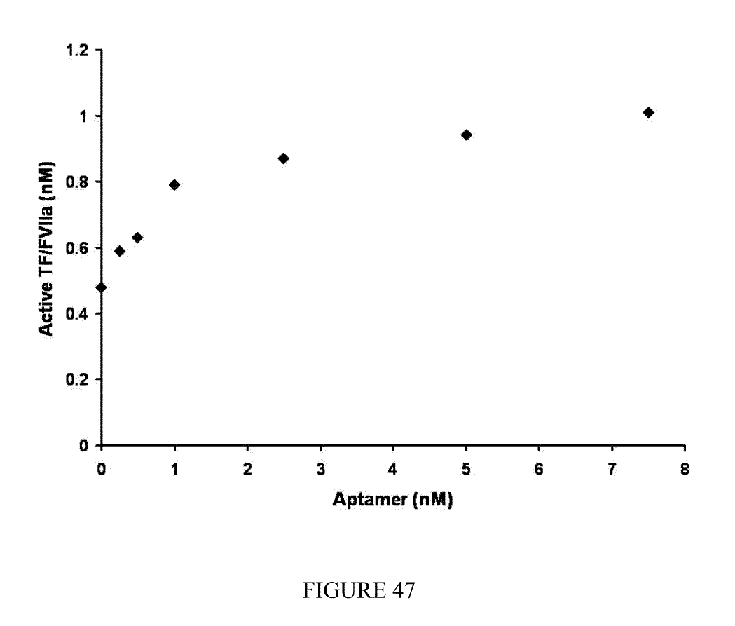

Characterization of the effect of BAX499 on FXa independent TFPI inhibition of TF/FVIIa was performed using a lipid independent fluorogenic assay that directly measures TF/VIIa cleavage rate of a small substrate [Butenas S et al., Biochemistry. 1992; 31: 5399-411]. In the absence of BAX499, 12 nM TFPI inhibited 48% of the TF/FVIIa activity (FIG. 47). BAX499 decreased apparent TFPI efficacy in a concentration dependent manner. At 7.5 nM BAX499, the TF/FVIIa activity was equivalent to that seen in the absence of TFPI. The calculated apparent Kd of BAX499 for TFPI in this assay was ˜1.2 nM.

### Aptamer Blockade of TFPI Inhibition of Extrinsic FXase Activity

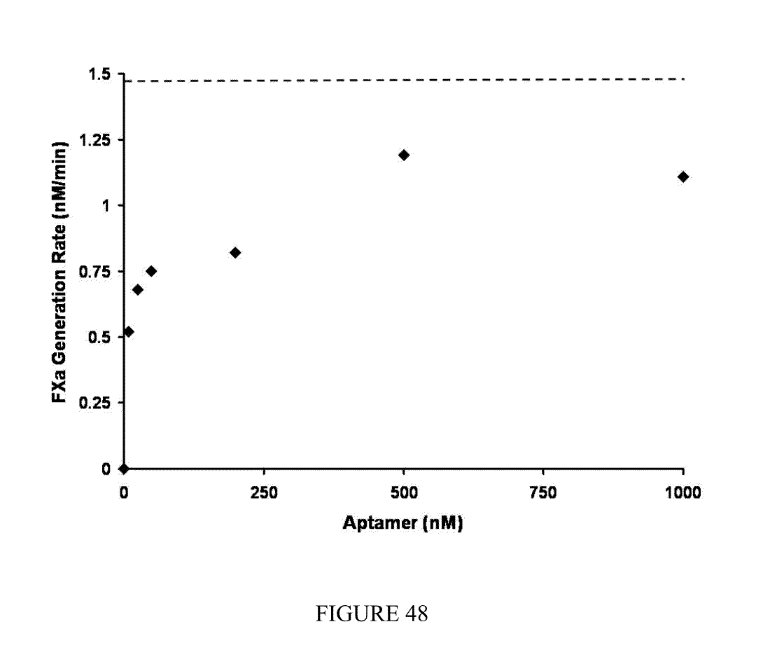

At a concentration of 500 nM, BAX499 had no effect on FXa activation by the TF/VIIa complex in the absence of TFPI (FIG. 48, dotted line). In the absence of BAX499, the addition of 3 nM TFPI caused the FXa generation rate to be below the detection limit of our assay (<0.05 nM/min). With increasing BAX499 concentration, FXa generation increased. At 50 nM aptamer, about 50% of the FXase activity was recovered. However, saturating levels of BAX499 (˜500 nM) resulted in a FXa generation rate 75-80% of that observed in the absence of TFPI.

### Aptamer Modulation of TFPI Activity in TF-Initiated Thrombin Generation

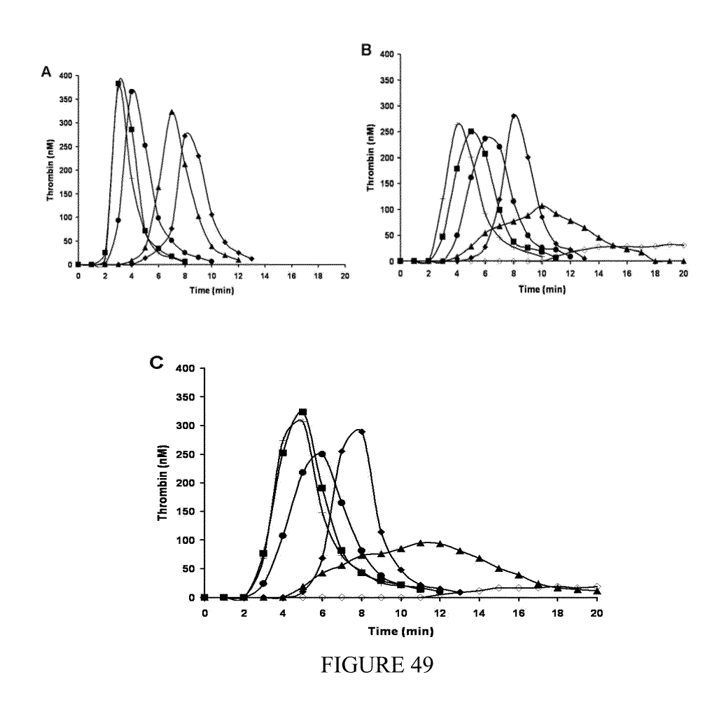

Synthetic coagulation proteome. In the synthetic coagulation proteome model, the onset of thrombin generation in the control experiment (100% FVIII, 100% FIX) with no BAX499 occurred between 4 and 5 minutes (FIG. 49A). Addition of 1 nM BAX499 decreased this time to 3-4 minutes. 5 nM BAX499 further decreased the onset to 2-3 minutes. The addition of 10 nM BAX499 resulted in a thrombin generation curve that was similar to that observed when TFPI was omitted from the system. The maximum level of thrombin generated similarly increased in a concentration dependent manner, from 270 nM with no aptamer to 380 nM in the presence of 10 nM aptamer.

BAX499 was then tested in hemophilia proteome models. In the absence of FVIII, the onset of thrombin generation occurred at 10 minutes and reached a maximum level of 30 nM (FIG. 49B) compared to 4-5 minutes and 270 nM, respectively, in the control. The addition of 1 nM BAX499 decreased the onset time to 4 minutes and the maximum level of thrombin was increased to 110 nM. An increase to 5 nM BAX499 resulted in a faster onset of thrombin generation curve than in the control (100% FVIII), reaching a maximum level of 240 nM. A further increase to 10 nM BAX499 resulted in thrombin generation dynamics similar to that seen in the absence of TFPI. Comparable results were seen when BAX499 was evaluated in the proteome model of hemophilia B (absence of FIX; FIG. 49C).

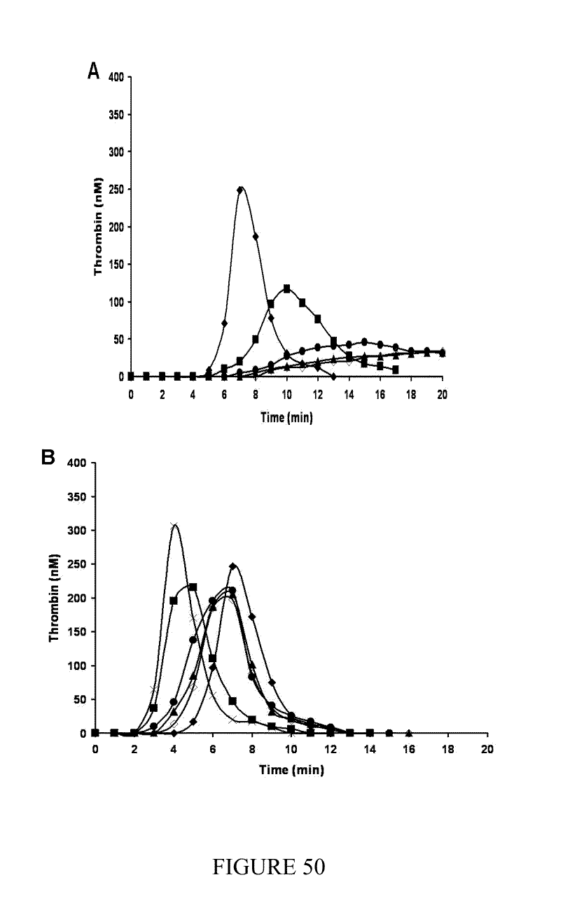

BAX499 at FVIII concentrations seen in moderate to mild hemophilia were then investigated. A titration without BAX499 is shown on FIG. 50A. FVIII concentrations of less than 5% were characterized by slow thrombin generation (onset of 7-9 minutes) that had a maximum level of less than 50 nM. At 40% FVIII, the onset of thrombin generation occurred at 6 minutes, and the maximum level reached 120 nM. The control experiment with 100% FVIII reached a maximum level of approximately 250 nM and had an onset time of 5 minutes.

In the experiments of FIG. 50B, FVIII was titrated with 2.5 nM aptamer present in the proteome. When FVIII concentrations were 0%, 2% or 5% of the mean physiologic level, the presence of 2.5 nM BAX499 resulted in nearly identical thrombin generation curves. These profiles were characterized by an earlier onset of thrombin generation and maximum thrombin levels of 80-90% of that observed with the 100% FVIII control. At 40% FVIII with 2.5 nM BAX499, the onset of thrombin generation occurred at approximately 2 minutes and resulted in nearly an identical maximum level to the 100% FVIII control.

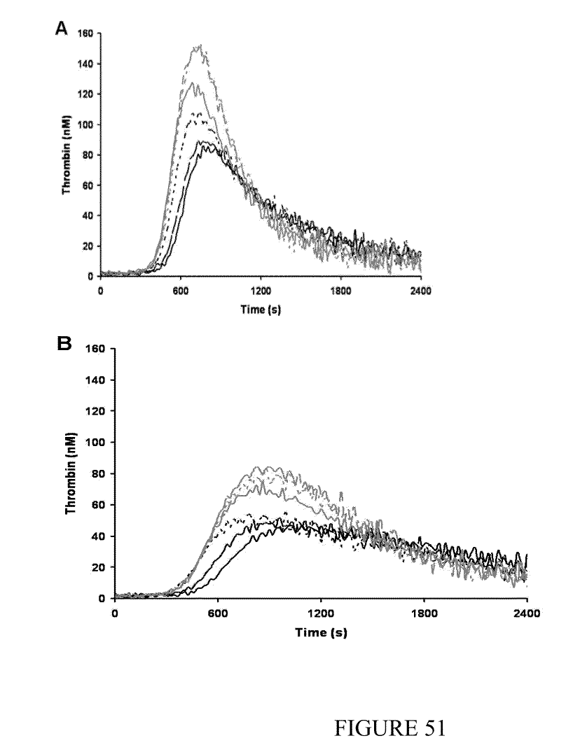

Thrombin generation assay (TGA). FIG. 51 presents an analysis of aptamer efficacy in normal and induced hemophilia B plasma. In the TGA, the control (no α-FIX-91, FIG. 51A) experiment with no BAX499 reached a maximum of 85 nM, a concentration that was unchanged with the addition of 1 nM BAX499. An increase to 10 nM aptamer raised the maximum thrombin level to 105 nM. 100 nM BAX499 caused the maximum level to increase to 125 nM. A maximum of 150 nM thrombin was reached with 500 nM BAX499. An increase in aptamer concentration to 1 μM did not increase the maximum thrombin level beyond 150 nM thrombin.

In induced hemophilia B plasma (FIG. 51B), levels of BAX499 at or below 10 nM did not increase peak thrombin from 50 nM, the level that was observed in the absence of the aptamer (although the rate did increase). A maximum thrombin level of 75 nM was reached at 100 nM aptamer. At aptamer levels of 500 nM, the maximum level was 85 nM, nearly indistinguishable from the control (without α-FIX-91 or BAX499). An increase to 1 μM BAX499 did not amplify thrombin generation further. In the plasma milieu, normalization of thrombin generation in hemophilia B required over 100 times more BAX499 compared to the buffer system.

### Aptamer Modulation of TFPI Activity in TF-Initiated Coagulation in Contact Pathway Inhibited Whole Blood

Thrombin Generation.

Initial experiments were conducted using a 5 pM TF stimulus in contact pathway inhibited whole blood. In contrast to proteome and plasma model systems, aptamer concentrations up to 1 μM failed to improve thrombin generation (α-thrombin-antithrombin complex, α-TAT) in the induced hemophilia B model (data not shown). A subsequent TF titration in induced hemophilia B blood indicated that at TF concentrations less than or equal to 1 pM, the aptamer did affect thrombin dynamics, shifting clot times towards the control value (no α-FIX-91, data not shown).

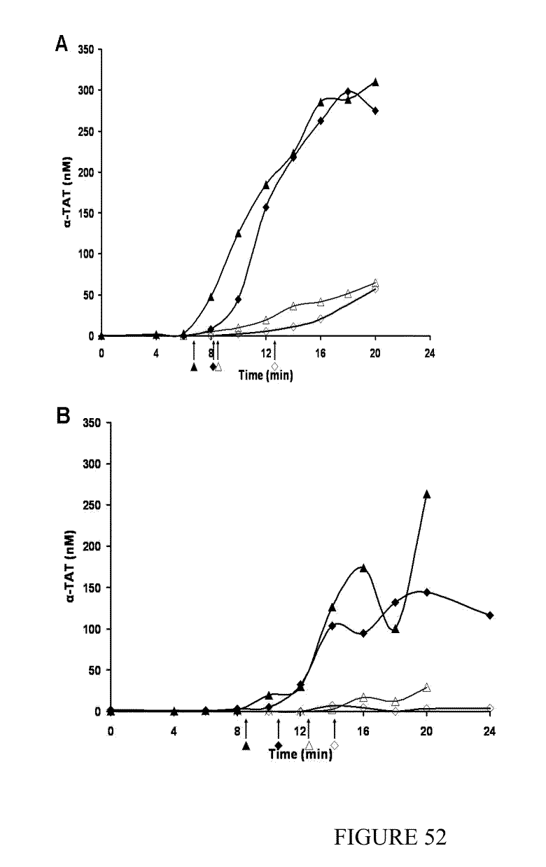

FIG. 52 presents α-TAT formation in blood from two individuals in which whole blood clotting was initiated by 1 pM TF in the presence and absence of 100 nM BAX499. The effect of this aptamer was tested in both normal and induced hemophilia B models. The addition of the aptamer to the control experiment decreased the clotting time from 8.3 to 6.7 minutes in subject 1 (panel A) and from 10.6 to 8.5 minutes in subject 2 (panel B). In neither subject did the effect of the aptamer increase the rate or extent of α-TAT formation.

In both individual's blood, aptamer addition to their induced hemophilia B blood reduced the clotting time. In subject 1, the clotting time was reduced from 12.8 to 8.1 minutes, which was similar to the control experiment (8.3 minutes). In subject 2, the aptamer reduced the clotting time from 14.0 to 12.1 minutes.

However, similar to results seen in control blood, the aptamer did not markedly improve α-TAT generation in the hemophilia setting. In subject 1, the 20 minute level of α-TAT for the control experiment was 310 nM. This value decreased to 60 nM in the presence of α-FIX-91, a level that was unchanged in the presence of 100 nM BAX499. The results for subject 2 similarly showed minimal improvement in α-TAT generation in the induced hemophilia B setting.

### Thromboelastography

When analyzed by thromboelastography, hemophilia blood can display prolonged R values (clot time) and reduced angle parameter values (rate of clot growth) relative to normal blood [Othman M. et al, Haemophilia. 2009; 15: 1126-34; Young G. et al., Haemophilia. 2006; 12: 598-604]. An analysis in contact pathway inhibited, induced hemophilia B fresh phlebotomy blood was performed using a defined TF (5 pM) stimulus. The presence of 100 nM BAX499 restored the TEG profile of induced hemophilia B to that seen in a control (data not shown).

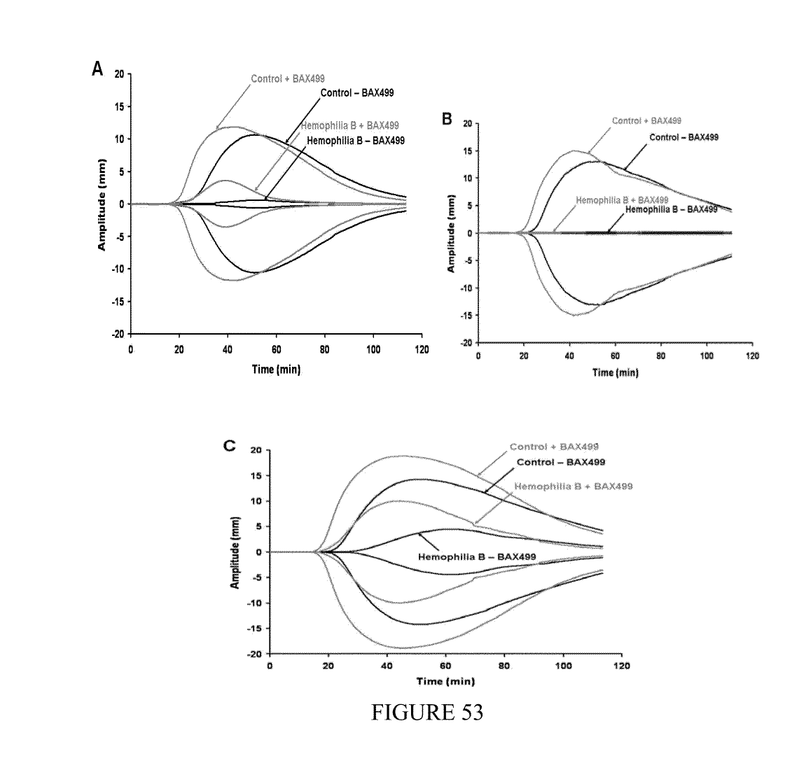

The inclusion of t-PA in TEG assays has been used to quantify differences in clot stability in hemophilia in response to various hemostatic agents [Hvas A M et al., J Thromb Haemost 2007; 5: 2408-14; Foley J H et al., Blood. 2012]. FIG. 53 presents the results for three individuals of a viscoelastic analysis of aptamer effects on clot stability. Coagulation in contact pathway inhibited blood from 3 healthy subjects was initiated with 0.5 pM TF in the presence of 1 nM t-PA in normal or induced hemophilia B systems. The TF concentration was chosen to maximize the differences seen between control and induced hemophilia blood. Table 8 summarizes the resulting TEG parameters: each subject's contact pathway inhibited blood supplemented with 1 nM t-PA was analyzed in the presence or absence of 100 nM BAX 499 and in the presence or absence of inhibitory antibody α-FIX-91. R is clot time, MA is maximum amplitude, and UD is undefined.

TABLE 8
Thromboelastographic analysis of BAX 499

| | Control | | | | Induced Hemophilia B | | | |
|---|---|---|---|---|---|---|---|---|
| | −100 nM | | +100 nM | | −100 nM | | +100 nM | |
| | BAX499 | | BAX499 | | BAX499 | | BAX499 | |
| Subject | R | MA | R | MA | R | MA | R | MA |
| 3 | 26 ± 4 | 21 ± 5 | 20 ± 0 | 23 ± 6 | UD | UD | 26 ± 1 | 7 ± 1 |
| 4 | 25 ± 1 | 26 ± 2 | 20 ± 0 | 30 ± 3 | UD | UD | UD | UD |
| 5 | 23 ± 1 | 28 ± 3 | 17 ± 0 | 37 ± 2 | 35 ± 1 | 8 ± 2 | 21 ± 1 | 20 ± 5 |

In all three subjects, the addition of 100 nM BAX499 shortened the R time in control (no α-FIX-91+1 nM t-PA) blood by an average of 6 minutes, while maximum amplitude (MA) values were relatively unchanged (FIG. 53, Table 8). R times in induced hemophilia B blood (+1 nM t-PA) were increased relative to the control, with subjects 3 (panel A) and 4 (panel B) having an undefined R time (>120 minutes). All subjects' MA values were strongly decreased (or undefined) in hemophilia blood relative to their controls.

With the addition of 100 nM BAX499 to induced hemophilia B blood, two of the three subjects displayed a normalized R time. Subject 4's undefined R time remained undefined despite the presence 100 nM BAX499. The effect of BAX499 on the MA parameter in induced hemophilia B blood varied among the subjects, with only subject 5 (panel C) showing a value approaching that of the untreated control.

In two subjects (subjects 3 and 5), the addition of 100 nM BAX499 generated a clot in induced hemophilia blood faster than without the aptamer. However, the clots did not appear to have lasting strength. In both cases, the clots were solubilized faster in induced hemophilia blood with BAX499 than in the hemophilia control.

This example analyzes BAX499 mechanism, specificity and potency in experimental systems with purified components, in contact pathway-inhibited citrate plasma and in contact pathway-inhibited whole blood. In entirely synthetic systems of purified proteins, the aptamer BAX499 specifically and effectively neutralized TFPI function, including direct TFPI inhibition of FXa and TF/FVIIa, and its FXa dependent inhibition of TF/FVIIa. One finding was the increase in calculated apparent Kd for BAX499 and FXa with increasing aptamer concentration.

In hemophilia models (permitting direct measurements) directly measuring thrombin generation after TF initiation, BAX499 proved effective in restoring the defect in thrombin generation. In the synthetic coagulation proteome models of hemophilia A and B, just 1-5 nM BAX499 was sufficient to mitigate the delayed onset and suppressed thrombin generation associated with hemophilias A and B. Compared to the buffer-based coagulation proteome, contact pathway inhibited plasma thrombin generation assays required a higher concentration of BAX499 (˜100 nM) to restore the induced hemophilia B system to what was seen in the control.

A TEG-based analysis of BAX499 efficacy in contact pathway inhibited induced hemophilia B blood subjected to a well-defined 5 pM TF stimulus (data not shown). However, in two additional models of TF dependent induced hemophila B blood coagulation, BAX499 restored the clot time parameter while failing to normalize the overall response to TF (FIGS. 52 and 53).

In a modified TEG assay with t-PA, significant individual variation was noticed. This assay was designed to provide a more comprehensive assessment of a hemophilia individual's capacity to form a stable clot by simultaneously challenging each individual's procoagulant and fibrinolytic systems. Assayed in this way, the three subjects showed three different levels of response to the α-FIX-91 antibody and to BAX499. However, in only one case did the aptamer restore hemophilia profiles close to the state of its corresponding control. Effective clot formation in this in situ model presumably reflects the overall balance between procoagulant and fibrinolytic processes. These data suggest that variation between individual blood donors can contribute to outcomes with BAX499, with its effects on clot time restoration more universal then its capacity to normalize thrombin generation and clot stability.

### Example 11

### BAX 499 Restores Clot Formation to Normal Levels and Reduces Fibrinolysis in Lysis-Induced FVIII-Inhibited Whole Blood

The impact of BAX 499 on hemostasis and fibrinolysis was determined with low tissue factor-triggered rotational thromboelastometry (ROTEM®) in FVIII-inhibited (AFVIII), tissue plasminogen activator (tPA)-induced human whole blood.

### Methods

Continuous visco-elastic assessment of whole blood clotting with a ROTEM® coagulation analyzer (Pentapharm) was performed. Principle is shown in FIG. 67A. FVIII activity was antibody-inhibited with heat-inactivated goat plasma in freshly collected citrated normal whole blood (50 Bethesda units/mL) to generate a model of hemophilia blood. Each sample was measured by pre-warming 20 μL of 0.2 M CaCl₂ and 20 μL of diluted tissue factor (TF) PRP-reagent (Thrombinoscope BV) in a ROTEM® cuvette at 37° C. to give final concentrations of 11.76 mM and 44 fM, respectively. 4 μL of BAX 499 (5-1000 nM) or buffer substitute was added to 300 μl of pre-warmed blood 10 s prior to measurement.

To study fibrinolysis, tPA (Actilyse, Boehringer Ingelheim) was added at a final blood concentration of 90 ng/ml and a final TF concentration of 0.2 pM to the reagent mix. The ROTEM® recording was started immediately and proceeded for at least 100 min. The ROTEM® parameters such as clotting time (CT) and maximum clot firmness (MCF) were recorded in accordance with the manufacturer's instructions. The ROTEM® analyses were performed in the presence of 1 μM BAX 499 and the area under curve (AUC) of each ROTEM® tracing was calculated by using the raw data export tool, Excel and Sigma Plot 12 software. Additional data analysis is described in FIG. 67B.

### Results

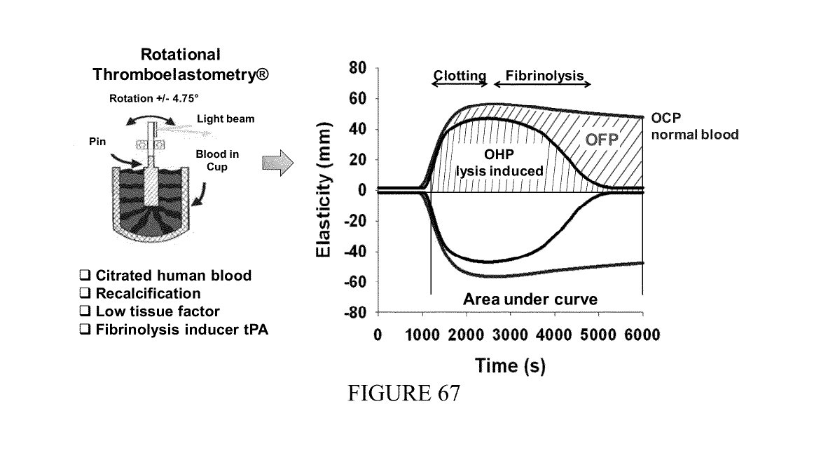

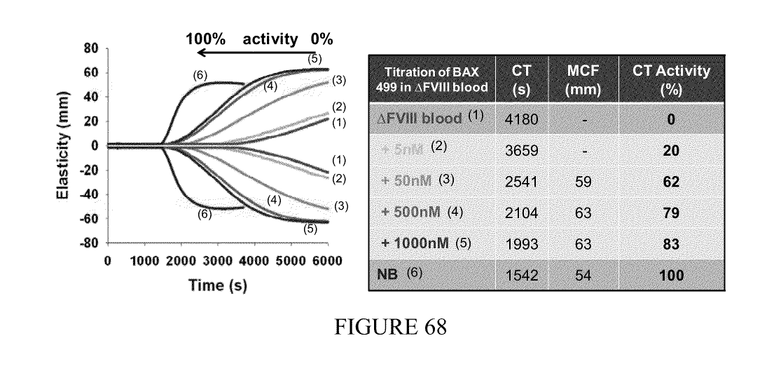

A very low TF concentration was used to trigger whole blood coagulation to make conditions sensitive to TFPI and to FVIII. BAX 499 shortened CT and corrected MCF to normal in AFVIII whole blood. The effect was concentration dependent and saturated at 1 μM BAX499 (FIG. 68).

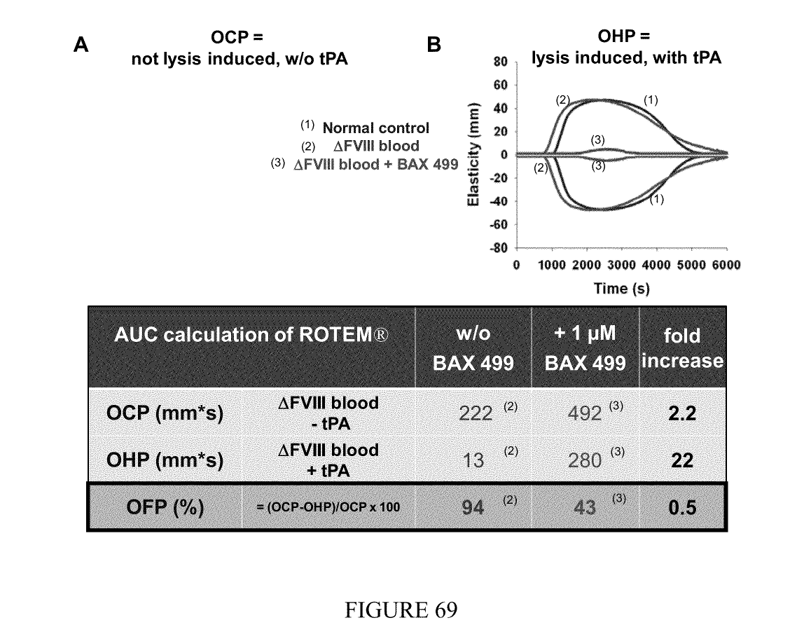

Fibrinolysis induced AFVIII whole blood was used to measure the effect of BAX 499 on hemostasis. The OFP of lysis induced AFVIII whole blood was 94%, while the addition of BAX 499 decreased the OFP more than half to 43%, as an indication for improved coagulation and reduced fibrinolysis. In the absence of exogenous tPA, BAX 499 caused a 2-fold increase in the OCP of AFVIII whole blood, restoring coagulation to more than normal levels. The hemostatic effect of BAX 499 was further increased by 20-fold in lysis-induced AFVIII whole blood, reflecting its OHP (FIG. 69).

### Example 12

### The Effect of BAX 499 on Blood Coagulation in the Presence of Different Concentrations of Factor VIII

### Patient Selection and Blood Collection

Blood was collected from hemophilia A patients with their informed consent under a protocol approved by the Center for Theoretical Problems of Physicochemical Pharmacology and National Research Center for Hematology Ethical Committees. A total of nine patients with severe hemophilia A having baseline FVIII concentrations less than 1% participated in the pharmacokinetics study, and three patients donated blood for control experiments. All patients were under therapy with different FVIII concentrates (Haemoctin, Kogenate or Octanate), but they did not receive FVIII concentrates for three days before the experiment. Blood samples were drawn before Factor VIII administration (0 h) and at 1 and 24 h timepoints into 3.8% sodium citrate (pH 5.5) at a 9/1 blood/anticoagulant ratio, in the presence of CTI (0.1 mg/ml final concentration).

### Plasma Preparation

Within 10 minutes of sample collection, blood was processed by centrifugation at 2,500 g for 15 min to obtain platelet-poor plasma, and then additionally centrifuged at 11,000 g for 5 min to obtain platelet-free plasma and frozen at −80° C. for further spatial coagulation assays.

### Experimental Design of the Spatial Model

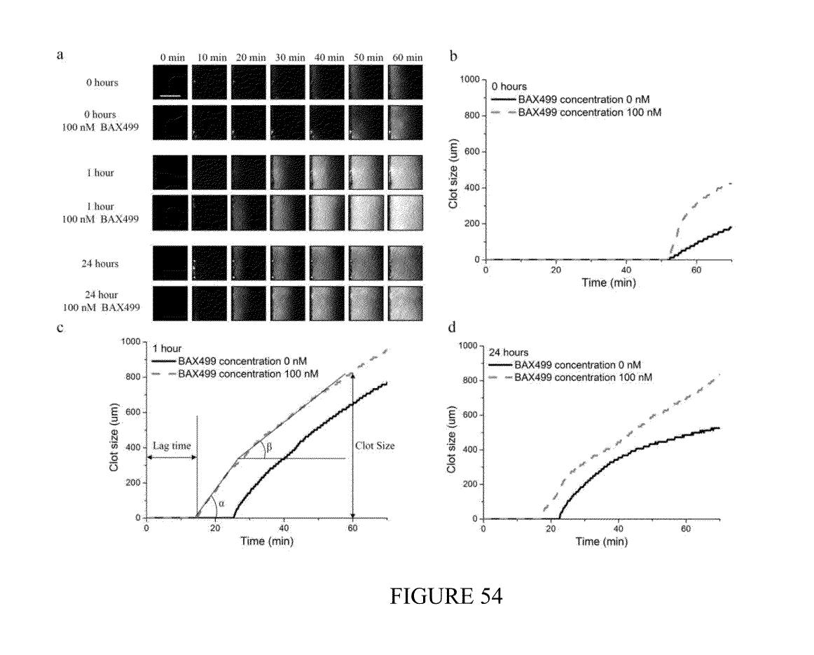

The key property of the spatial experimental model is that plasma clotting is activated by a surface covered with immobilized TF and then propagates into the bulk of plasma as shown in FIG. 54a. Clotting took place in a thin flat chamber at 37° C. and was registered by light scattering from fibrin gel using dark field technique (Ovanesov et al, Biochim. Biophys. Acta, 1572, 45-57 (2002); Ovanesov et al, J. Thromb. Haemost., 3, 321-331 (2005)). Images were captured every 15 s. The acquired series of images was then processed by computer and parameters of spatial dynamics of blood clotting are calculated. Thromboplastin was immobilized on a polystyrene surface by chemical sorption method essentially as described (Fadeeva et al, Biochemistry (Mosc.), 75, 734-743 (2010)).

### Spatial Clot Formation Assay

Before the experiment, plasma was thawed for 10 min under flowing water, and plasma pH was stabilized at 7.2-7.4 by lactic acid treatment as described (Sinauridze et al, Biochim. Biophys. Acta, 1425, 607-616 (1998)). BAX499 aliquots were thawed at room temperature for 30 min before the first experiment of the day. BAX499 was dissolved in PBS to achieve the necessary final concentration. At 15 min before each experiment, 300 μl of plasma was supplemented with 4.5 μl of BAX499 solution. In control experiments with a final concentration of 0 nM of BAX499, plasma was supplemented with the same volume of vehicle PBS. Activator was placed into buffer (20 nM HEPES, 150 mM of NaCl, pH=7.2-7.4) to reduce bubble formation near the activator during the experiment. The solution of 1 M CaCl₂, buffer with activator and prepared plasma were incubated separately at 37° C. for 15 minutes. The experimental chamber was placed into the thermostat of the experimental device at 37° C. Plasma was subsequently decalcified by addition of 6 μl 1M CaCl₂, quickly mixed and 300 μl of plasma was placed into the experimental chamber. The activator was taken out of the buffer, buffer excess was removed by blotter, and the activator was placed into the experimental chamber to start clotting. Spatial fibrin clot growth was recorded as described above.

### Image Processing and Data Analysis

For each experiment, parameters of clot growth were determined on the basis of image series. First, background image was subtracted from each image of the series, and the resulting images were analyzed. A perpendicular to the activator was drawn and clot profiles, plots of mean light scattering (based on pixel intensity) versus distance from the activator, were calculated. For each profile, the clot size was determined as a coordinate where the light-scattering intensity was 50% of the maximal one, which corresponded to half-maximal fibrinogen conversion into fibrin (Ovanesov et al, J. Thromb. Haemost., 3, 321-331 (2005)). As shown in FIG. 54b, based on clot size versus time plots, the following parameters were calculated: lag time (delay between contact of plasma with activator and beginning of clot formation), initial velocity of clot growth (mean slope of the clot size versus time curve over the first 10 min after the lag time), spatial velocity of clot growth (mean slope over the next 30 min), clot size after 60 min of the experiment. For each experiment, four perpendiculars to the activator surface were drawn at different areas of activator. Profiles of clot growing were analyzed and four values of values of each clotting parameter were calculated and then averaged to obtain means.

### Mathematical Modeling

Computer simulations of blood clotting were carried out using a detailed mechanism-driven mathematical model (Panteleev et al, Biophys. J., 90, 1489-1500 (2006)) of clotting in a reaction-diffusion system with minor modifications.

### Results

### Experimental Design

Typical spatial fibrin clot formation experiments for hemophilia A plasma with different factor VIII and BAX499 levels are shown in FIG. 54. The image series in FIG. 54a show fibrin clots observed with and without 100 nM of BAX499 in plasmas collected at different time points after factor VIII administration. FIGS. 54b-d show clot size as a function of time for the same experiments; in particular, FIG. 54c demonstrates clot formation parameters used: lag time, initial (tg α) and stationary (tg β) spatial clot growth velocity, and clot size at 60 minute after the beginning of the experiment. It can be seen that spatial clot formation was detectably improved by BAX499 in hemophilia A plasma. Addition of BAX499 at 100 nM accelerated clotting onset, so that lag time became shorter and final clot size became larger.

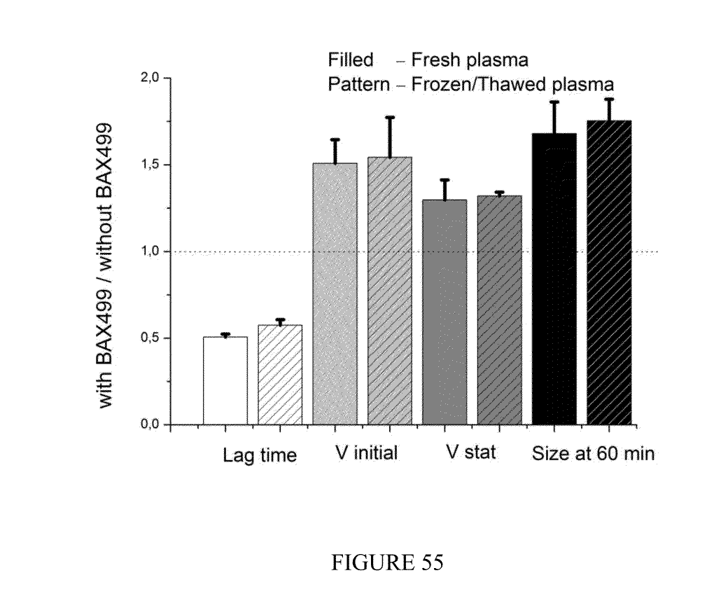

To test the possibility that freezing/thawing plasma can influence the BAX499 effects, experiments were performed with and without BAX499 using fresh and frozen/thawed plasma from three hemophilia A patients. The ratios for the main analyzed parameters (lag time, velocities, clot size) with 300 nM of BAX499 to baseline (without BAX499) were determined. The data in FIG. 55 suggested that, based on Student t-test with p=0.95, there was no significant difference in the BAX499 effect between the plasmas prepared with different methods.

### Characterization of Hemophilia A Patients.

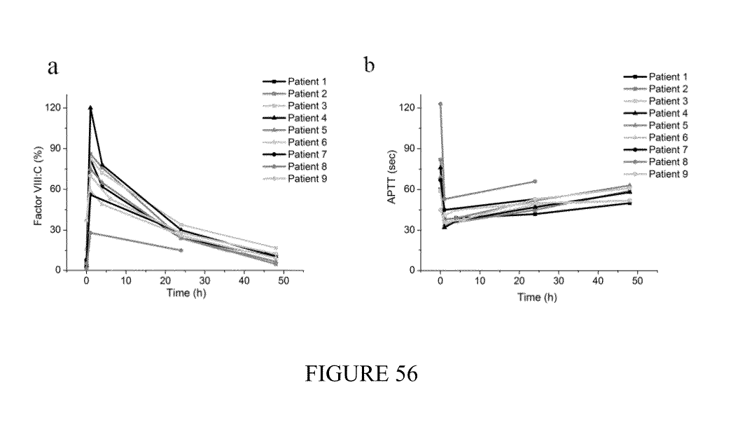

Pharmacokinetics experiments were performed using plasma from nine patients listed in Table 9. All patients were diagnosed with severe hemophilia A (FVIII:C<1%), were on regular prophylaxis except patient 8, and claimed to not have used Factor VIII concentrates for three days prior the experiment. Blood samples were collected before the administration of Factor VIII (timepoint 0 h), and at 1 and 24 hours after it. For all patients except for 7 and 8, additional samples were collected to determined Factor VIII clearance by determining Factor VIII level and APTT. They had normal pharmacokinetics (FIG. 56, Table 9) with the half-life of FVIII in the range of 5.5-20 h (Fischer et al, 2009; van Dijk et al, 2005) as determined using a single-exponent non-linear curve fit.

TABLE 9
Characterization of hemophilia A patients

| | | | | FVIII:C | | |
|---|---|---|---|---|---|---|
| | | | Monthly | on the | | Calculated |
| Patient | | | bleeding | day of the | APTT, | half-life |
| number | Age | Prophylaxis regiment | events | experiment, % | sec | for FVIII, h |
| 1 | 31 | Haemoctin, 50 IU/kg | 0 | 4.9 | 60 | 10.8 |
| 2 | 36 | Haemoctin, 50 IU/kg | 0 | 1.8 | 82 | 13.1 |
| 3 | 55 | Haemoctin, 50 IU/kg | 0 | 16 | 59 | 17.2 |
| 4 | 40 | Haemoctin, 50 IU/kg | 0 | 3.4 | 76 | 5.5 |
| 5 | 44 | Haemoctin, 50 IU/kg | 0 | 5.2 | 69 | 14.2 |
| 6 | 43 | Kogenate, 30 IU/ml | 1-2 | 7.5 | 60 | 19.6 |
| 7 | 25 | Octanate, 25 IU/kg | 0-1 | 7.6 | 76 | — |
| 8 | 25 | None* | 2-3 | <1 | 123 | — |
| 9 | 77 | Kogenate, 25 IU/kg | 1-2 | 37 | 45 | 16.1 |

*The patient was on an on-demand treatment. On the day of the experiment, he received Octanate at 19 IU/kg.

Factor VIII Interaction with BAX499 at Different Pharmacokinetics Timepoints

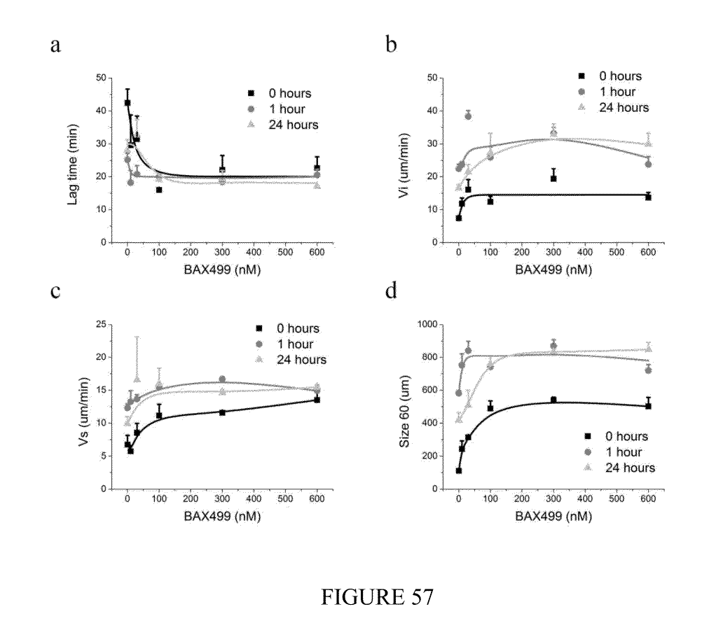

To study drug-drug interaction between BAX499 and Factor VIII, experiments using plasma from nine hemophilia A patients described above were performed. For each blood sample a number of spatial clot formation experiments in vitro with BAX499 concentration ranging from 0 to 600 nM were performed. The effects of BAX499 for the majority of patients were similar to that shown in FIG. 57. BAX499 improved coagulation at all timepoints reaching a plateau at ˜30-100 nM. Specifically, addition of BAX499 decreased lag time and increased clot growth velocity. This led to a significant increase in the integral parameter of total clot size. Importantly, the relative effect of BAX499 on clot size was higher in samples from patients at 0 and 24 h after FVIII administration, while it was smaller in 1 h samples, when the Factor VIII activity was maximal.

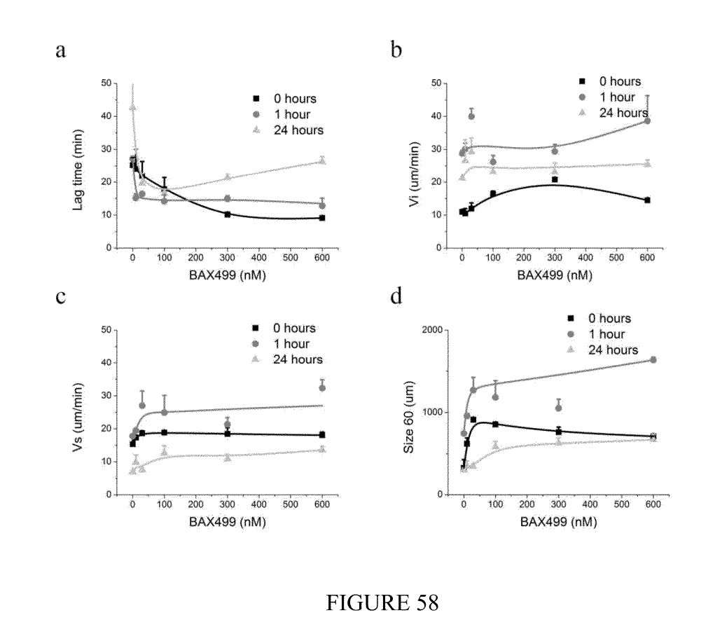

However, for two out of the nine patients the relative effect of BAX499 on clot size was similar for different time points of prophylaxis (FIG. 58). Clot size ratios for 600 nM BAX499 relative to baseline were ˜2.2 for all time points while FVIII:C activity was 1.8% at 0 h, 86% at 1 h, and 24% at 24 h.

BAX499 Efficiency as a Function of fVIII:C Activity

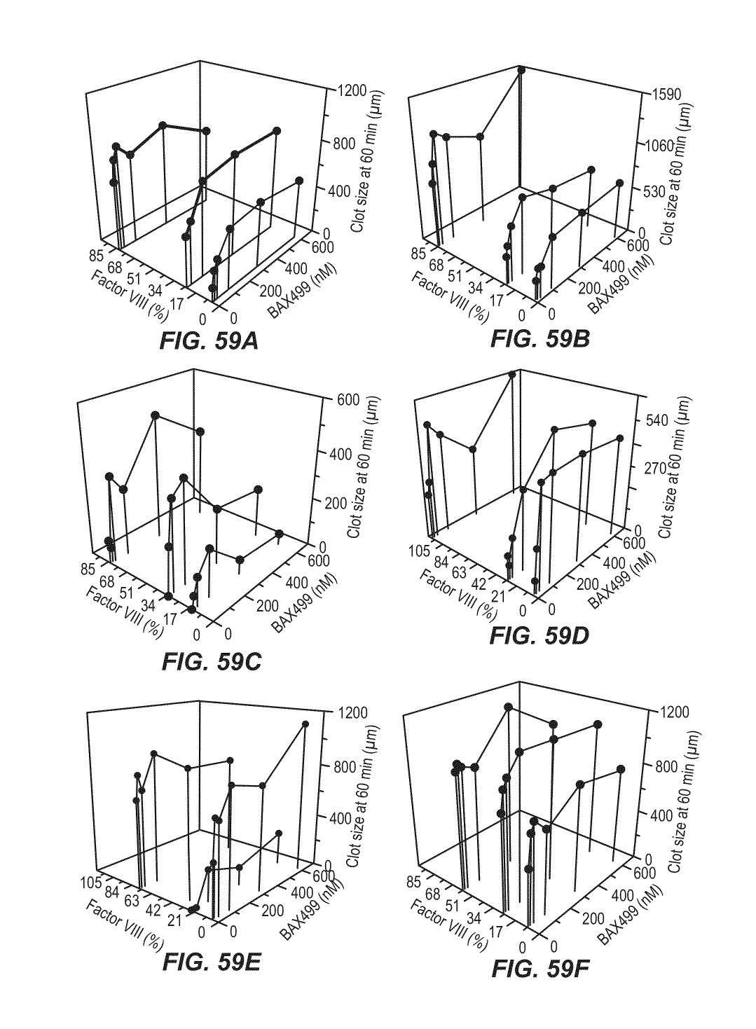

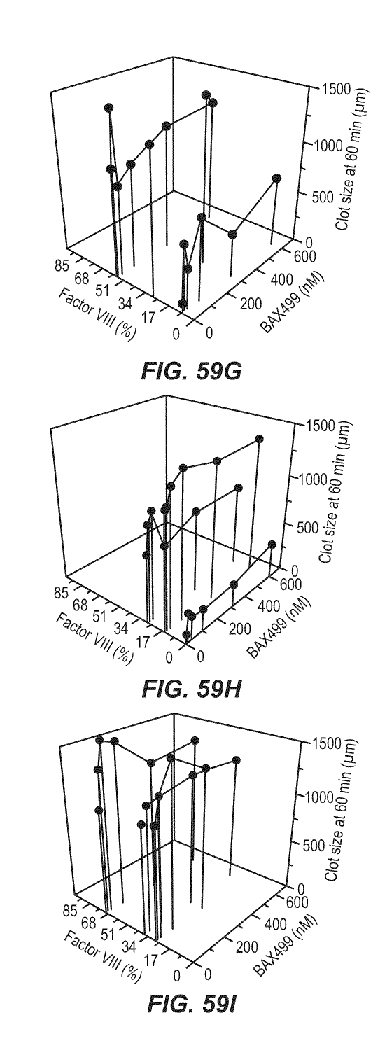

FIG. 59 illustrates combined effect of FVIII:C and BAX499 on clot size for all patients. In the majority of the studied patients (seven out of nine) the relative BAX499 effect on clot size was large at low Factor VIII activity and small at high Factor VIII activity. Additionally, the effect of Factor VIII was large at low BAX499 concentration. In the remaining two patients (panels b and c), the relative effect of BAX499 was similar for different Factor VIII levels.

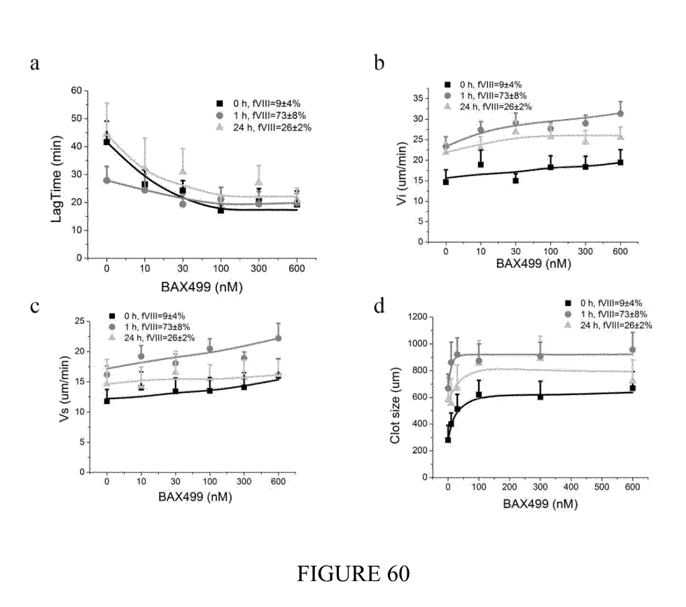

FIG. 60 illustrates statistical dose-dependence for BAX499 for each measured parameter at different time points in hemophilia A plasma. The data from the nine patients for the same time points and BAX499 concentrations was averaged. Lag time and clot size statistically increased until 100 nM of BAX499 for every time point with saturation at 30-100 nM. Initial and stationary velocity steadily increased within the whole range of BAX499.

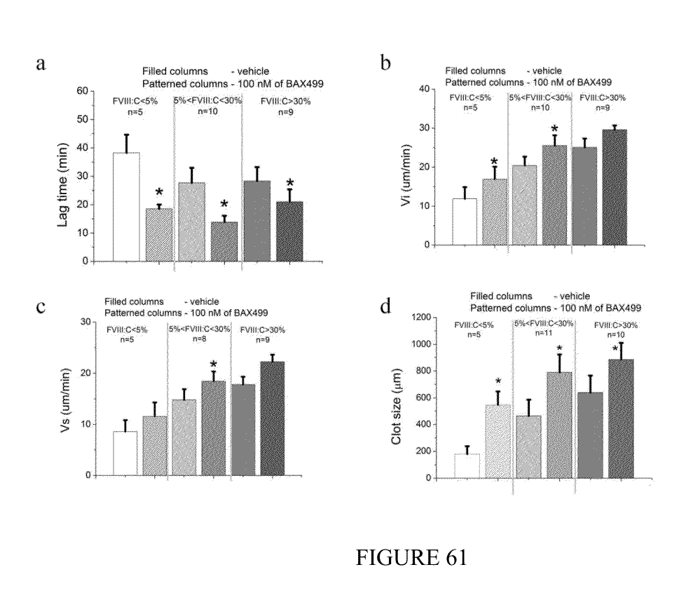

FIG. 61 shows averaged effects of saturating concentration of BAX499 on each measured parameter for three different ranges of Factor VIII concentration. In the presence of less than 30% of FVIII:C, BAX499 decreased the lag time 2.1-fold. In the range of 30-100% of Factor VIII activity, BAX499 addition decreased lag time only 1.3-fold. BAX499 increased spatial clot growth velocities only 1.2- to 1.4-fold. The most pronounced effect was observed for clot size. In the ranges of less than 5%, 5-30%, and more than 30% of Factor VIII activity, the clot size increased by 200%, 70% and 40% respectively. Therefore, as Factor VIII concentration increased, a BAX499 efficiency decrease was observed, although fibrin formation improved and remained significant for the whole range of concentrations.

### Possible Mechanism Behind the Factor VIII-BAX499 Interaction Effects

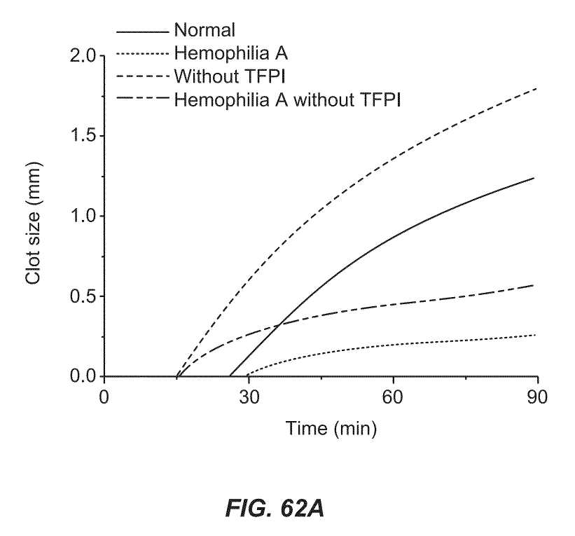

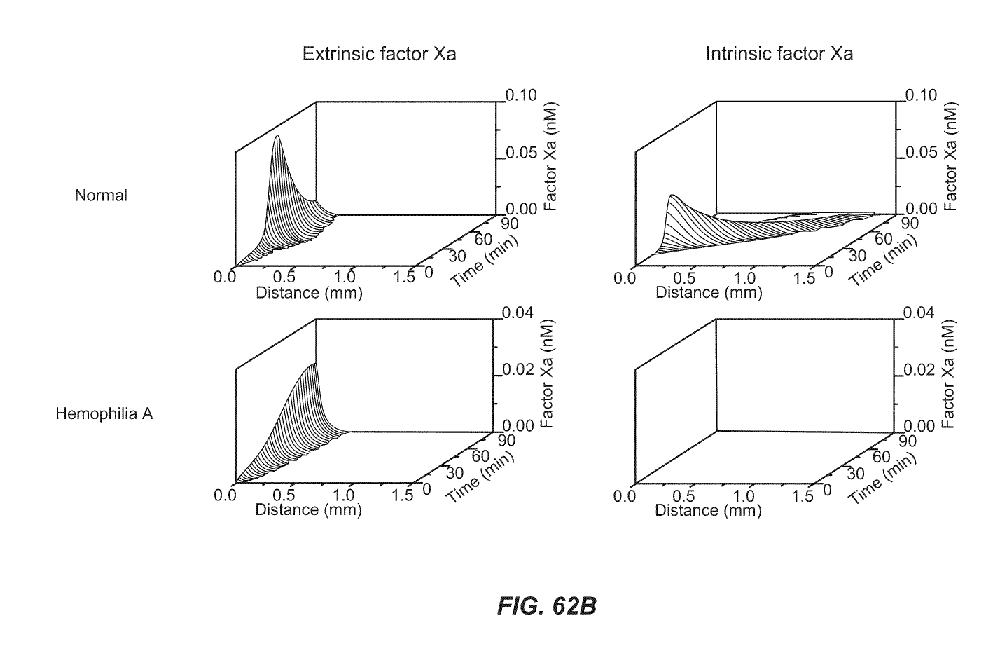

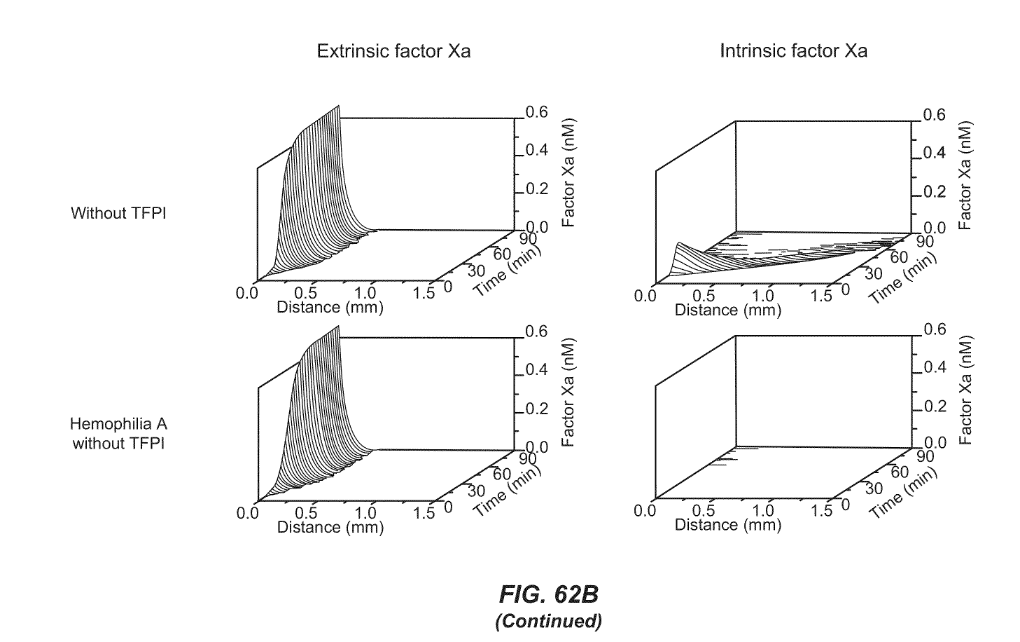

In order to gain insight into the mechanism of interaction of these two drugs, we performed computer simulation experiments of spatial fibrin clot formation using the mathematical model developed earlier. Factor VIII and BAX499 are known to promote coagulation via the two distinct pathways of Factor X activation. Factor VIII improves Factor Xa formation via acceleration of Factor IXa-dependent catalysis (i.e. intrinsic tenase) in its capacity of a co-factor; while BAX499 accelerated Factor X activation by the Factor VIIa-TF complex (i.e. extrinsic tenase) by preventing its inactivation by TFPI. To separate these contributions, concentration profiles were calculated of Factor Xa produced by extrinsic and intrinsic tenases in the mathematical model (FIG. 62). This was done for four cases: normal plasma (100% fVIII:C) with and without TFPI (2.5 nM or 0), and severe hemophilia A plasma (0% fVIII:C) with or without TFPI.

The mathematical model showed that the relative effect of TFPI removal should be smaller for high Factor VIII concentrations (FIG. 62A). The data on Factor Xa production by two different enzymes in the clotting system (FIG. 62B) showed that under normal conditions, Factor Xa produced by extrinsic tenase remains near the activator, while that produced by intrinsic tenase propagates into plasma and drives fibrin clot growth (FIG. 62B, row 1). In hemophilia, the propagation phase was impaired (FIG. 62B, row 2). Removal of TFPI greatly boosted near-activator Factor Xa production by extrinsic tenase by an order of magnitude (FIG. 62B, rows 3 and 4; note a different concentration scale), but this Factor Xa cannot rapidly get far from the activator because of its inhibition by plasma inhibitors (Panteleev et al, Biophys. J., 90, 1489-1500 (2006)).

The evaluation of the effect of the new anti-TFPI aptamer BAX499 on blood coagulation in the presence of different concentrations of Factor VIII in a spatial in vitro experimental model was determined. The main result of the study was that the relative effect of each agonist was usually decreased in the presence of high concentrations of another.

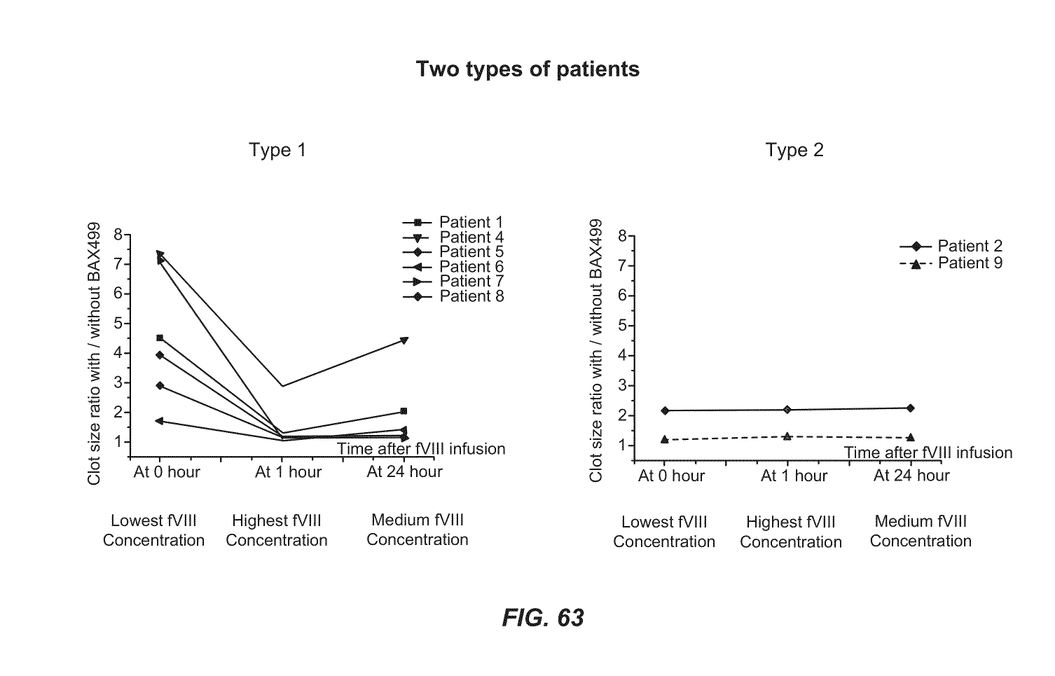

There were two exceptions to this: in two patients, the relative effects of BAX499 addition were similar for very different Factor VIII concentrations. In one of them (FIG. 59b), however, these effects were relatively small (˜100% increase in clot size in contrast to ˜300-400% effects observed in others at low Factor VIII concentrations). In another (FIG. 59c), the relative effects were more pronounced, which can possibly be explained by high TFPI concentration, but the absolute values of clot sizes were much smaller (less than 600 μm under all conditions, as compared with values of 1000-1500 μm observed in almost all others) suggesting that overdosing should not be expected to lead to thrombosis either. FIG. 63 illustrates the two types of patients.

BAX499 predominantly improved coagulation by acting on the initial stages of spatial fibrin clot formation, in contrast to Factor VIII that improves spatial propagation (Ovanesov et al, J. Thromb. Haemost., 3, 321-331 (2005); Ovanesov et al, Biochim. Biophys. Acta, 1572, 45-57 (2002); Panteleev et al, Biophys. J., 90, 1489-1500 (2006)). Computer simulations (FIG. 62) showed that this can be the major factor behind the phenomena of their drug-drug interaction. TFPI removal promoted Factor Xa production by extrinsic tenase by an order of magnitude, but this factor could not get far from the activator because of plasma inhibitors, and thus could not significantly influence spatial propagation. Because of their participation at two different, spatially and temporally distributed stages of coagulation process, the relative effect of one was smaller in the presence of another. The experiment supports this potential mechanism: clot size increase by saturating aptamer concentration was ˜400 μm for FVIII:C<5%, and only ˜200 μm for FVIII:C>30%.

Previous thrombin generation assay-based studies reported that activated prothrombin complex concentrates are likely to interact with Factor VIII synergistically (Livnat et al, Haemophilia., 14, 782-786 (2008); Klintman et al, Br. J. Haematol., 151, 381-386 (2010)), while activated Factor VII was found to work with Factor VIII in an additive fashion (Klintman et al, Br. J. Haematol., 151, 381-386 (2010)). The combination of VIIa and activated prothrombin complex concentrate was found to be either additive (Klintman et al, Br. J. Haematol., 151, 381-386 (2010)), or synergistic (Livnat et al, Haemophilia., 14, 782-786 (2008)), or one of the two depending on the patient (Martinowitz et al, Haemophilia., 15, 904-910 (2009)). BAX499 improves clotting at all Factor VIII concentrations, and vice versa so their combined addition can potentially somewhat improve the coagulation response. However, the mutual decrease of the effects appears to be a factor for the majority of the patients suggesting a potential for their use in combination, systematically or upon emergencies, without monitoring their levels.

### Example 13

### Drug-Drug Interactions Between BAX 499 and FYIII

Drug-drug interactions between BAX 499 and FVIII were tested in plasma from hemophilia A patients using the calibrated automated thrombogram (CAT) assay and in whole blood in a thromboelastometry assay using the ROTEM® system. The high concentrations used in these assays (2000 nM BAX 499 and 3 IU/mL FVIII) were chosen to reflect concentrations expected to be achieved after clinical administration of 1 mg/kg BAX 499 and 150 IU/kg FVIII, respectively. In both assays, the combination of the two compounds improved coagulation compared to each compound alone. In the CAT assay, it was evident that BAX 499 improved thrombin generation at concentrations from 10-50 nM compared to FVIII alone, but no significant additional benefit was seen when BAX 499 concentrations exceeded 50 nM. For both the CAT and ROTEM, the combination of 2000 nM BAX 499 and 3 IU/mL FVIII resulted in activity levels equivalent to 7.2 to 7.5 IU/mL FVIII.

### EXPERIMENTAL PROCEDURES

### Test Articles

BAX499 was diluted in 0.9% saline prior to use in the CAT assay, and in HNa-BSA5 buffer (25 mM HEPES, 175 mM NaCl₂, 5 mg/mL bovine serum albumin (BSA)) for use in the ROTEM assay. All concentrations of BAX499 are based on the oligonucleotide mass, excluding the polyethylene glycol moiety. Each vial of Advate® [recombinant antihemophilic Factor-protein free method; lot LEO1F523AB] was freshly reconstituted in sterile water immediately prior to use.

### Calibrated Automated Thrombogram® (CAT) Assay

Pooled plasma from patients with severe hemophilia A (<1% FVIII levels) was purchased from George King Bio-Medical (Overland Park, Kans., lot POOL-1928). Pooled normal plasma (PNP; George King Bio-Medical, cat no. 0010; lot 2084) was used as well. Thrombin calibrator (cat. no. TS20.00, lot TC1005/01), PPP-reagent LOW (cat. no. TS31.00, lot PPL0912/01), Fluo-Buffer and Fluo-Substrate (FluCa-kit, cat. no. TS50.00, lot FC1009/01) were all purchased from Diagnostica Stago (Parsippany, N.J.) and reconstituted as directed by the manufacturer. Assays were carried out in Immulon 2 HB—High Binding 96-well U-bottom plates (VWR, West Chester, Pa., cat. no. 62402-954).

### CAT Analysis of BAX 499 and FVIII

The drug-drug interaction between BAX 499 and FVIII was tested in hemophilia A plasma using the CAT assay. The plasma concentrations of BAX 499 were 1, 10, 50, 100, 500, 1000, and 2000 nM, and each concentration of BAX 499 was tested in the presence of 0, 0.01, 0.05, 0.3, 0.5, 1.0, 1.5, 2.25, and 3.0 IU/mL FVIII (Advate®). The chosen concentrations cover a wide range of BAX 499 and FVIII plasma concentrations up to values that are expected to be achieved after clinical administration of high doses of both test articles (up to 1 mg/kg BAX 499 and 150 IU/kg FVIII). FVIII was also tested in the absence of BAX 499 at the concentrations listed above, and at 5, 8.8 and 17.6 IU/mL in order to generate a standard curve for determination of FVIII equivalent activities (EA). Aptamer and/or FVIII were diluted in severe hemophilia A plasma to achieve the concentrations listed above. Plasma (80 μL) was then mixed with 20 μL PPP-reagent LOW and incubated for a few minutes at 37° C. in a 96-well U-bottom reaction plate. In control wells, 80 μL PNP or hemophilia plasma was mixed with 20 μL thrombin calibrator, and incubated at 37° C. The plate was loaded into a fluorescence plate-reader, and the reaction was started by the automated addition of 20 μL pre-warmed (37° C.) Flu-Ca reagent containing CaCl₂ and a fluorogenic substrate for thrombin. Tissue factor and phospholipids supplied by the PPP-reagent LOW were at 1 pM and 4 μM, respectively, in the final 120-4 mixture. Fluorescence intensity was measured every 20 seconds for 60 minutes.

### Instrumentation and Data Analysis

Thrombin generation via the CAT assay was measured using the Calibrated Automated Thrombogram® System, which consists of the Fluoroskan Ascent (Thermo Electron) fluorescence plate reader and Thrombinoscope analysis software, configured by Thrombinoscope BV (Maastricht, The Netherlands).

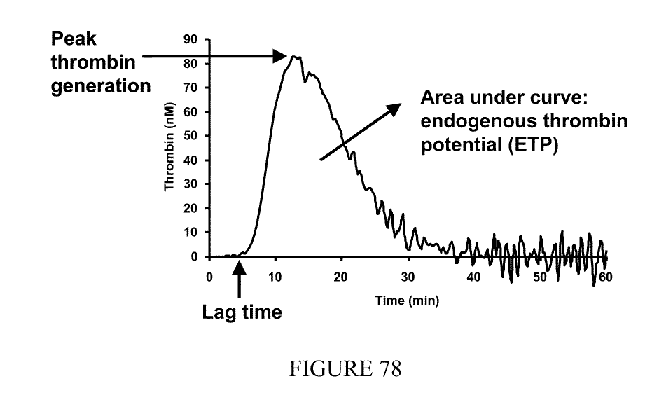

Analysis by Thrombinoscope BV software results in thrombin generation curves with thrombin (nM) on the y-axis and time (min) on the x-axis. The software also determines values for multiple parameters: endogenous thrombin potential (ETP; nM*min) is determined by the area under the thrombin generation curve; and peak thrombin (nM) is the highest amount of thrombin generated at any one point of the assay. A generic CAT tracing can be found in FIG. 78.

All experiments were performed five times. Results are reported as the mean±standard error (SEM) of the five replicates (n=5). The assay parameters were plotted individually against the concentration of BAX 499 or FVIII. A standard curve was generated by plotting the peak thrombin values at each concentration of FVIII alone and fitting to a 4-parameter logistics fit using GraphPad Prism 5 software. The equation from this fit was used to determine the FVIII EA for each BAX 499 condition (alone and in the presence of each FVIII concentration). The FVIII EA for each FVIII concentration in the absence of BAX 499 was also back-calculated using the equation from the standard curve fit to give a measure of the scatter associated with using the fit to determine FVIII EA.

### Whole Blood and ROTEM Reagents

Whole blood was freshly collected using a 20 G needle and 5-mL citrate S-Monovette tubes (3.2% tri-sodium citrate) containing corn trypsin inhibitor (61.75 μg/mL final concentration, Enzyme Research Labs, lot 336AL). The recalcification reagent CaCl₂ Star-TEM® (cat. no. 503-10, lot 41436401) and the ROTEM cuvettes were purchased from Ekomed. The tissue factor reagent PRP (cat. no. 42.00, lot 0910/01) was purchased from Thrombinoscope BV.

Continuous visco-elastic assessment of whole blood clotting was performed using a ROTEM coagulation analyzer at 37° C. BAX 499 was tested at 2 μM in normal whole blood (healthy single donor).

The FVIII concentration in the normal whole blood was assumed to be equivalent to 1 IU/mL. Drug-drug interaction of BAX 499 was studied in the presence of 1, 2 and 3 IU/mL FVIII (addition of 0, 1 and 2 IU/mL FVIII (Advate®) to normal whole blood, respectively). FVIII was also tested alone at added concentrations of 1 to 6 IU/mL to obtain a standard curve for determination of FVIII EA. Each sample was measured by pre-warming 20 μL of 0.2 M CaCl₂ and 20 μL of diluted tissue factor reagent in a ROTEM cuvette at 37° C. to give final concentrations of 11.76 mM and 44 fM, respectively. 4 μL of BAX 499 or HNa-BSA5 buffer was added to the pre-warmed blood 10 seconds prior to measurement to give a concentration of 2 μM in 300 μL blood volume. The ROTEM recording was started immediately following the addition of tissue factor and proceeded for at least 100 min.

### Instrumentation and Data Analysis

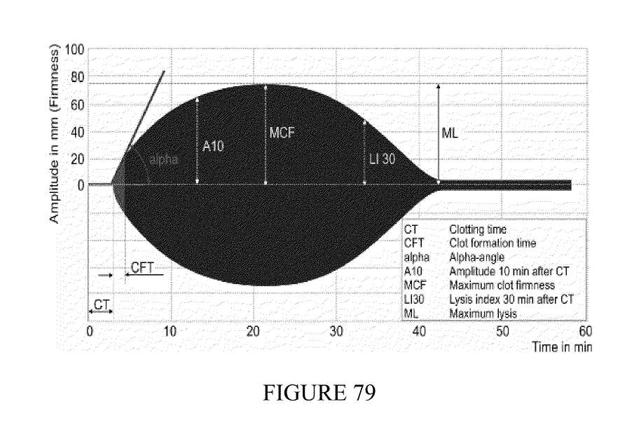

All measurements were performed in duplicate and are reported as the mean of the two measurements except for the FVIII EA of 2000 nM BAX 499 in the presence of 3 IU/mL FVIII EA, which is reported as the mean of the duplicate values determined from three independent experiments (CV=5%). Graphs show the mean±SEM. The ROTEM parameters such as clotting time (CT), clot formation time (CFT), alpha (α) and maximum clot firmness (MCF) were recorded in accordance with the manufacturer's instructions. A generic ROTEM tracing is illustrated in FIG. 79. The FVIII EA was determined for each BAX 499 condition. The FVIII standard curve was created by addition of FVIII alone to normal whole blood and analyzed using the 4-parameter logistics dynamic fit by Sigma Plot 11.0 software.

### BAX 499 and FVIII Activity in CAT Assay

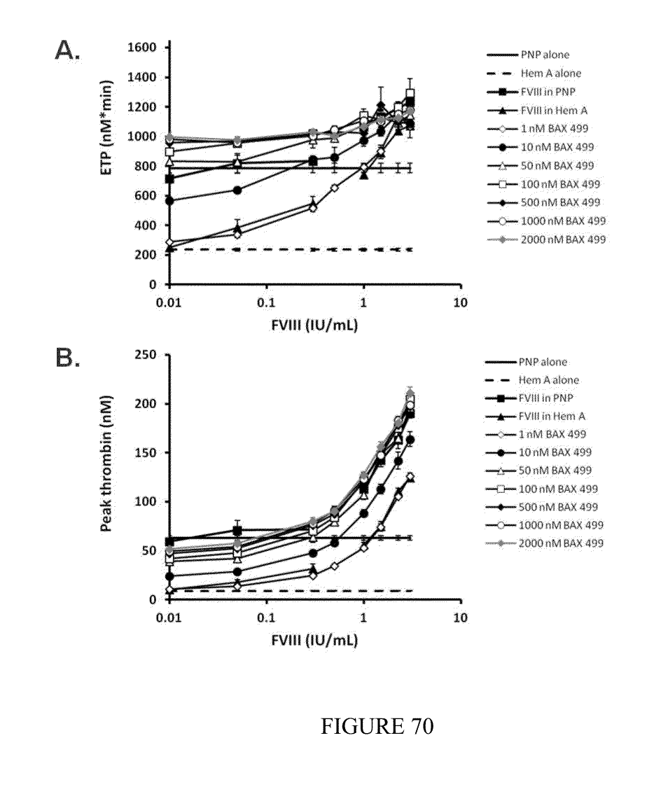

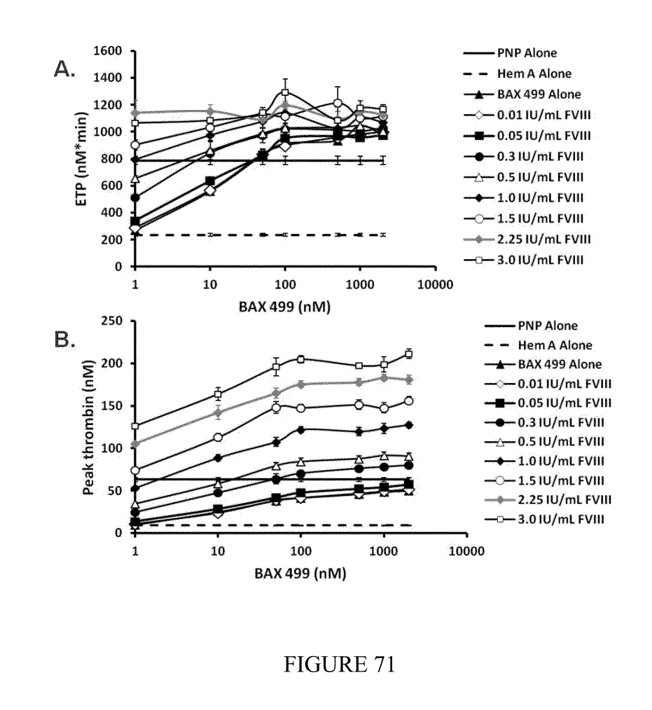

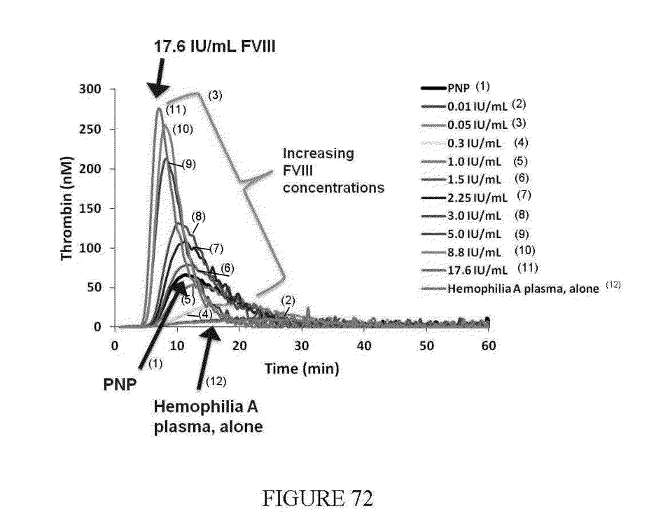

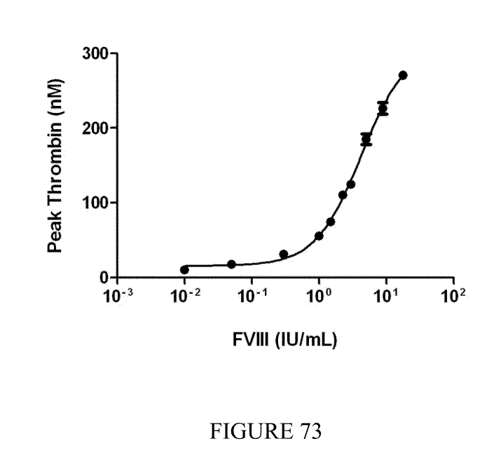

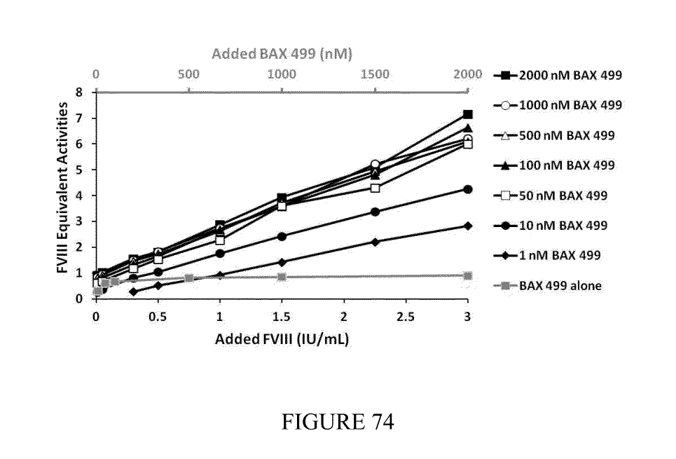

A dose response of BAX 499 in hemophilia A plasma was examined in the CAT assay in the presence of increasing concentrations of FVIII. All FVIII present in the CAT assays is recombinant FVIII (Advate®) and for simplicity will be referred to as “FVIII.” The ETP and peak thrombin values for these conditions are plotted as a function of FVIII concentration on the x-axis in FIG. 70A and FIG. 70B, respectively, and the same data are plotted as a function of BAX 499 concentration on the x-axis in FIG. 71A and FIG. 71B, respectively. A representative set of thrombin generation curves for FVIII concentrations in the absence of BAX 499 can be found in FIG. 72. A standard curve of peak thrombin values generated with FVIII alone is shown in FIG. 73. The FVIII EA of each BAX 499 concentration, either alone or in the presence of additional FVIII, was determined using this standard curve and can be found in FIG. 74 and Table 10.

TABLE 10
CAT: Conversion of BAX 499/ FVIII combinations to FVIII equivalent
activities (EA)

| | FVIII EA (IU/mL) | | | | | | | |
|---|---|---|---|---|---|---|---|---|
| FVIII | * 0 nM | 1 nM | 10 nM | 50 nM | 100 nM | 500 nM | 1000 nM | 2000 nM |
| (IU/mL) | BAX 499 | BAX 499 | BAX 499 | BAX 499 | BAX 499 | BAX 499 | BAX 499 | BAX 499 |
| 0 (ie, | - - -^{‡} | - - - | 0.3 | 0.6 | 0.7 | 0.8 | 0.8 | 0.9 |
| BAX 499 | | | | | | | | |
| alone) | | | | | | | | |
| 0.01 | - - - | - - - | 0.3 | 0.6 | 0.7 | 0.8 | 0.9 | 0.9 |
| 0.05 | 0.1 | - - - | 0.4 | 0.7 | 0.8 | 0.9 | 1.0 | 1.0 |
| 0.3 | 0.5 | 0.3 | 0.8 | 1.2 | 1.3 | 1.5 | 1.5 | 1.6 |
| 0.5 | n.d.† | 0.5 | 1.0 | 1.5 | 1.7 | 1.7 | 1.8 | 1.8 |
| 1 | 1.0 | 0.9 | 1.8 | 2.3 | 2.7 | 2.6 | 2.7 | 2.9 |
| 1.5 | 1.4 | 1.4 | 2.4 | 3.6 | 3.6 | 3.7 | 3.6 | 3.9 |
| 2.25 | 2.4 | 2.2 | 3.4 | 4.3 | 4.8 | 4.9 | 5.2 | 5.1 |
| 3 | 2.8 | 2.8 | 4.3 | 6.0 | 6.6 | 6.1 | 6.2 | 7.2 |

* The FVIII EA for each FVIII concentration was back-calculated using the equation from the standard curve fit to give a measure of the scatter associated with using the fit to determine FVIII EA;
^{‡}- - -: outside the range of the curve fit;
†n.d.: not determined

In general, ETP and peak thrombin values increased with increasing FVIII concentration (FIG. 70). Although 1 nM BAX 499 had no additional effect on ETP or peak thrombin compared to FVIII alone, BAX 499 concentrations ≧10 nM had an additive effect on FVIII, increasing the ETP and peak thrombin values compared to FVIII alone (FIG. 70). This additive effect plateaued at concentrations of 50 to 100 nM BAX 499, with higher concentrations of aptamer supplying no additional benefit. This was especially evident in FIG. 70B where the curves for BAX 499 concentrations ≧50 nM essentially overlap. The saturation of the BAX 499 effect is also illustrated in FIG. 71 where BAX 499 concentration is plotted on the x-axis and each data set represents a different FVIII concentration. ETP and peak thrombin values increased as the concentration of BAX 499 increased, up to approximately 100 nM BAX 499. At concentrations higher than 100 nM, a minimal increase in the ETP or peak thrombin values was observed. With the addition of ≧0.3 IU/mL FVIII, ETP and peak thrombin values increased compared to BAX 499 alone (FIG. 71).

Based on data shown in FIGS. 70 and 71, the maximal ETP achievable under these assay conditions appeared to be ˜1200 nM*min and may reflect an inherent property of hemophilia A plasma or the CAT assay. Due to this plateau at ˜1200 nM*min, ETP was not sufficiently sensitive to differentiate effects on thrombin generation at the higher concentrations. Peak thrombin values do not have the same limitations and, as such, are a more sensitive parameter in this assay. Therefore, peak thrombin values were determined from the thrombin generation curves of FVIII alone in hemophilia plasma (FIG. 72), and were used to generate a standard curve of FVIII activity for calculating FVIII EA (FIG. 73). Low concentrations of FVIII had a minimal effect on peak thrombin values, evidenced by the fact that the standard curve is nearly flat at concentrations below 0.3 IU/mL. As such, it was expected that EA values calculated from peak thrombin values in the lower range of the curve would have more variance.

Using the standard curve (FIG. 73), FVIII EA was determined for each concentration of BAX 499, either alone or in combination with FVIII. The FVIII EAs achieved with BAX 499 alone were 0.3 to 0.9 IU/mL over the concentration range of 10 to 2000 nM BAX 499 (FIG. 74; Table 10); 1 nM BAX 499 had no impact on the FVIII EA. The addition of 0.01 IU/mL FVIII showed no further increase in FVIII EA when compared to BAX 499 alone across the concentration range of BAX 499 tested (Table 10). When tested in combination with FVIII, 1 nM BAX 499 had no additional impact on EA compared to FVIII alone (Table 10). At each FVIII concentration above 0.01 IU/mL, the addition of both 10 and 50 nM BAX 499 increased the EA by approximately 1.5- to 2.5-fold when compared to FVIII alone. Minimal additional change in FVIII EAs (1.0- to 1.3-fold) was observed at BAX 499 concentrations greater than 50 nM at each FVIII concentration (Table 10). This is also shown in FIG. 74 where it can be seen that the lines associated with BAX 499 concentrations ≧50 nM are nearly identical. Additionally, at each BAX 499 concentration, the FVIII EAs increased linearly as a function of FVIII concentration for FVIII concentrations ≧0.05 IU/mL (FIG. 74). The maximum FVIII EA observed in these experiments was 7.2 IU/mL, which occurred when 2000 nM BAX 499 was tested in combination with 3 IU/mL FVIII.

### BAX 499 and FVIII in ROTEM Assay

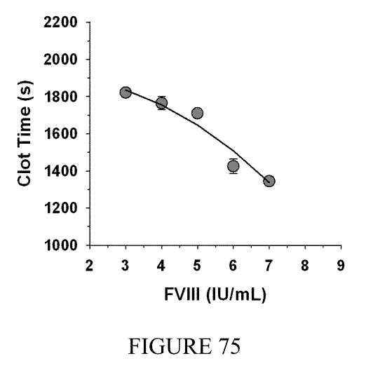

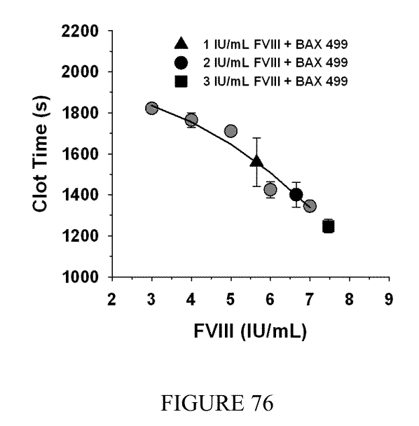

ROTEM investigations of BAX 499 were performed in the presence of increasing concentrations of FVIII in normal whole blood. Because this assay was performed in normal whole blood, the first IU/mL of FVIII is endogenous and additional units are the result of exogenously-added recombinant FVIII (Advate®). FVIII was tested alone to generate a standard curve. Clot time (CT) values for these conditions are plotted as a function of FVIII concentration (IU/mL) in FIG. 75 and assume that normal whole blood contains the equivalent of 1 IU/mL FVIII. ROTEM investigations were also carried out in the presence of both FVIII and BAX 499. For these experiments, only the highest concentration of BAX 499 tested in the CAT assay (2000 nM) was tested in combination with different FVIII concentrations because the ROTEM assay is much lower throughput than the CAT assay. This concentration of BAX 499 should provide an upper limit of the impact of the BAX 499 and FVIII combinations in these assays under the tested conditions. The FVIII EA of 2000 nM BAX 499 in the presence of 1, 2 and 3 IU/mL FVIII was determined from the standard curve shown in FIG. 75. These data are plotted along with the standard curve in FIG. 76 and the values are shown in Table 11.

TABLE 11
ROTEM: Conversion of BAX 499/ FVIII combinations to FVIII
equivalent activities (EA); BAX 499 was only tested at 2000 nM

| | FVIII | | | | | |
|---|---|---|---|---|---|---|
| | added | | | | | |
| | to | | FVIII + | **FVIII | FVIII | Fold |
| | whole | FVIII | 2 μM | EA | + 2 μM | increase |
| *FVII | blood | alone | BAX 499 | alone | BAX 499 | over FVIII |
| (IU/mL) | (IU/ml) | (CT s) | (CT s) | (IU/mL) | (IU/mL) | alone |
| 1 | — | — | 1559 | — | 5.7 | 5.7 |
| 2 | 1 | — | 1401 | — | 6.7 | 3.3 |
| 3 | 2 | 1822 | 1247 | 3.1 | 7.5^{‡} | 2.5 |
| 4 | 3 | 1740 | — | 4.2 | — | — |
| 5 | 4 | 1710 | — | 4.5 | — | — |
| 6 | 5 | 1464 | — | 6.3 | — | — |
| 7 | 6 | 1345 | — | 6.9 | — | — |

*Values assume that the FVIII in normal whole blood is equivalent to 1 IU/mL.
**Values in this column where determined by fitting the FVIII concentrations used in the standard curve (R = 0.957) to the same quadratic polynomial equation logistics as the rest of the data;
^{‡}outside the range of the curve fit

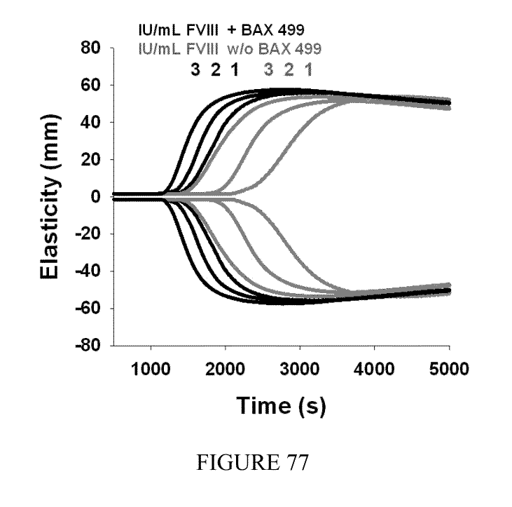

ROTEM experiments were performed with normal whole blood to examine the effect of combinations of BAX 499 and FVIII on coagulation parameters. The addition of BAX 499 to blood containing different concentrations of FVIII improved coagulation parameters as compared to FVIII alone, as shown in FIG. 77. This graph shows the amplitude of the ROTEM traces of different FVIII concentrations with and without 2000 nM BAX 499 in whole blood. A decrease in the clot time on the x-axis was observed in the samples containing BAX 499 (black) as compared to the samples without BAX 499 (gray). The level of coagulation achieved with each BAX 499/FVIII combination was not limited by the assay itself (data not shown).

Table 11 shows the magnitude of the BAX 499 effect (“fold increase”) by ROTEM, which decreased as the concentration of FVIII increased, but was still ˜2.5-fold at the highest concentration of FVIII (3 IU/mL). Similar results were observed in the CAT assay for the FVIII EA of 2000 nM BAX 499 with 3 IU/mL FVIII.

At the highest tested concentrations of both BAX 499 (2000 nM) and FVIII (3 IU/mL), the resulting response in the ROTEM assay was equivalent to 7.5 IU/mL of FVIII (Table 10), which was similar to the value of 7.2 IU/mL obtained in the CAT assay with the same BAX 499 and FVIII concentrations (Table 11).

Combining BAX 499 with FVIII on coagulation in vitro using both the CAT and the ROTEM assays is shown in this example. In the CAT assay, BAX 499 alone reached an activity level equivalent to 0.9 IU/mL FVIII at the highest concentration tested (2000 nM). The combined effect of the two molecules differed at the different concentration levels. At low concentrations of either BAX 499 or FVIII, neither compound affected the activity of the other (i.e, 1 nM BAX 499 had no additive effect on thrombin generation compared to FVIII alone, and 0.01 IU/mL FVIII had no additional effect compared to BAX 499 alone). At the mid-concentration range (10-50 nM BAX 499), activity increased approximately 1.5-2.5-fold over FVIII alone, suggesting that coagulation was improved with the combination of both drugs. The addition of BAX 499 at concentrations >50 nM did not appear to have a significant added benefit. At BAX 499 concentrations of 100 to 2000 nM, the amount of thrombin generation was explicitly dependent on FVIII concentration. In the ROTEM assay, only one concentration of BAX 499 (2000 nM) and high concentrations of FVIII (1-3 IU/mL) were tested. Similar to what was observed in the CAT assay, BAX 499 improved the clot time compared to FVIII alone. In both assays, the combination of 2000 nM BAX 499 and 3 IU/mL FVIII resulted in activity levels equivalent to 7.2 IU/mL (CAT) and 7.5 IU/mL (ROTEM) FVIII.

### Example 14

### Performance of Stored BAX 499

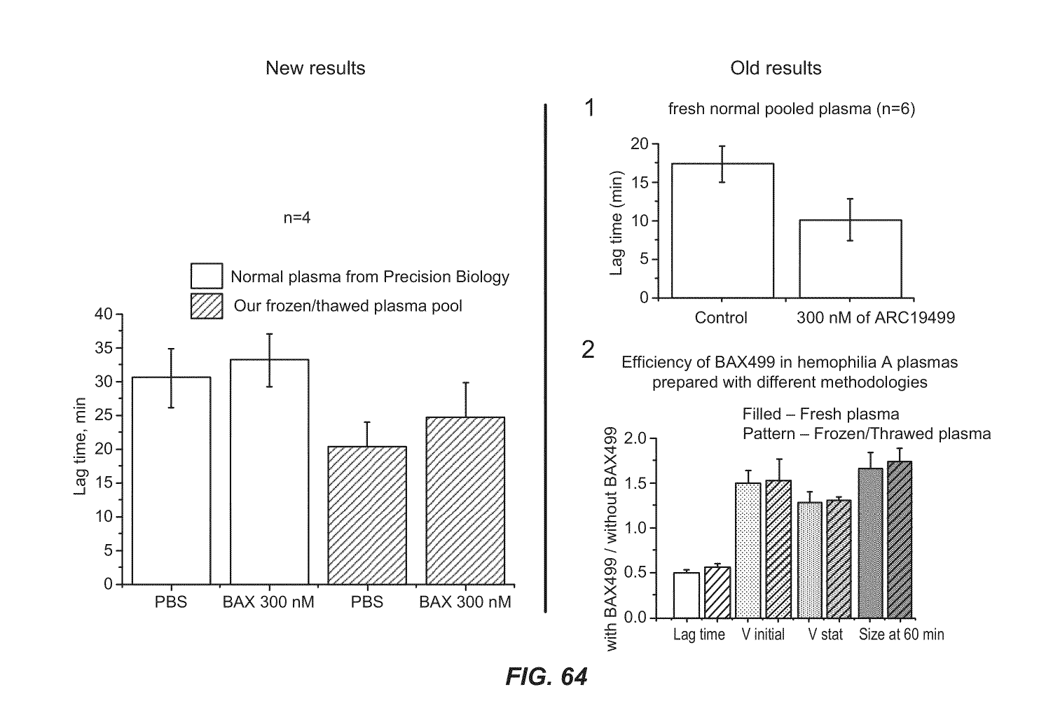

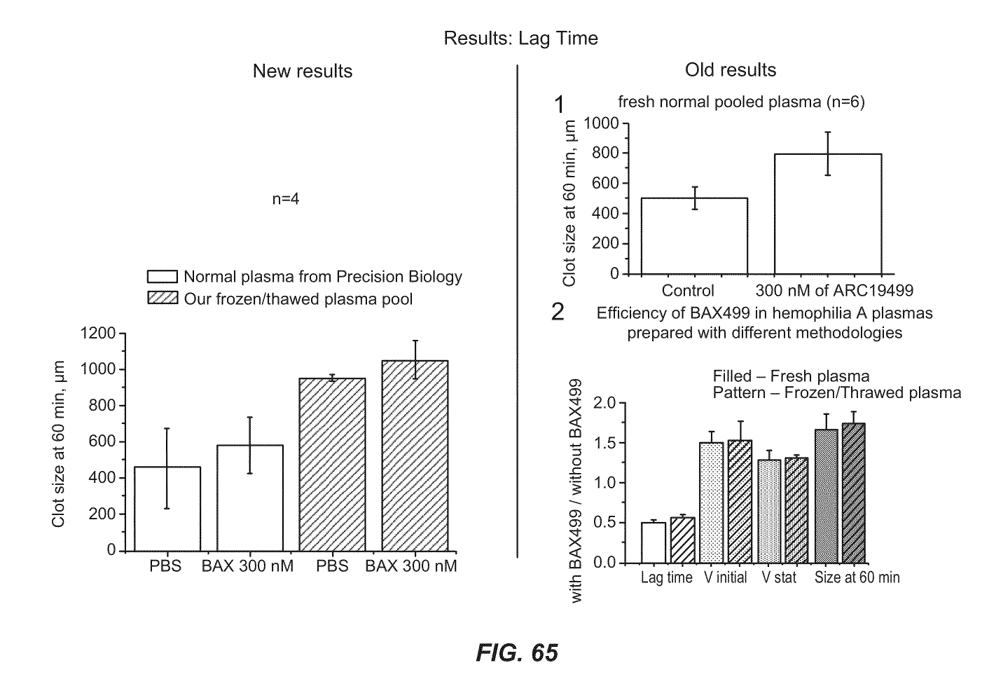

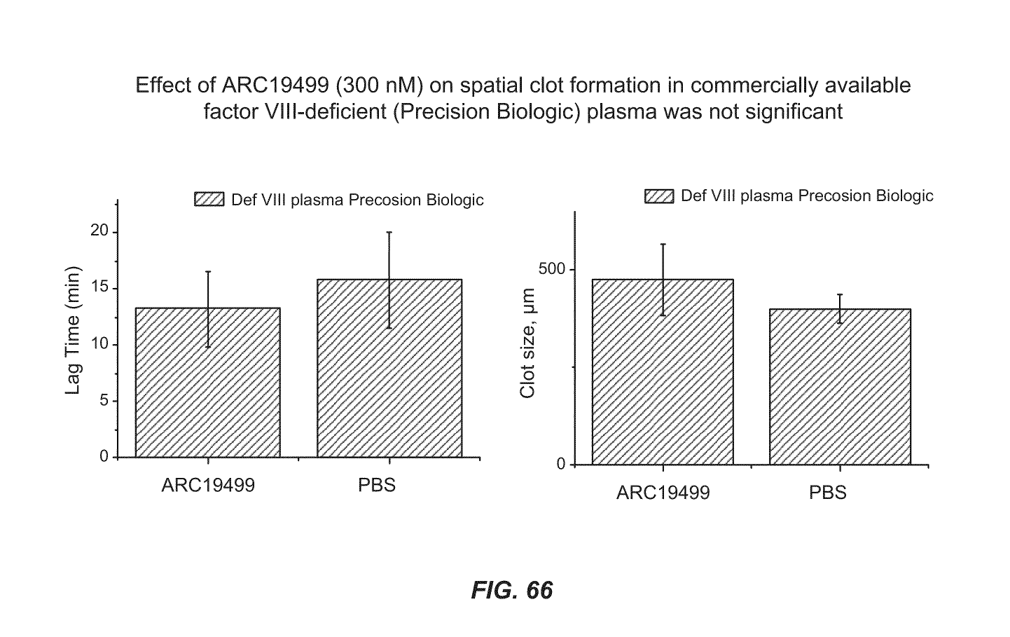

A new solution of BAX 499 was prepared from dry BAX 499 that was stored for 2 years at +4° C. The solution was aliquoted and frozen at −80° C. The experiments were performed with frozen plasma (Precision Biologic) and in house frozen/thawed pooled plasma. Control experiments with addition of PBD (n=4) and with 300 nM of BAX 499 (n=4) were performed (FIGS. 64-66).

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual and Molecular Cloning: A Laboratory Manual (all from Cold Spring Harbor Laboratory Press), Stryer, L. (1995) Biochemistry (4th Ed.) Freeman, Highly stabilized York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., Highly stabilized York, N.Y. and Berg et al. (2002) Biochemistry, 5^{th} Ed., W.H. Freeman Pub., Highly stabilized York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the above description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

1. A method of increasing plasma TFPI protein levels in a subject, said method comprising administering BAX499 to said subject.

2. The method of claim 1, wherein said subject has a clotting disorder.

3. The method of claim 1, wherein BAX499 modulates binding of TFPI to LRP-1.

4. The method of claim 1, wherein BAX499 reduces TFPI clearance, thereby increasing TFPI half-life.

5. A method of increasing procoagulant activity in a subject, said method comprising administering BAX499 to said subject at a dose effective for increasing said procoagulant activity.

6. The method of claim 5, wherein said dose is in a range of from about 3 mg to about 72 mg.

7. The method of claim 5, wherein said dose is in a range of from about 5 nM to about 1000 nM.

8. A method of determining a pharmaceutically effective dose of BAX499, said method comprising assaying blood clotting in samples obtained from subjects administered with differing doses of BAX499.

9. The method of claim 8, wherein said subjects are hemophilia patients.

10. The method of claim 8, wherein said subjects have been infused with FVIII prior to, simultaneously with, or subsequent to administration with BAX499.

11. The method of claim 8, wherein said assaying comprises a member selected from: assaying clotting time, assaying clot stability, and assaying clot size.

12. A method of mobilizing intracellular TFPI protein in a subject, said method comprising administering a pharmaceutically effective dose of BAX499.

13. The method of claim 12, wherein said dose is in a range of from about 3 mg to about 72 m.

14. The method of claim 12, wherein said dose is in a range of from about 5 nM to about 1000 nM.

15. The method of claims 12, wherein said pharmaceutically effective dose of BAX-199 does not increase TFPI mRNA levels in said subject.

16. A method of increasing the procoagulant effect of FVIII in a subject, said method comprising co-administering BAX499 and FVIII to said subject.

17. The method of claim 16, wherein said co-administered FVIII is recombinant FVIII.

18. The method of claims 16, wherein said increase in the procoagulant effect is about 1.5 to about 7-fold higher than the increase seen with FVIII alone.

19. A method of increasing anticoagulant activity in a subject, said method comprising administering BAX499 to said subject at a dose effective for increasing said anticoagulant activity.

20. A method of determining whether a molecule inhibits or activates TFPI protein function, increases or decreases intracellular or membrane bound TFPI plasma protein levels, or increases or decreases TFPI clearance, said method comprising conducting a competitive assay with said molecule and BAX499.

21. The method of claim 20, wherein said competitive assay is a competitive binding assay.

22. The method of claim 20, wherein said molecule is a member selected from an aptamer, a peptide, an antibody, and a small molecule.

23. The method of claim 20, wherein the determining step measures TFPI protein function.

24. The method of claim 20, wherein the determining step measures intracellular or membrane bound TFPI plasma concentration.

25. The method of claim 20, wherein the determining step measures TFPI clearance.

26. The method of claim 20, wherein the molecule inhibits TFPI protein function.

27. The method of claim 20, wherein the molecule activates TFPI protein function.

28. The method of claim 20, wherein the molecule increases intracellular or membrane bound TFPI plasma protein levels.

29. The method of claim 20, wherein the molecule decreases intracellular or membrane bound TFPI plasma protein levels.

30. The method of claim 20, wherein the molecule increases TFPI clearance.

31. The method of claim 20, wherein the molecule decreases TFPI clearance.
